(19) United States
(12) Patent Application Publication
(10) Pub. No.: US 2019/0055510 A1
(43) Pub. Date: Feb. 21, 2019

(54) WELL PLATE-BASED PERFUSION CULTURE MODEL OF ENDOSTEAL-EXTRACELLULAR MATRIX (ECM)-AND ENDOTHELIAL-MYELOMA INTERACTIONS AND METHODS FOR TESTING PERSONALIZED THERAPEUTICS FOR MULTIPLE MYELOMA

(71) Applicants: Hackensack University Medical Center, Hackensack, NJ (US); Stevens Institute of Technology, Hoboken, NJ (US)

(72) Inventors: Woo Young Lee, Lyndhurst, NJ (US); Wenting Zhang, Kearny, NJ (US); Yexin Gu, Shanghai (CN); Yi Hao, New York, NY (US); Qiaoling Sun, Secaucus, NJ (US); Jenny Zilberberg, Yardley, PA (US)

(21) Appl. No.: 15/719,737

(22) Filed: Sep. 29, 2017

Related U.S. Application Data
(60) Provisional application No. 62/401,661, filed on Sep. 29, 2016.

(51) Int. Cl. C12M 1/34; C12M 1/32; C12M 1/00; G01N 33/50

(52) U.S. Cl. CPC C12M 41/46; C12M 23/12; C12M 29/10; G01N 33/5011; C12M 23/22; G01N 33/5088; C12M 23/38

(57) ABSTRACT

The described invention provides a well plate-based perfusion culture model of endosteal-, extracellular matrix (ECM)- and endothelial-myeloma interactions and patient-specific methods for selecting treatment for and assessing drug resistance of multiple myeloma (MM). The described methods utilize an ex vivo three dimensional endosteal microenvironment effective to recapitulate spatial and temporal characteristics of a multiple myeloma cancer niche and to maintain viability of multiple myeloma cells (MMCs) obtained from a patient suffering from MM.

### CROSS-REFERENCE TO RELATED APPLICATIONS

This application claims the benefit of priority to U.S. provisional patent application Ser. No. 62/401,661, filed Sep. 29, 2016, which claims the benefit of priority to PCT/US2016/012573, filed Jan. 8, 2016, which claims priority to U.S. provisional patent application Ser. No. 62/101,181, filed Jan. 8, 2015, the content of each of these applications is incorporated by reference herein in their entirety.

### FIELD OF INVENTION

The described invention relates to an ex-vivo well plate-based perfusion culture model of cell to cell interactions, and methods for testing personalized therapeutics using the model.

### BACKGROUND OF THE INVENTION

### Tissue Compartments, Generally

In multicellular organisms, cells that are specialized to perform common functions are usually organized into cooperative assemblies embedded in a complex network of secreted extracellular macromolecules, the extracellular matrix (ECM), to form specialized tissue compartments. Individual cells in such tissue compartments are in contact with ECM macromolecules. The ECM helps hold the cells and compartments together and provides an organized lattice or construct within which cells can migrate and interact with one another. In many cases, cells in a compartment can be held in place by direct cell-cell adhesion. In vertebrates, such compartments may be of four major types, a connective tissue (CT) compartment, an epithelial tissue (ET) compartment, a muscle tissue (MT) compartment and a nervous tissue (NT) compartment, which are derived from three embryonic germ layers: ectoderm, mesoderm and endoderm. The NT and portions of the ET compartments are differentiated from the ectoderm; the CT, MT and certain portions of the ET compartments are derived from the mesoderm; and further portions of the ET compartment are derived from the endoderm.

### The Bone Marrow Niche

The term “niche” as used herein refers to a specialized regulatory microenvironment, consisting of components which control the fate specification of stem and progenitor cells, as well as maintaining their development by supplying the requisite factors. The term “bone marrow (BM) niche” as used herein refers to a well-organized architecture composed of osteoblasts, osteoclasts, bone marrow endothelial cells, stromal cells, adipocytes and extracellular matrix proteins (ECM). These elements play an essential role in the survival, growth and differentiation of diverse lineages of blood cells.

Bone marrow consists of a variety of precursor and mature cell types, including hematopoietic cells (the precursors of mature blood cells) and stromal cells (the precursors of a broad spectrum of connective tissue cells), both of which appear to be capable of differentiating into other cell types. The mononuclear fraction of bone marrow contains stromal cells, hematopoietic precursors, and endothelial precursors.

### Extracellular Matrix (ECM) Proteins

The ECM is a complex structural entity surrounding and supporting cells found within mammalian tissues. The ECM is comprised of proteoglycans (e.g., heparan sulfate, chondroitin sulfate, keratin sulfate, hyaluronic acid), collagen, fibronectin, laminin and elastin. Most mammalian cells cannot survive unless they are anchored to the ECM. Cells attach to the ECM via transmembrane glycoproteins (e.g., integrins) which bind to various types of ECM proteins (e.g., collagens, laminins, fibronectin).

### Adipocytes

Adipocytes of the bone marrow stroma provide the cytokines and extracellular matrix proteins required for the maturation and proliferation of the circulating blood cells. Due to the complexity of the bone marrow as an organ, the normal physiology of these stromal cells is not well understood. In particular, the role of adipocytes in the bone marrow remains controversial. Cloned bone marrow stromal cell lines provide an in vitro model for analysis of the lympho-hematopoietic microenvironment. These cells may be capable of multiple differentiation pathways, assuming the phenotype of adipocytes, chondrocytes, myocytes, and osteocytes in vitro. (Gimble J M, New Biol., 1990 April; 2(4): 304-312).

### Hematopoietic Stem Cells Development and Maintenance

Hematopoietic stem cells (HSCs) (also known as the colony-forming unit of the myeloid and lymphoid cells (CFU-M,L), or CD34+ cells) are rare pluripotential cells within the blood-forming organs that are responsible for the continued production of blood cells during life. While there is no single cell surface marker exclusively expressed by hematopoietic stem cells, it generally has been accepted that human HSCs have the following antigenic profile: CD 34+, CD59+, Thy1+(CD90), CD38low/−, C-kit−/low and, lin− (Chotinantakul, K. and Leeanansaksiri, W., Bone Marrow Research, Vol. 2012, Article ID 270425; The National Institutes of Health, Resource for Stem Cell Research, http://stemcells.nih.gov/info/scireport/pages/chapter5.aspx). CD45 is also a common marker of HSCs, except platelets and red blood cells (The National Institutes of Health, Resource for Stem Cell Research, http://stemcells.nih.gov/info/scireport/pages/chapter5.aspx). HSCs can generate a variety of cell types, including erythrocytes, neutrophils, basophils, eosinophils, platelets, mast cells, monocytes, tissue macrophages, osteoclasts, and the T and B lymphocytes (The National Institutes of Health, Resource for Stem Cell Research, http://stemcells.nih.gov/info/scireport/pages/chapter5.aspx). The regulation of hematopoietic stem cells is a complex process involving self-renewal, survival and proliferation, lineage commitment and differentiation and is coordinated by diverse mechanisms including intrinsic cellular programming and external stimuli, such as adhesive interactions with the micro-environmental stroma and the actions of cytokines (Chotinantakul, K. and Leeanansaksiri, W., Bone Marrow Research, Vol. 2012, Article ID 270425; The National Institutes of Health, Resource for Stem Cell Research, http://stemcells.nih.gov/info/scireport/pages/chapter5.aspx).

Different paracrine factors are important in causing hematopoietic stem cells to differentiate along particular pathways. Paracrine factors involved in blood cell and lymphocyte formation are called cytokines. Cytokines can be made by several cell types, but they are collected and concentrated by the extracellular matrix of the stromal (mesenchymal) cells at the sites of hematopoiesis (Majumdar, M. K. et al., J. Hematother. Stem Cell Res. 2000 December; 9(6): 841-848). For example, granulocyte-macrophage colony-stimulating factor (GM-CSF) and the multilineage growth factor IL-3 both bind to the heparan sulfate glycosaminoglycan of the bone marrow stroma (Burdon, T. J., et al., Bone Marrow Research, Volume 2011, Article ID 207326; Baraniak, P. R. and McDevitt, T. C., Regen. Med. 2010 January; 5(1): 121-143). The extracellular matrix then presents these factors to the stem cells in concentrations high enough to bind to their receptors.

### Mesenchymal Stem Cells (MSCs)

Mesenchymal stem cells (MSCs) (also known as bone marrow stromal stem cells or skeletal stem cells) are non-blood adult stem cells found in a variety of tissues. They are characterized by their spindle-shape morphologically; by the expression of specific markers on their cell surface; and by their ability, under appropriate conditions, to differentiates along a minimum of three lineages (osteogenic, chondrogenic, and adipogenic) (Minguell, J. J., et al., Experimental Biology and Medicine 2001, 226: 507-520; Tuan, R. S., et al., Arthritis Res. Ther. DOI: 10.1186/ar614).

No single marker that definitely delineates MSCs in vivo has been identified due to the lack of consensus regarding the MSC phenotype, but it generally is considered that MSCs are positive for cell surface markers CD105, CD166, CD90, and CD44 and that MSCs are negative for typical hematopoietic antigens, such as CD45, CD34, and CD14 (Minguell, J. J., et al., Experimental Biology and Medicine 2001, 226: 507-520; Lee, H. J., et al., Arthritis & Rheumatism, Vol. 60, No. 8, August 2009, pp. 2325-2332; Kolf, C. M., et al., Arthritis Research & Therapy 2007, 9:204, DOI: 10.1186/ar2116). As for the differentiation potential of MSCs, studies have reported that populations of bone marrow-derived MSCs have the capacity to develop into terminally differentiated mesenchymal phenotypes both in vitro and in vivo, including bone, cartilage, tendon, muscle, adipose tissue, and hematopoietic-supporting stroma (Gimbel, J. M., et al., Transfus. Med. Hemother. 2008; 35: 228-238; Minguell, J. J., et al., Experimental Biology and Medicine 2001, 226: 507-520; Kolf, C. M., et al., Arthritis Research & Therapy 2007, 9:204, DOI: 10.1186/ar2116). Studies using transgenic and knockout mice and human musculoskeletal disorders have reported that MSC differentiate into multiple lineages during embryonic development and adult homeostasis (Komine, A., et al., Biochem. Biophys. Res. Commun. 2012 Oct. 5; 426(4): 468-474; Shen, J., et al., Scientific Reports, 1:67, DOI: 10.1038/srep00067; Reiser, J., et al., Expert. Opin. Biol. Ther. 2005 December; 5(12): 1571-1584).

Analyses of the in vitro differentiation of MSCs under appropriate conditions that recapitulate the in vivo process have led to the identification of various factors essential for stem cell commitment. Among them, secreted molecules and their receptors (e.g., transforming growth factor-β), extracellular matrix molecules (e.g., collagens and proteoglycans), the actin cytoskeleton, and intracellular transcription factors (e.g., Cbfal/Runx2, PPAR, Sox9, and MEF2) have been shown to play important roles in driving the commitment of multipotent MSCs into specific lineages, and maintaining their differentiated phenotypes (Kolf, C. M., et al., Arthritis Research & Therapy 2007, 9:204, DOI: 10.1186/ar2116).

For example, it has been shown that osteogenesis of MSCs, both in vitro and in vivo, involves multiple steps and the expression of various regulatory factors. During osteogenesis, multipotent MSCs undergo asymmetric division and generate osteoprecursors, which then progress to form osteoprogenitors, preosteoblasts, functional osteoblasts, and eventually osteocytes (Bennett, K. P., et al., BMC Genomics 2007, 8:380, DOI: 10.1186/1471-2164-8-380). This progression from one differentiation stage to the next is accompanied by the activation and subsequent inactivation of transcription factors, i.e., Cbfal/Runx2, Msx2, Dlx5, Osx, and expression of bone-related marker genes, i.e., osteopontin, collagen type I, alkaline phosphatase, bone sialoprotein, and osteocalcin (Bennett, K. P., et al., BMC Genomics 2007, 8:380, DOI: 10.1186/1471-2164-8-380, Ryoo, H. M., et al., Mol. Endo. 1997, Vol. 11, No. 11, pp. 1681-1694; Hou, Z. et al., Proc. Natl. Acad. Sci., Vol. 96, pp. 7294-7299, June 1999; Engler, A. J., et al., Cell 126, 677-689, Aug. 25, 2006; Marom, R. et al., Journal of Cellular Physiology 202: 41-48 (2005)). Members of the Wnt family also have been shown to impact MSC osteogenesis. Wnts are a family of secreted cysteine-rich glycoproteins that have been implicated in the regulation of stem cell maintenance, proliferation, and differentiation during embryonic development. Canonical Wnt signaling increases the stability of cytoplasmic β-catenin by receptor-mediated inactivation of GSK-3 kinase activity and promotes β-catenin translocation into the nucleus (Liu, G., et al., JCB, Vol. 185, No. 1, 2009, pp. 67-75). The active β-catenin/TCF/LEF complex then regulates the transcription of genes involved in cell proliferation (Novak, A. and Dedhar, S., Cell. Mol. Life Sci. 1999 Oct. 30; 56(5-6); 523-537; Grove, E. A., Genes and Development 2011 25: 1759-1762). In humans, mutations in the Wnt co-receptor, LRP5, lead to defective bone formation (Krishnan, V., et al., The Journal of Clinical Investigation, Vol. 116, No. 5, May 2006, pp. 1202-1209). “Gain of function” mutation results in high bone mass, whereas “loss of function” causes an overall loss of bone mass and strength, indicating that Wnt signaling is positively involved in embryonic osteogenesis (Krishnan, V., et al., The Journal of Clinical Investigation, Vol. 116, No. 5, May 2006, pp. 1202-1209; Niziolek, P. J., et al., Bone 2011 November; 49(5): 1010-1019). Canonical Wnt signaling pathway also functions as a stem cell mitogen via stabilization of intracellular β-catenin and activation of the β-catenin/TCF/LEF transcription complex, resulting in activated expression of cell cycle regulatory genes, such as Myc, cyclin D1, and Msx1 (Willert, J., et al., BMC Development Biology 2002, 2:8, pp. 1-7). When MSCs are exposed to Wnt3a, a prototypic canonical Wnt signal, under standard growth medium conditions, they show markedly increased cell proliferation and a decrease in apoptosis, consistent with the mitogenic role of Wnts in hematopoietic stem cells (Almeida, M., et al., The Journal of Biological Chemistry, Vol. 280, No. 50, pp. 41342-41351, Dec. 16, 2005; Vijayaragavan, K., et al., Cell Stem Cell 4, 248-262, Mar. 6, 2009). However, exposure of MSCs to Wnt3a conditioned medium or overexpression of ectopic Wnt3a during osteogenic differentiation inhibits osteogenesis in vitro through β-catenin mediated down-regulation of TCF activity (Quarto, N., et al., Tissue Engineering: Part A, Vol. 16, No. 10, 2010, pp. 3185-3197). The expression of several osteoblast specific genes, e.g., alkaline phosphatase, bone sialoprotein, and osteocalcin, is dramatically reduced, while the expression of Cbfal/Runx2, an early osteo-inductive transcription factor is not altered, implying that Wnt3a-mediated canonical signaling pathway is necessary, but not sufficient, to completely block MSC osteogenesis (Quarto, N., et al., Tissue Engineering: Part A, Vol. 16, No. 10, 2010, pp. 3185-3197; Eslaminejad, M. B. and Yazdi, P. E., Yakhteh Medical Journal, Vol. 9, No. 3, Autumn 2007, pp. 158-169). On the other hand, Wnt5a, a typical non-canonical Wnt member, has been shown to promote osteogenesis in vitro (Arnsdorf, E. J., et al., PLoS ONE, April 2009, Vol. 4, Issue 4, e5388, pp. 1-10; Baksh, D., et al., J. Cell. Physiol., 2007, 212: 817-826; J. Cell. Biochem., 2007, 101: 1109-1124). Since Wnt3a promotes MSC proliferation during early osteogenesis, it is thought likely that canonical Wnt signaling functions in the initiation of early osteogenic commitment by increasing the number of osteoprecursors in the stem cell compartment, while non-canonical Wnt drives the progression of osteoprecursors to mature functional osteoblasts.

### Soluble Factors

### Hepatocyte Growth Factor/Scatter Factor (HGF/SF)

Hepatocyte growth factor/scatter factor (HGF/SF) is a multifunctional cytokine that promotes mitogenesis, migration, invasion and morphogenesis (Jian, W. G. and S. Hiscox, Histol. Histopathol. 2: 537-555 (1997). HGF/SF signaling modulates integrin function by promoting aggregation and cell adhesion. Morphogenic responses to HGF/SF are dependent on adhesive events. See Matsumoto, K. et al, Cancer Metastasis Rev. 14: 205-217(1995). HGF/SF-induced effects occur via signaling of the MET tyrosine kinase receptor following ligand binding, which leads to enhanced integrin-mediated B cell and lymphoma cell adhesion. Galimi, F. et al, Stem Cells 2: 22-30 (1993); Van der Voort, R. et al., J. Exp. Med. 185: 2121-31 (1997); Weimar, I. S. et al., Blood 89: 990-1000 (1997).

### Tumor Growth Factor (Also Known as Transforming Growth Factor)

The TGF-β1 superfamily of structurally related peptides includes the TGF-β isoforms, β1, β2, β3, and β5, the activins and the bone morphogenetic proteins (BMPs). TGF-β-like factors are a multifunctional set of conserved growth and differentiation factors that control biological processes such as embryogenesis, organogenesis, morphogenesis of tissues like bone and cartilage, vasculogenesis, wound repair and angiogenesis, hematopoiesis, and immune regulation. Signaling by ligands of the TGF-β superfamily is mediated by a high affinity, ligand-induced, heteromeric complex consisting of related Ser/Thr kinase receptors divided into two subfamilies, type I and type II. The type II receptor transphosphorylates and activates the type I receptor in a Gly/Ser-rich region. The type I receptor in turn phosphorylates and transduces signals to a novel family of recently identified downstream targets, termed Smads.

### Osteoprotegerin and RANKL

The molecules osteoprotegerin (OPG) and Receptor activator of NF-κB (RANKL) play a role in the communication between osteoclasts and osteoblasts and are members of a ligand-receptor system that directly regulates osteoclast differentiation and bone resorption. Grimaud, E. et al, Am J. Pathol. 2021-2031 (2993). RANKL has been shown to both activate mature osteoclasts and mediate osteoclastogenesis in the presence of M-CSF, i.e., RANKL is essential for osteoclast differentiation via its receptor RANK located on the osteoclast membrane. OPG is a soluble decoy receptor that prevents RANKL from binding to and activating RANK. It also inhibits the development of osteoclasts and down-regulates the RANKL signaling through RANK. RANKL and OPG have been detected in bone pathological situations where osteolysis occurred. The RANKL/OPG ratio is increased and correlated with markers of bone resorption, osteolytic lesions, and markers of disease activity in multiple myeloma. Id.

### Macrophage Colony-Stimulating Factor (M-CSF)

Macrophage colony-stimulating factor (M-CSF) is a hematopoietic growth factor that is involved in the proliferation, differentiation, and survival of monocytes, macrophages, and bone marrow progenitor cells. Stanley E R, Berg K L, Einstein D B, Lee P S, Pixley F J, Wang Y, Yeung Y G, Mol. Reprod. Dev. 46 (1): 4-10 (1997).

Macrophage inflammatory protein 1-alpha (MIP1α) is a member of the C—C subrfamily of chemokines, a large superfamily of low-molecular weight, inducible proteins that exhibits a variety of proinflammatory activities in vitro. The C—C chemokines generally are chemotactic for cells of the monocyte lineage and lymphocytes. In addition to its proinflammatory activities, MIP-alpha inhibits the proliferation of hematopoietic stem cells in vitro and in vivo. Cook, D. N., J. Leukocyte Biol. 59(1): 61-66 (1996).

### Sclerostin

Sclerostin, a protein expressed by osteocytes, downregulates osteoblastic bone formation by interfering with Wnt signaling.

### Osteogenesis or Ossification

Osteogenesis or ossification is a process by which the bones are formed. There are three distinct lineages that generate the skeleton. The somites generate the axial skeleton, the lateral plate mesoderm generates the limb skeleton, and the cranial neural crest gives rise to the branchial arch, craniofacial bones, and cartilage. There are two major modes of bone formation, or osteogenesis, and both involve the transformation of a preexisting mesenchymal tissue into bone tissue. The direct conversion of mesenchymal tissue into bone is called intramembranous ossification. The process by which mesenchymal cells differentiate into cartilage, which is later replaced by bone cells is called endochondral ossification.

### Intramembranous Ossification

Intramembraneous ossification is the characteristic way in which the flat bones of the scapula, the skull and the turtle shell are formed. In intramembraneous ossification, bones develop sheets of fibrous connective tissue. During intramembranous ossification in the skull, neural crest-derived mesenchymal cells proliferate and condense into compact nodules. Some of these cells develop into capillaries; others change their shape to become osteoblasts, committed bone precursor cells. The osteoblasts secrete a collagen-proteoglycan matrix that is able to bind calcium salts. Through this binding, the prebone (osteoid) matrix becomes calcified. In most cases, osteoblasts are separated from the region of calcification by a layer of the osteoid matrix they secrete. Occasionally, osteoblasts become trapped in the calcified matrix and become osteocytes. As calcification proceeds, bony spicules radiate out from the region where ossification began, the entire region of calcified spicules becomes surrounded by compact mesenchymal cells that form the periosteum, and the cells on the inner surface of the periosteum also become osteoblasts and deposit osteoid matrix parallel to that of the existing spicules. In this manner, many layers of bone are formed.

Intramembraneous ossification is characterized by invasion of capillaries into the mesenchymal zone, and the emergence and differentiation of mesenchymal cells into mature osteoblasts, which constitutively deposit bone matrix leading to the formation of bone spicules, which grow and develop, eventually fusing with other spicules to form trabeculae. As the trabeculae increase in size and number they become interconnected forming woven bone (a disorganized weak structure with a high proportion of osteocytes), which eventually is replaced by more organized, stronger, lamellar bone.

The molecular mechanism of intramembranous ossification involves bone morphogenetic proteins (BMPs) and the activation of a transcription factor called CBFA1. Bone morphogenetic proteins, for example, BMP2, BMP4, and BMP7, from the head epidermis are thought to instruct the neural crest-derived mesenchymal cells to become bone cells directly. BMPs activate the Cbfal gene in mesenchymal cells. The CBFA1 transcription factor is known to transform mesenchymal cells into osteoblasts. Studies have shown that the mRNA for mouse CBFA1 is largely restricted to the mesenchymal condensations that form bone, and is limited to the osteoblast lineage. CBFA1 is known to activate the genes for osteocalcin, osteopontin, and other bone-specific extracellular matrix proteins.

### Endochondral Ossification (Intracartilaginous Ossification)

Endochondral ossification, which involves the in vivo formation of cartilage tissue from aggregated mesenchymal cells, and the subsequent replacement of cartilage tissue by bone, can be divided into five stages. The skeletal components of the vertebral column, the pelvis, and the limbs are first formed of cartilage and later become bone.

First, the mesenchymal cells are committed to become cartilage cells. This commitment is caused by paracrine factors that induce the nearby mesodermal cells to express two transcription factors, Pax1 and Scleraxis. These transcription factors are known to activate cartilage-specific genes. For example, Scleraxis is expressed in the mesenchyme from the sclerotome, in the facial mesenchyme that forms cartilaginous precursors to bone, and in the limb mesenchyme.

During the second phase of endochondral ossification, the committed mesenchyme cells condense into compact nodules and differentiate into chondrocytes (cartilage cells that produce and maintain the cartilaginous matrix, which consists mainly of collagen and proteoglycans). Studies have shown that N-cadherin is important in the initiation of these condensations, and N-CAM is important for maintaining them. In humans, the SOX9 gene, which encodes a DNA-binding protein, is expressed in the precartilaginous condensations.

During the third phase of endochondral ossification, the chondrocytes proliferate rapidly to form the model for bone. As they divide, the chondrocytes secrete a cartilage-specific extracellular matrix.

In the fourth phase, the chondrocytes stop dividing and increase their volume dramatically, becoming hypertrophic chondrocytes. These large chondrocytes alter the matrix they produce (by adding collagen X and more fibronectin) to enable it to become mineralized by calcium carbonate.

The fifth phase involves the invasion of the cartilage model by blood vessels. The hypertrophic chondrocytes die by apoptosis, and this space becomes bone marrow. As the cartilage cells die, a group of cells that have surrounded the cartilage model differentiate into osteoblasts, which begin forming bone matrix on the partially degraded cartilage. Eventually, all the cartilage is replaced by bone. Thus, the cartilage tissue serves as a model for the bone that follows.

The replacement of chondrocytes by bone cells is dependent on the mineralization of the extracellular matrix. A number of events lead to the hypertrophy and mineralization of the chondrocytes, including an initial switch from aerobic to anaerobic respiration, which alters their cell metabolism and mitochondrial energy potential. Hypertrophic chondrocytes secrete numerous small membrane-bound vesicles into the extracellular matrix. These vesicles contain enzymes that are active in the generation of calcium and phosphate ions and initiate the mineralization process within the cartilaginous matrix. The hypertrophic chondrocytes, their metabolism and mitochondrial membranes altered, then die by apoptosis.

In the long bones of many mammals (including humans), endochondral ossification spreads outward in both directions from the center of the bone. As the ossification front nears the ends of the cartilage model, the chondrocytes near the ossification front proliferate prior to undergoing hypertrophy, pushing out the cartilaginous ends of the bone. The cartilaginous areas at the ends of the long bones are called epiphyseal growth plates. These plates contain three regions: a region of chondrocyte proliferation, a region of mature chondrocytes, and a region of hypertrophic chondrocytes. As the inner cartilage hypertrophies and the ossification front extends farther outward, the remaining cartilage in the epiphyseal growth plate proliferates. As long as the epiphyseal growth plates are able to produce chondrocytes, the bone continues to grow.

### Bone Remodeling

Bone constantly is broken down by osteoclasts and re-formed by osteoblasts in the adult. This process of renewal is known as bone remodeling. The balance in this dynamic process shifts as people grow older: in youth, it favors the formation of bone, but in old age, it favors resorption.

As new bone material is added peripherally from the internal surface of the periosteum, there is a hollowing out of the internal region to form the bone marrow cavity. This destruction of bone tissue is due to osteoclasts that enter the bone through the blood vessels. Osteoclasts dissolve both the inorganic and the protein portions of the bone matrix. Each osteoclast extends numerous cellular processes into the matrix and pumps out hydrogen ions onto the surrounding material, thereby acidifying and solubilizing it. The blood vessels also import the blood-forming cells that will reside in the marrow for the duration of the organism's life.

The number and activity of osteoclasts must be tightly regulated. If there are too many active osteoclasts, too much bone will be dissolved, and osteoporosis will result. Conversely, if not enough osteoclasts are produced, the bones are not hollowed out for the marrow, and osteopetrosis (known as stone bone disease, a disorder whereby the bones harden and become denser) will result.

### Lymphocytes and the Immune Response

Multicellular organisms have developed two defense mechanisms to fight infection by pathogens: innate and adaptive immune responses. Innate immune responses are triggered immediately after infection and are independent of the host's prior exposure to the pathogen. Adaptive immune responses operate later in an infection and are highly specific for the pathogen that triggered them. The function of adaptive immune responses is to destroy the invading pathogens and any toxic molecules they produce. (“Chapter 24: The adaptive immune system,” Molecular Biology of the Cell, Alberts, B. et al., Garland Science, NY, 2002).

The immune system consists of a wide range of distinct cell types, amongst which white blood cells called lymphocytes play a central role in determining immune specificity. Other cells, such as monocytes, macrophages, dendritic cells, Langerhans' cells, natural killer (NK) cells, mast cells, basophils, and other members of the myeloid lineage of cells, interact with the lymphocytes and play critical functions in antigen presentation and mediation of immunologic functions. (Paul, W. E., “Chapter 1: The immune system: an introduction,” Fundamental Immunology, 4th Edition, Ed. Paul, W. E., Lippicott-Raven Publishers, Philadelphia (1999)).

Lymphocytes are found in central lymphoid organs, the thymus, and bone marrow, where they undergo developmental steps that enable them to orchestrate immune responses. A large portion of lymphocytes and macrophages comprise a recirculating pool of cells found in the blood and lymph, providing the means to deliver immunocompetent cells to localized sites in need. (Paul, W. E., “Chapter 1: The immune system: an introduction,” Fundamental Immunology, 4th Edition, Ed. Paul, W. E., Lippicott-Raven Publishers, Philadelphia (1999)).

Lymphocytes are specialized cells, committed to respond to a limited set of structurally related antigens. This commitment, which exists before the first contact of the immune system with a given antigen, is expressed by the presence on the lymphocyte's surface of receptors that are specific for specific determinants or epitopes on the antigen. Each lymphocyte possesses a population of cell-surface receptors, all of which have identical combining regions. One set of lymphocyte, referenced to as a “clone” differs from another in the structure of the combining region of its receptors, and thus differs in the epitopes being recognized. The ability of an organism to respond to any nonself antigen is achieved by large number of different clones of lymphocytes, each bearing receptors specific for a distinct epitope. (Paul, W. E., “Chapter 1: The immune system: an introduction,” Fundamental Immunology, 4th Edition, Ed. Paul, W. E., Lippicott-Raven Publishers, Philadelphia (1999)).

The adaptive immune system is composed of millions of lymphocyte clones. The diversity of lymphocytes is such that even a single antigenic determinant is likely to activate many clones, each of which produces an antigen-binding site with its own characteristic affinity for the determinant. Molec. Biol. Of the Cell, 1369). When many clones are activated, such responses are said to be polyclonal; when only a few clones are activated, the response is said to be oligoclonal, and when the response involves only a single B or T cell clone, it is said to be monoclonal.

There are two broad classes of adaptive immune responses that are carried out by different classes of lymphocytes: antibody responses mediated by B-lymphocytes (or B-cells); and cell-mediated immune responses carried out by T-lymphocytes (or T-cells). B-cells are bone-marrow-derived and are precursors of immunoglobulin- (Ig-) or antibody-expressing cells while T-cells are thymus-derived. (Paul, W. E., “Chapter 1: The immune system: an introduction,” Fundamental Immunology, 4th Edition, Ed. Paul, W. E., Lippicott-Raven Publishers, Philadelphia (1999)).

Primary immune responses are initiated by the encounter of an individual with a foreign antigenic substance, generally an infectious microorganism. The infected individual responds with the production of immunoglobulin (Ig) molecules specific for the antigenic determinants of the immunogen and with the expansion and differentiation of antigen-specific regulatory and effector T-lymphocytes. The latter include both T-cells that secrete cytokines as well as natural killer T-cells that are capable of lysing the cell. (Paul, W. E., “Chapter 1: The immune system: an introduction,” Fundamental Immunology, 4th Edition, Ed. Paul, W. E., Lippicott-Raven Publishers, Philadelphia (1999)).

As a consequence of the initial response, the immunized individual develops a state of immunologic memory. If the same (or closely related) microorganism or foreign object is encountered again, a secondary response is triggered. This generally consists of an antibody response that is more rapid and greater in magnitude than the primary (initial) response and is more effective in clearing the microbe from the body. A similar and more effective T-cell response then follows. The initial response often creates a state of immunity such that the individual is protected against a second infection, which forms the basis for immunizations. (Paul, W. E., “Chapter 1: The immune system: an introduction,” Fundamental Immunology, 4th Edition, Ed. Paul, W. E., Lippicott-Raven Publishers, Philadelphia (1999)).

The immune response is highly specific. Primary immunization with a given microorganism evokes antibodies and T-cells that are specific for the antigenic determinants or epitopes found on that microorganism but that usually fail to recognize (or recognize only poorly) antigenic determinants of unrelated microbes. (Paul, W. E., “Chapter 1: The immune system: an introduction,” Fundamental Immunology, 4th Edition, Ed. Paul, W. E., Lippicott-Raven Publishers, Philadelphia (1999)).

### B-Lymphocytes:

B lymphocytes are a population of cells that express clonally diverse cell surface immunoglobulin (Ig) receptors recognizing specific antigenic epitopes.

B-lymphocytes are derived from hematopoietic stem cells by a complex set of differentiation events. The molecular events through which committed early members of the B lineage develop into mature B lymphocytes occur in fetal liver, and in adult life occur principally in the bone marrow. Interaction with specialized stromal cells and their products, including cytokines, such as interleukin IL-7, are critical to the normal regulation of this process. Tucker W. LeBien and Thomas F. Tedder, How they develop and function, Blood 112 (5): 1570-80 (2008). The phenotype of B cells generated with fetal liver is distinct from that using comparable precursors isolated from adult bone marrow. Richard R. Hardy and Kyoko Hayakawa, B Cell Development Pathways, Ann. Rev. Immunol. 19: 595-621 (2001).

Early B-cell development is characterized by the ordered rearrangement of Ig H and L chain loci, and Ig proteins themselves play an active role in regulating B-cell development.

Pre-B cells arise from progenitor (pro-B) cells that express neither the pre-B cell receptor (pre-BCR) or surface immunoglobulin (Ig).

Plasma cells, the critical immune effector cells dedicated to secretion of antigen-specific immunoglobulin (Ig) develop at three distinct stages of antigen-driven B cell development. Short-lived plasma cells emerge in response to both T-independent and T-dependent antigens. TD antigens also induce a germinal center (GC) pathway involving somatic hypermutation, affinity maturation, and production of memory B cells and long-lived PCs. Post-GC PCs have extended half-lives, produce high affinity antibody, and reside preferentially in the bone marrow. Memory B cells rapidly expand and differentiate into PCs in response to antigen challenge. Shapiro-Shelef, et al, Blimp-1 is required for the formation of immunoglobulin secreting plasma cells and pre-plasma memory B cells, Immunity 19: 607-20 (2003)

Antigen-induced B-cell activation and differentiation in secondary lymphoid tissues are mediated by dynamic changes in gene expression that give rise to the germinal center (GC) reaction (see section on B-cell maturation). Tucker W. LeBien and Thomas F. Tedder, How they develop and function, Blood 112 (5): 1570-80 (2008). The GC reaction is characterized by clonal expansion, class switch recombination (CSR) at the IgH locus, somatic hypermutation (SHM) of VH genes, and selection for increased affinity of a BCR for its unique antigenic epitope through affinity maturation.

Lymphocyte development requires the concerted action of a network of cytokines and transcription factors that positively and negatively regulate gene expression. Marrow stromal cell-derived interleukin-7 (IL-7) is a nonredundant cytokine for murine B-cell development that promotes V to DJ rearrangement and transmits survival/proliferation signals.

FLT3-ligand and TSLP play important roles in fetal B-cell development.

The cytokine(s) that regulate human B-cell development are not as well understood, and the cytokine (or cytokines) that promote marrow B-cell development at all stages of human life remains unknown.

At least 10 distinct transcription factors regulate the early stages of B-cell development, with E2A, EBF, and Pax5 being particularly important in promoting B-lineage commitment and differentiation.

Pax5, originally characterized by its capacity to bind to promoter sequences in Ig loci, may be the most multifunctional transcription factor for B cells. Pax5-deficient pro-B cells harbor the capacity to adapt non-B-lineage fates and develop into other hematopoietic lineages (Nutt S L, Heavey B, Rolink A G, Busslinger M., Nature. 1999; 401:556-562). Pax5 also regulates expression of at least 170 genes, a significant number of them important for B-cell signaling, adhesion, and migration of mature B cells (Cobaleda C, Schebesta A, Delogu A, Busslinger M., Nat Immunol. 2007; 8: 463-470). Conditional Pax5 deletion in mature murine B cells can result in dedifferentiation to an uncommitted hematopoietic progenitor and subsequent differentiation into T-lineage cells under certain conditions (Cobaleda C, Jochum W, Busslinger M., Nature. 2007; 449:473-477).

B lymphocyte induced maturation protein (Blimp-1), a transcriptional repressor, a 98 kDa protein containing five zinc finger motifs, has been implicated in plasma cell differentiation, and is required for the complete development of the pre-plasma memory B cell compartment. Shapiro-Shelef, et al, Blilmp-1 is required for the formation of immunoglobulin secreting plasma cells and pre-plasma memory B cells, Immunity 19: 607-20 (2003).

### B Cell Specific Cell Surface Molecules

Table 1 shows Cell surface CD molecules that are preferentially expressed by B cells. Tucker W. LeBien and Thomas F. Tedder, How they develop and function, Blood 112 (5): 1570-80 (2008):

TABLE 1

| Name | Original name | Cellular Reactivity | Structure |
|---|---|---|---|
| CD19 | B4 | Pan-B cell, follicular | Ig superfamily |
| | | dendritic cells | |
| CD20 | B1 | Mature B cells | MS4A family |
| CD21 | B2, HB-5 | Mature B cells, | Complement |
| | | FDCs | receptor family |
| CD22 | BL-CAM, Lyb-8 | Mature B cells | Ig superfamily |
| CD23 | FcεRII | Activated B cells, | C-type lectin |
| | | FDCs, others | |
| CD24 | BA-1, HB-6 | Pen-B cell, | GPI anchored |
| | | granulocytes, | |
| | | epithelial cells | |
| CD40 | Bp50 | B cells, epithelial | TNF receptor |
| | | cells, FDCs, others | |
| CD72 | Lyb-2 | Pam-B cell | C-type lectin |
| CD79 a, b | Igε, β | Surface Ig+ B cells | Ig superfamily |

CD19 is expressed by essentially all B-lineage cells and regulates intracellular signal transduction by amplifying Src-family kinase activity. CD20 is a mature B cell-specific molecule that functions as a membrane-embedded Ca2+ channel. Importantly, ritixumab, the first mAb approved by the Food and Drug Administration (FDA) for clinical use in cancer therapy (eg, follicular lymphoma), is a chimeric CD20 mAb.

CD21 is the C3d and Epstein-Barr virus receptor that interacts with CD19 to generate transmembrane signals and inform the B cell of inflammatory responses within microenvironments.

CD22 functions as a mammalian lectin for α2,6-linked sialic acid that regulates follicular B-cell survival and negatively regulates signaling.

CD23 is a low-affinity receptor for IgE expressed on activated B cells that influences IgE production.

CD24 was among the first pan-B-cell molecules to be identified, but this unique GPI-anchored glycoprotein's function remains unknown.

CD40 serves as a critical survival factor for GC B cells and is the ligand for CD154 expressed by T cells.

CD72 functions as a negative regulator of signal transduction and as the B-cell ligand for Semaphorin 4D (CD100).

There may be other unidentified molecules preferentially expressed by B cells, but the cell surface landscape is likely dominated by molecules shared with multiple leukocyte lineages.

### B-Cell Maturation and Subset Development

Outside the marrow, B cells are morphologically homogenous, but their cell surface phenotypes, anatomic localization, and functional properties reveal still-unfolding complexities. Immature B cells exiting the marrow acquire cell surface IgD as well as CD21 and CD22, with functionally important density changes in other receptors. Immature B cells are also referred to as “transitional” (T1 and T2) based on their phenotypes and ontogeny, and have been characterized primarily in the mouse (Chung J B, Silverman M, Monroe J G., Trends Immunol. 2003; 24:343-349). Immature B cells respond to T cell-independent type 1 antigens such as lipopolysaccharides, which elicit rapid antibody responses in the absence of MHC class II-restricted T-cell help (Coutinho A, Moller G., Adv Immunol. 1975; 21:113-236). The majority of mature B cells outside of the gut associated lymphoid tissue (GALT) reside within lymphoid follicles of the spleen and lymph nodes, where they encounter and respond to T cell-dependent foreign antigens bound to follicular dendritic cells (DCs), proliferate, and either differentiate into plasma cells or enter GC reactions.

Germinal centers (GCs), which refers to sites within lymphoid tissue that are more active in lymphocyte proliferation than are other parts of the lymphoid tissue, containing rapidly proliferating cells (ie, centroblasts) are the main site for high-affinity antibody-secreting plasma cell and memory B-cell generatior (Jacob J, Kelsoe G, Rajewsky K, Weiss U., Nature. 1991; 354:389-392). Within GCs, somatic hypermutation (SHM) and purifying selection produce the higher affinity B-cell clones that form the memory compartments of humoral immunity (Jacob J, Kelsoe G, Rajewsky K, Weiss U., Nature. 1991; 354:389-392; Kelsoe G., Immunity. 1996; 4:107-111). Affinity maturation in GCs does not represent an intrinsic requirement for BCR signal strength but rather a local, Darwinian competition. The dynamics of lymphocyte entry into follicles and their selection for migration into and within GCs represents a complex ballet of molecular interactions orchestrated by chemotactic gradients and B-cell receptor (BCR) engagement that is only now being elucidated (Allen C D, Okada T, Cyster J G., Immunity. 2007; 27:190-202).

B-cell subsets with individualized functions such as B-1 and marginal zone (MZ, referring to the junction of the lymphoid tissue of a lymphatic nodule with the surrounding non-lymphoid red pulp of the spleen) B cells have also been identified. Murine B-1 cells are a unique CD5+ B-cell subpopulation (Hayakawa K, Hardy R R, Parks D R, Herzenberg L A., J Exp Med. 1983; 157:202-218) distinguished from conventional B (B-2) cells by their phenotype, anatomic localization, self-renewing capacity, and production of natural antibodies (Hardy R R, Hayakawa K., Annu Rev Immunol. 2001; 19:595-621). Peritoneal B-1 cells are further subdivided into the B-1a (CD5+) and B-1b (CD5−) subsets. Their origins, and whether they derive from the same or distinct progenitors compared with B-2 cells, have been controversial (Dorshkind K, Montecino-Rodriguez E., Nat Rev Immunol. 2007; 7:213-219). However, a B-1 progenitor that appears distinct from a B-lineage progenitor that develops primarily into the B-2 population has been identified in murine fetal marrow, and to a lesser degree in adult marrow (Montecino-Rodriguez E, Leathers H, Dorshkind K., Nat Immunol. 2006; 7:293-301). B-1a cells and their natural antibody products provide innate protection against bacterial infections in naive hosts, while B-1b cells function independently as the primary source of long-term adaptive antibody responses to polysaccharides and other T cell-independent type 2 antigens during infection (Montecino-Rodriguez E, Leathers H, Dorshkind K., Nat Immunol. 2006; 7:293-301). The function and potential subpopulation status of human B-1 cells is less understood (Dorshkind K, Montecino-Rodriguez E., Nat Rev Immunol. 2007; 7:213-219). MZ B cells are a unique population of murine splenic B cells with attributes of naive and memory B cells (Pillai S, Cariappa A, Moran S T., Annu Rev Immunol. 2005; 23:161-196), and constitute a first line of defense against blood-borne encapsulated bacteria. Uncertainty regarding the identity of human MZ B cells partially reflects the fact that the microscopic anatomy of the human splenic MZ differs from rodents (Steiniger B, Timphus E M, Barth P J., Histochem Cell Biol. 2006; 126:641-648). Likewise, the microscopic anatomy of human follicular mantle zones is not recapitulated in mouse spleen and lymph nodes.

The B1, MZ, and GC B-cell subsets all contribute to the circulating natural antibody pool, thymic-independent IgM antibody responses, and adaptive immunity by terminal differentiation into plasma cells, the effector cells of humoral immunity (Radbruch A, Muehlinghaus G, Luger E O, et al., Nat Rev Immunol. 2006; 6:741-750). Antigen activation of mature B cells leads initially to GC development, the transient generation of plasmablasts that secrete antibody while still dividing, and short-lived extrafollicular plasma cells that secrete antigen-specific germ line-encoded antibodies (FIG. 1). GC-derived memory B cells generated during the second week of primary antibody responses express mutated BCRs with enhanced affinities, the product of SHM. Memory B cells persist after antigen challenge, rapidly expand during secondary responses, and can terminally differentiate into antibody-secreting plasma cells. In a manner similar to the early stages of B-cell development in fetal liver and adult marrow, plasma cell development is tightly regulated by a panoply of transcription factors, most notably Bcl-6 and BLIMP-1 (Shapiro-Shelef M, Calame K., Nat Rev Immunol. 2005; 5:230-242).

Persistent antigen-specific antibody titers derive primarily from long-lived plasma cells (Radbruch A, Muehlinghaus G, Luger E O, et al., Nat Rev Immunol. 2006; 6:741-750). Primary and secondary immune responses generate separate pools of long-lived plasma cells in the spleen, which migrate to the marrow where they occupy essential survival niches and can persist for the life of the animal without the need for self-replenishment or turnover ((Radbruch A, Muehlinghaus G, Luger E O, et al., Nat Rev Immunol. 2006; 6:741-750; McHeyzer-Williams L J, McHeyzer-Williams M G., Annu Rev Immunol. 2005; 23:487-513). The marrow plasma cell pool does not require ongoing contributions from the memory B-cell pool for its maintenance, but when depleted, plasma cells are replenished from the pool of memory B cells (Dilillo D J, Hamaguchi Y, Ueda Y, et al., J Immunol. 2008; 180:361-371). Thereby, persisting antigen, cytokines, or Toll-like receptor signals may drive the memory B-cell pool to chronically differentiate into long-lived plasma cells for long-lived antibody production.

In addition to their essential role in humoral immunity, B cells also mediate/regulate many other functions essential for immune homeostasis. Of major importance, B cells are required for the initiation of T-cell immune responses, as first demonstrated in mice depleted of B cells at birth using anti-IgM antiserum (Ron Y, De Baetselier P, Gordon J, Feldman M, Segal S., Eur J Immunol. 1981; 11:964-968). However, this has not been without controversy as an absence of B cells impairs CD4 T-cell priming in some studies, but not others. Nonetheless, antigen-specific interactions between B and T cells may require the antigen to be first internalized by the BCR, processed, and then presented in an MHC-restricted manner to T cells (Ron Y, Sprent J., J Immunol. 1987; 138:2848-2856; Janeway C A, Ron J, Jr, Katz M E., J Immunol. 1987; 138:1051-1055; Lanzavecchia A., Nature. 1985; 314:537-539).

### B-Cell Abnormalities

The normal B-cell developmental stages have malignant counterparts that reflect the expansion of a dominant subclone leading to development of leukemia and lymphoma.

For example, non-T, non-B ALL is a malignancy of B-cell precursors (Korsmeyer S J, Arnold A, Bakhshi A, et al., J Clin Invest. 1983; 71:301-313). The antiapoptotic Bcl-2 gene was discovered as the translocation partner with the IgH locus in the t(14;18)(q32;q21); frequently occurring in follicular lymphoma (Tsujimoto Y, Finger L R, Yunis J, Nowell P C, Croce C M., Science. 1984; 226:1097-1099). A substantial number of cases of diffuse large B-cell lymphoma exhibit dysregulated expression of the transcriptional repressor Bcl-6 (Ye B H, Lista F, Lo Coco F, et al., Science. 1993; 262:747-750). The Hodgkin/Reed-Sternberg cell in Hodgkin lymphoma, is of B-lymphocyte origin based on the demonstration of clonal Ig gene rearrangements (Kuppers R, Rajewsky K, Zhao M, et al., Proc Natl Acad Sci USA. 1994; 91: 10962-10966).

The monoclonal gammopathies (paraproteinemias or dysproteinemias) are a group of disorders characterized by the proliferation of a single clone of plasma cells which produces an immunologically homogeneous protein commonly referred to as a paraprotein or monoclonal protein (M-protein, where the “M” stands for monoclonal). Each serum M-protein consists of two heavy polypeptide chains of the same class designated by a capital letter and a corresponding Greek letters: Gamma (γ) in IgG, Alpha (α) in IgA, Mu (μ) in IgM, Delta (δ) in IgD, Epsilon (ε) in IgE. For example, basophils in IgE myeloma are characterized by a higher expression of high affinity IgE receptor relative to normal controls.

Multiple Myeloma

Multiple myeloma (MM), a B cell malignancy characterized by the accumulation of plasma cells in the BM and the secretion of large amounts of monoclonal antibodies that ultimately causes bone lesions, hypercalcaemia, renal disease, anemia, and immunodeficiency (Raab M S, Podar K, Breitkreutz I, Richardson P G, Anderson K C., Lancet 2009; 374:324-39), is the second most frequent blood disease in the United States affecting 7.1 per 100,000 men and 4.6 per 100,000 women.

MM is characterized by monoclonal proliferation of malignant plasma cells (PCs) in the bone marrow (BM), the presence of high levels of monoclonal serum antibody, the development of osteolytic bone lesions, and the induction of angiogenesis, neutropenia, amyloidosis, and hypercalcemia (Vanderkerken K, Asosingh K, Croucher P, Van Camp B., Immunol Rev 2003; 194:196-206; Raab M S, Podar K, Breitkreutz I, Richardson P G, Anderson K C., Lancet 2009; 374:324-39). MM is seen as a multistep transformation process. G. Pratt., Molecular Aspects of multiple myeloma, J. Clin. Pathol: Molec. Pathol. 55: 273-83 (2002). Although little is known about the immortalizing and initial transforming events, the initial event is thought to be the immortalization of a plasma cell to form a clone, which may be quiescent, non-accumulating and not cause end organ damage due to accumulation of plasma cells within the bone marrow (MGUS). Smouldering MM (SMM) also has no detectable end-organ damage, but differs from MGUS by having a serum mIg level higher than 3 g/dl or a BM PC content of more than 10% and an average rate of progression to symptomatic MM of 10% per year. Currently there are no tests that measure phenotypic or genotypic markers on tumor cells that predict progression. W. Michael Kuehl and P. Leif Bergsagel, Molecular pathogenesis of multiple myeloma and its premalignant precursor, J. Clin. Invest. 122 (10): 3456-63 (2012). An abnormal immunophenotype distinguishes healthy plasma cells (PCs) from tumor cells. Healthy BM PCs are CD38+CD138+CD19+CD45+CD56−. Id. Although MM tumor cells also are CD38+CD138+, 90% are CD19−, 99% are CD45− or CD45 lo, and 70% are CD56+. Id.

The prognosis and treatment of this disease has greatly evolved over the past decade due to the incorporation of new agents that act as immunomodulators and proteosome inhibitors. Despite recent progress with a number of novel treatments (Raab M S, Podar K, Breitkreutz I, Richardson P G, Anderson K C., Lancet 2009; 374:324-39; Schwartz R N, Vozniak M., J Manag Care Pharm 2008; 14:12-9), patients only experience somewhat longer periods of remission. Because of the development of drug resistance or relapse, MM is an incurable disease (Schwartz R N, Vozniak M., J Manag Care Pharm 2008; 14:12-9; Kyle R A., Blood 2008; 111:4417-8), with a median survival time of 3-4 years.

Disease management is currently tailored based on the patient's co-morbidity factors and stage of disease (for a complete list of treatments and their implementation, see Raab M S, Podar K, Breitkreutz I, Richardson P G, Anderson K C., Lancet 2009; 374:324-39 and Schwartz R N, Vozniak M., J Manag Care Pharm 2008; 14:12-9).

Allogeneic blood and marrow transplantation (allo-BMT) is an effective immunotherapeutic treatment that can provide partial or complete remission for patients with drug-resistant hematological malignancies, including multiple myeloma.

Staging of Myeloma

While multiple myeloma may be staged using the Durie-Salmon system, its value is becoming limited because of newer diagnostic methods. The International Staging System for Multiple Myeloma relies mainly on levels of albumin and beta-2-microglobulin in the blood. Other factors that may be important are kidney function, platelet count and the patient's age. [www.cancer.org/cancer/multiplemyeloma/detailedguide/multiple-myeloma-staging, last revised Feb. 12, 2013]

The Durie-Salmon staging system is based on 4 factors:

The amount of abnormal monoclonal immunoglobulin in the blood or urine: Large amounts of monoclonal immunoglobulin indicate that many malignant plasma cells are present and are producing that abnormal protein.

The amount of calcium in the blood: High blood calcium levels can be related to advanced bone damage. Because bone normally contains lots of calcium, bone destruction releases calcium into the blood.

The severity of bone damage based on x-rays: Multiple areas of bone damage seen on x-rays indicate an advanced stage of multiple myeloma.

The amount of hemoglobin in the blood: Hemoglobin carries oxygen in red blood cells. Low hemoglobin levels mean that the patient is anemic; it can indicate that the myeloma cells occupy much of the bone marrow and that not enough space is left for the normal marrow cells to make enough red blood cells.

This system uses these factors to divide myeloma into 3 stages. Stage I indicates the smallest amount of tumor, and stage III indicates the largest amount of tumor:

In Stage I, a relatively small number of myeloma cells are found. All of the following features must be present:

Hemoglobin level is only slightly below normal (still above 10 g/dL)

Bone x-rays appear normal or show only 1 area of bone damage

Calcium levels in the blood are normal (less than 12 mg/dL)

Only a relatively small amount of monoclonal immunoglobulin is in blood or urine

In Stage II, a moderate number of myeloma cells are present. Features are between stage I and stage III.

In Stage III, a large number of myeloma cells are found. One or more of the following features must be present:

Low hemoglobin level (below 8.5 g/dL)

High blood calcium level (above 12 mg/dL)

3 or more areas of bone destroyed by the cancer

Large amount of monoclonal immunoglobulin in blood or urine

The International Staging System divides myeloma into 3 stages based only on the serum beta-2 microglobulin and serum albumin levels.

In Stage I, serum beta-2 microglobulin is less than 3.5 (mg/L) and the albumin level is above 3.5 (g/L). Stage II is neither stage I nor III, meaning that either: The beta-2 microglobulin level is between 3.5 and 5.5 (with any albumin level), OR the albumin is below 3.5 while the beta-2 microglobulin is less than 3.5. In Stage III, Serum beta-2 microglobulin is greater than 5.5.

Factors other than stage that affect survival include kidney function (when the kidneys are damaged by the monoclonal immunoglobulin, blood creatinine levels rise, predicting a worse outlook); age (in the studies of the international staging system, older people with myeloma do not live as long); the myeloma labeling index (sometimes called the plasma cell labeling index), which, indicates how fast the cancer cells are growing; a high labeling index can predict a more rapid accumulation of cancer cells and a worse outlook; and chromosome studies, i.e., certain chromosome changes in the malignant cells can indicate a poorer outlook. For example, changes in chromosome 13 will lower a person's chances for survival. Another genetic abnormality that predicts a poor outcome is a translocation (meaning an exchange of material) from chromosomes 4 and 14.

Biological pharmacotherapy for the treatment of MM currently includes immunomodulatory agents, such as thalidomide or its analogue, lenalidomide, and bortezomib, a first-in-class proteosome inhibitor. Unfortunately, some side effects associated with these therapies such as peripheral neuropathy and thrombocytopenia (in the case of bortezomib) restrict dosing and duration of treatment (Raab M S, Podar K, Breitkreutz I, Richardson P G, Anderson K C., Lancet 2009; 374:324-39; Schwartz R N, Vozniak M., J Manag Care Pharm 2008; 14:12-9; Field-Smith A, Morgan G J, Davies F E., Ther Clin Risk Manag 2006; 2:271-9).

Despite significant advances in the implementation of these drugs, MM still remains a lethal disease for the vast majority of patients. Since MM is a disease characterized by multiple relapses, the order/sequencing of the different effective treatment options is crucial to the outcome of MM patients. In the frontline setting, the first remission is likely to be the period during which patients will enjoy the best quality of life. Thus, one goal is to achieve a first remission that is the longest possible by using the most effective treatment upfront. At relapse, the challenge is to select the optimal treatment for each patient while balancing efficacy and toxicity. The decision will depend on both disease- and patient-related factors (Mohty B, El-Cheikh J, Yakoub-Agha I, Avet-Loiseau H, Moreau P, Mohty M., Leukemia 2012; 26:73-85). Thus, having the capability of testing the efficacy of a potential therapy, prior to patient treatment, can have a major impact in the management of this disease.

As opposed to other hematological malignancies, MM as well as other cancers that metastasize to the BM strongly interact with the BM microenvironment, which is composed of endothelial cells, stromal cells, osteoclasts (OCL), osteoblasts (OSB), immune cells, fat cells and the extracellular matrix (ECM). These interactions, as illustrated in FIG. 1 (adapted from Roodman G D., Bone 2011; 48:135-40), are responsible for the specific homing in the BM, the proliferation and survival of the MM cells, the resistance of MM cells to drug treatment, and the development of osteolysis, immunodeficiency, and anemia (Dvorak H F, Weaver V M, Tlsty T D, Bergers G., J Surg Oncol 2011; 103:468-74; De Raeve H R, Vanderkerken K., Histol Histopathol 2005; 20:1227-50; Fowler J A, Edwards C M, Croucher P I., Bone 2011; 48:121-8; Fowler J A, Mundy G R, Lwin S T, Edwards C M., Cancer Res 2012; Roodman G D., J Bone Miner Res 2002; 17:1921-5).

The Bone Marrow Niche and MM Progression

The BM niche plays a key role in MM-related bone disease. A complex interaction with the BM microenvironment in areas adjacent to tumor foci, characterized by activation of osteoclasts and suppression of osteoblasts, leads to lytic bone disease. W. Michael Kuehl and P. Leif Bergsagel, Molecular pathogenesis of multiple myeloma and its premalignant precursor, J. Clin. Invest. 122 (10): 3456-63 (2012); Shmuel Yaccoby, Advanaces in the understanding of myeloma bone disease and tumour growth, Br. J. Haematol. 149 (3): 311-321 (2010). Thus, although the MM microenvironment is highly complex, it is understood that suppression of OSB activity plays a key role in the bone destructive process as well as progression of the tumor burden (Roodman G D., Bone 2011; 48:135-40). Treatments that target both the bone microenvironment as well as the tumor, such as bortezomib and immunomodulatory drugs, have been more effective than prior therapies for MM and have dramatically increased both progression-free survival and overall survival of patients.

MM cells closely interact with the BM microenvironment, also termed the cancer niche. The elements of the bone marrow niche can provide an optimal growth environment for multiple hematological malignancies including multiple myeloma (MM). MM cells convert the bone marrow into specialized neoplastic niche, which aids the growth and spreading of tumor cells by a complex interplay of cytokines, chemokines, proteolytic enzymes and adhesion molecules. Moreover, the MM BM microenvironment confers survival and chemoresistance of MM cells to current therapies.

Bone Marrow Stromal Cells (BMSCs)

Multiple myeloma (MM) cells adhere to BMSC and ECM. Tumor cells, such as MM cells, bind to ECM proteins, such as type I collagen and fibronectin via syndecan 1 and very late antigen 4 (VLA-4) on MM cells and to BMSC VCAM-1 via VLA-4 on MM cells. Adhesion of MM cells to BMSC activates many pathways resulting in upregulation of cell cycle regulating proteins and antiapoptotic proteins (Hideshima T, Bergsagel P L, Kuehl W M, Anderson K C., Blood. 2004; 104(3):607-618). The interaction between MM cells and BMSCs triggers NF-κB signaling pathway and interleukin-6 (IL-6) secretion in BMSCs. In turn, IL-6 enhances the production and secretion of VEGF by MM cells. The existence of this paracrine loop optimizes the BM milieu for MM tumor cell growth (Kumar S, Witzig T E, Timm M, et al., Leukemia. 2003; 17(10):2025-2031). BMSC-MM cell interaction is also mediated through Notch. The Notch-signaling pathways both in MM cells as well as in BMSC, promote the induction of IL-6, vascular endothelial growth factor (VEGF), and insulin-like growth factor (IGF-1) secretion and is associated with MM cell proliferation and survival (Radtke F, Raj K., Nature Reviews Cancer. 2003; 3(10):756-767; Nefedova Y, Cheng P, Alsina M, Dalton W S, Gabrilovich D I., Blood. 2004; 103(9):3503-3510). It has been shown that BMSC from MM patients expresses several proangiogenic molecules, such as VEGF, basic-fibroblast growth factor (bFGF), angiopoietin 1 (Ang-1), transforming growth factor (TGF)-β, platelet-derived growth factor (PDGF), hepatocyte growth factor (HGF) and interleukin-1 (IL-1) (Giuliani N, Storti P, Bolzoni M, Palma B D, Bonomini S., Cancer Microenvironment. 2011; 4(3):325-337). BMSCs from MM patients also have been shown to release exosomes, which are transferred to MM cells, thereby resulting in modulation of tumor growth in vivo, mediated by specific miRNA (Roccaro A M, Sacco A, Azab A K, et al., Blood. 2011; 118, abstract 625 ASH Annual Meeting Abstracts).

Endothelial Cells and Angiogenesis

BM angiogenesis represents a constant hallmark of MM progression, partly driven by release of pro-angiogenic cytokines from the tumor plasma cells, BMSC, and osteoclasts, such as VEGF, bFGF, and metalloproteinases (MMPs). The adhesion between MM cells and BMSCs upregulates many cytokines with angiogenic activity, most notably VEGF and bFGF (Podar K, Anderson K C., Blood. 2005; 105(4):1383-1395). In MM cells, these pro-angiogenic factors may also be produced constitutively as a result of oncogene activation and/or genetic mutations (Rajkumar S V, Witzig T E., Cancer Treatment Reviews. 2000; 26(5):351-362). Evidence for the importance of angiogenesis in the pathogenesis of MM was obtained from BM samples from MM patients (Kumar S, Gertz M A, Dispenzieri A, et al., Bone Marrow Transplantation. 2004; 34(3):235-239). The level of BM angiogenesis, as assessed by grading and/or microvessel density (MVD), is increased in patients with active MM as compared to those with inactive disease or monoclonal gammopathy of undetermined significance (MGUS), a less advanced plasma cell disorder. Comparative gene expression profiling of multiple myeloma endothelial cells and MGUS endothelial cells has been performed in order to determine a genetic signature and to identify vascular mechanisms governing the malignant progression (Ria R, Todoerti K, Berardi S, et al., Clinical Cancer Research. 2009; 15(17):5369-5378). Twenty-two genes were found differentially expressed at relatively high stringency in MM endothelial cells compared with MGUS endothelial cells. Functional annotation revealed a role of these genes in the regulation of ECM formation and bone remodelling, cell adhesion, chemotaxis, angiogenesis, resistance to apoptosis, and cell-cycle regulation. The distinct endothelial cell gene expression profiles and vascular phenotypes detected may influence remodelling of the bone marrow microenvironment in patients with active multiple myeloma. Overall, these evidences suggest that EC presents with functional, genetic, and morphologic features indicating their ability to induce BM neovascularization, resulting in MM cell growth, and disease progression.

Osteoclasts

The usual balance between bone resorption and new bone formation is lost in many cases of MM, resulting in bone destruction and the development of osteolytic lesions (Bataille R, Chappard D, Marcelli C, et al., Journal of Clinical Oncology. 1989; 7(12):1909-1914). Bone destruction develops adjacent to MM cells, yet not in areas of normal bone marrow. There are several factors implicated in osteoclast activation, including receptor activator of NF-κB ligand (RANKL), macrophage inflammatory protein-1a (MIP-1a), interleukin-3 (IL-3), and IL-6 (Roodman G D., Leukemia. 2009; 23(3):435-441). RANK ligand is a member of the tumor necrosis factor (TNF) family and plays a major role in the increased osteoclastogenesis implicated in MM bone disease. RANK is a transmembrane signaling receptor expressed by osteoclast cells. MM cell binding to neighboring BMSC within the bone marrow results in increased RANKL expression. This leads to an increase in osteoclast activity through the binding of RANKL to its receptor, on osteoclast precursor cells, which further promotes their differentiation through NF-κB and JunN-terminal kinase pathway (Ehrlich L A, Roodman G D., Immunological Reviews. 2005; 208:252-266). RANKL is also involved in inhibition of osteoclast apoptosis. Blocking RANKL with a soluble form of RANK has been shown to modulate not only bone loss but also tumor burden in MM in vivo models (Yaccoby S, Pearse R N, Johnson C L, Barlogie B, Choi Y, Epstein J., British Journal of Haematology. 2002; 116(2):278-290). Moreover osteoclasts constitutively secrete proangiogenic factors osteopontin that enhanced vascular tubule formation (Tanaka Y, Abe M, Hiasa M, et al., Clinical Cancer Research. 2007; 13(3):816-823).

Osteoblasts in MM Progression

Osteoblasts are thought to contribute to MM pathogenesis by supporting MM cells growth and survival (Karadag A, Oyajobi B O, Apperley J F, Graham R, Russell G, Croucher P I., British Journal of Haematology. 2000; 108(2):383-390). This could potentially result from the ability of osteoblasts to secrete IL-6 in a co-culture system with MM cells, thus increasing IL-6 levels within the BM milieu and inducing MM plasma cell growth. Other mechanisms include the possible role of osteoblasts in stimulating MM cell survival by blocking TRAIL-mediated programmed MM cell death, by secreting osteoprotegerin (OPG), a receptor for both RANKL and TRAIL (Shipman C M, Croucher P I., Cancer Research. 2003; 63(5):912-916). In addition, suppression of osteoblast activity is responsible for both bone destructive process and progression of myeloma tumor burden. Several factors have been implicated in the suppression of osteoblast activity in MM, including DKK1 (Tian E, Zhan F, Walker R, et al., The New England Journal of Medicine. 2003; 349(26):2483-2494). DKK1 is a Wnt-signaling antagonist secreted by MM cells that inhibits osteoblast differentiation. DKK1 is significantly overexpressed in patients with MM who present with lytic bone lesions. Myeloma-derived DKK1 also disrupts Wnt-regulated OPG and RANKL production by osteoblasts. Studies have shown that blocking DKK1 and activating Wnt signaling prevents bone disease in MM and is associated with a reduction in tumor burden (Yaccoby S, Ling W, Zhan F, Walker R, Barlogie B, Shaughnessy J D., Jr., Blood. 2007; 109(5):2106-2111; Edwards C M, Edwards J R, Lwin S T, et al., Blood. 2008; 111(5):2833-2842; Fulciniti M, Tassone P, Hideshima T, et al., Blood. 2009; 114(2):371-379).

Many components of the microenvironment support the propagation of the MM cells through cell-cell adhesion and the release of growth factors such as interleukin-6 (IL-6) and insulin-like growth factor-1 (IGF-1) (Deleu S, Lemaire M, Arts J, et al., Leukemia 2009; 23:1894-903; Field-Smith A, Morgan G J, Davies F E., Ther Clin Risk Manag 2006; 2:271-9; D'Souza S, del Prete D, Jin S, et al. Blood 2011; 118:6871-80). Survival and drug resistance of malignant cells is associated with their ability to shape the local microenvironment, in part by disrupting the balance of pro- and anti-angiogenic factors through neovascularization (Otjacques E, Binsfeld M, Noel A, Beguin Y, Cataldo D, Caers J., Int J Hematol 2011; 94:505-18) and bone remodeling which leads to osteolysis (Raje N, Roodman G D., Clin Cancer Res 2011; 17:1278-86; Giuliani N, Rizzoli V, Roodman G D., Blood 2006; 108:3992-6; Lentzsch S, Ehrlich L A, Roodman G D., Hematol Oncol Clin North Am 2007; 21:1035-49, viii).

Unfortunately, primary MM tumor cells have been difficult to propagate ex vivo because they require a microenvironment hard to reproduce in vitro. MM cells grown in vitro therefore are very short lived and grow poorly outside their BM milieu and attempts to optimize their maintenance have been hampered by lack of known conditions that allow for their ex vivo survival (Zlei M, Egert S, Wider D, Ihorst G, Wasch R, Engelhardt M., Exp Hematol 2007; 35:1550-61). Aside from various xenograft models (Calimeri T, Battista E, Conforti F, et al., Leukemia 2011; 25:707-11; Yata K, Yaccoby S., Leukemia 2004; 18:1891-7; Yaccoby S, Johnson C L, Mahaffey S C, Wezeman M J, Barlogie B, Epstein J., Blood 2002; 100:4162-8; Bell E., Nature Reviews Immunology 2006; 6:87), only one group to date has reported on creating an in vitro model capable of supporting the proliferation and survival of MM cells (Kirshner J, Thulien K J, Martin L D, et al., Blood 2008; 112:2935-45). However, the macroscale static methodology that was employed has limited value as, inter alia, it fails to recapitulate the spatial and temporal characteristics of the complex tumor niche.

Although microphysiologically relevant human three dimensional (3D) tissue and tumor models cannot replicate the biological and physiological complexity associated with homeostatic and disease progressions that occur over a long period of time, such models may provide “snapshot” ex vivo reproductions of authentic phenotypic cell functions and interactions relating to specific persons and disease states.

It is well recognized that serially cultured human diploid cells have a finite lifetime in vitro. Hayflick, L. Exptl Cell Res. 37: 614-636 (1965). After a period of active multiplication, generally less than one year, these cells demonstrate an increased generation time, gradual cessation of mitotic activity, accumulation of cellular debris, and, ultimately, total degeneration of the culture. Id. However, conventional practices of immortalizing human cells into cell lines by gene transfection perturbs the cells' gene expression profiles, cellular physiology and physical integrity of their genome. Even if primary cells can be grown and maintained, gene expression and cellular physiology of such cells can be fundamentally different in 2D versus 3D culture environments.

A three-dimensional (3D) tissue construct in which a polydimethylsiloxane (PDMS) multichannel microfluidic device was used to create mineralized 3D tissue-like structures by dynamic long-term culture of osteoblasts, which formed a confluent layer on the bottom channel surface, gradually migrated to the side and top surfaces, and formed calcified 3D nodular structures in 8 days was described. This 3D ossified tissue has been used to create a microfluidic 3D MM/bone tissue model.

The described invention provides a multiwell plate-based perfusion tissue cell culture device that provides a perfused microenvironment, facilitates the seeding of adherent and non-adherent BM cells, and accelerates reconstruction of the BM milieu. The model system preserves BM/MM interactions, and, from a clinical perspective, enables a physiologically relevant system that: 1) maximizes sample use by requiring very small amounts of patient BM cells (<1×10⁶ cells) and plasma (<2 mL/culture/week) and 2) accelerates the evaluation of new therapeutics for the treatment of MM. Furthermore, because real-time monitoring of BM/MM cell developments and interactions are performed, the described model is useful to study and identify new mechanisms associated with the MM niche and tumor progression.

### SUMMARY OF THE INVENTION

According to one aspect, the described invention provides an ex vivo model of a three dimensional (3D) cellular network found in native bones via biomimetic assembly of osteocytes and microbeads in a microfluidic perfusion culture device comprising (a) preparing an in vitro multiwell plate-based perfusion culture device, comprising, from top to bottom: 1. a bottomless multi-well plate comprising a plurality of bottomless wells; 2. a first micropatterned polymer layer attached to a bottom surface of the bottomless multi-well plate to form a plurality of adjacent wells, one or more of each pair of adjacent wells comprising a transparent polymer membrane placed within the one or more of each pair of adjacent wells; 3. a second micropatterned polymer layer comprising two or more holes that correspond to two or more adjacent wells, the second micropatterned polymer layer being attached to a bottom surface of the first micropatterned polymer layer, such that each hole of the second micropatterned polymer layer is aligned with the two or more adjacent wells in the first micropatterned polymer layer, one or more of each pair of adjacent wells comprising the transparent polymer membrane; 4. a microfluidic channel formed between the two adjacent wells that allows internal fluidic communication between the two adjacent wells; 5. one or more removable polymer plugs, each located at a top surface of each of the plurality of wells, and one or more tubes, each connected to the one or more polymer plugs; 6. a pump connected to a reservoir that removably connects to the tubes; 7. a transparent, optical grade glass layer attached to the bottom surface of the second micropatterned polymer layer that forms a bottom surface for the plurality of wells and that seals the multi-well plate perfusion culture device; wherein (i) one or more of the two adjacent wells is a cell culture chamber comprising a first well region including a first well and a second well region including a second well; (ii) the microfluidic channel connects the first well region and the second well region with one another; (iii) the first well is adapted to receive a therapeutic agent, the second well is adapted to receive a biological sample of cells; and (iv) liquids, nutrients and dissolved gas molecules flow through the channel (b) constructing an ex vivo endosteal microenvironment perfused by nutrients and dissolved gas molecules by: 1. seeding a surface of the culture chamber of the device of (a) with (i) microbeads; (ii) osteocyte cells (OSTs); and (iii) osteoblast cells (OSBs), and 2. culturing the cells with a culture medium through the microfluidic channel for a time effective for the cells to form three-dimensional (3D) nodular structures that comprise a 3D-endosteal-like tissue.

According to one embodiment, the described invention provides a method for selecting a patient-specific treatment for multiple myeloma (MM) comprising: (a) preparing the ex vivo endosteal microenvironment perfused by nutrients and dissolved gas molecules comprising three-dimensional (3D) nodular structures that comprise a 3D-endosteal-like tissue as described above; (b) acquiring bone marrow mononuclear cells (BMMCs) comprising viable multiple myeloma cells (MMCs) from a subject; (c) bringing the BMMCs comprising viable MMCs in contact with the endosteal microenvironment perfused by nutrients and gas molecules to seed the ex vivo endosteal microenvironment with the viable MMCs, the ex vivo endosteal microenvironment perfused by nutrients and gas molecules seeded with viable MMCs forming an ex vivo microenvironment effective to recapitulate spatial and temporal characteristics of a multiple myeloma cancer niche and to maintain viability of the MMCs from the subject; and (d) testing therapeutic efficacy of a therapeutic agent on the viable MMCs maintained by the endosteal microenvironment in the first well adapted to receive a therapeutic agent by 1. contacting the MMCs maintained by the endosteal microenvironment of (d) with a test therapeutic agent; and 2. comparing at least one of viability and level of apoptosis of the MMCs contacted with the test therapeutic agent to an untreated MMC control, and (e) initiating therapy to treat the subject with the test therapeutic agent if the test therapeutic agent is effective to significantly reduce viability of the MMCs contacted with the test therapeutic agent or to increase apoptosis of the MMCs contacted with the test therapeutic agent compared to the untreated MMC control.

According to another embodiment, the microbeads are biphasic calcium phosphate (BCP) microbeads, polystyrene (PS) microbeads or a combination thereof. According to another embodiment, the microbeads range in diameter from about 20 μm to about 25 μm. According to another embodiment, the osteocyte cells are primary human osteocytes (ph-OSTs) or murine osteocytes. According to another embodiment, the osteoblast cells (OSBs) are primary human osteoblasts (ph-OSBs). According to another embodiment, the primary human osteoblasts (ph-OSBs) are autologous ph-OSBs. According to another embodiment, the gas molecules are oxygen (O2) molecules.

According to one embodiment of the method, the therapeutic agent is selected from the group consisting of a chemotherapeutic agent, a corticosteroid, an immunomodulating agent, a proteasome inhibitor, a histone deacetylase (HDAC) inhibitor, a monoclonal antibody and interferon. According to another embodiment, the chemotherapeutic agent is selected from the group consisting of melphalan, vincristine, cyclophosphamide, etoposide, doxorubicin, liposomal doxorubicin and bendamustine. According to another embodiment, the corticosteroid is selected from the group consisting of dexamethasone and prednisone. According to another embodiment, the immunomodulating agent is selected from the group consisting of thalidomide, lenalidomide and pomalidomide. According to another embodiment, the proteasome inhibitor is selected from the group consisting of bortezomib, carfilzomib and ixazomib. According to another embodiment, the histone deacetylase (HDAC) inhibitor is panobinostat. According to another embodiment, the monoclonal antibody is selected from the group consisting of daratumumab and elotuzumab. According to another embodiment, the interferon is selected from the group consisting of interferon-α, interferon-β, interferon-γ and interferon-λ.

According to another aspect, the described invention provides an ex vivo method for assessing drug resistance of multiple myeloma cells (MMCs) in a subject suffering from multiple myeloma (MM) comprising: (a) preparing the ex vivo endosteal microenvironment perfused by nutrients and dissolved gas molecules comprising three-dimensional (3D) nodular structures that comprise a 3D-endosteal-like tissue as described above; (b) acquiring bone marrow mononuclear cells (BMMCs) comprising viable multiple myeloma cells (MMCs) from the subject; (c) bringing the BMMCs comprising viable MMCs in contact with the endosteal microenvironment perfused by nutrients and gas molecules to seed the ex vivo endosteal microenvironment with the viable MMCs, the ex vivo endosteal microenvironment perfused by nutrients and gas molecules seeded with viable MMCs forming an ex vivo microenvironment effective to recapitulate spatial and temporal characteristics of a multiple myeloma cancer niche and to maintain viability of the MMCs from the subject; and (d) testing therapeutic efficacy of a therapeutic agent on the viable MMCs maintained by the endosteal microenvironment in the first well adapted to receive a therapeutic agent by 1. contacting the MMCs maintained by the endosteal microenvironment of (d) with a test therapeutic agent; and 2. comparing at least one of viability and level of apoptosis of the MMCs contacted with the test therapeutic agent to an untreated MMC control, wherein the MMCs are resistant to the test therapeutic agent if the test therapeutic agent is not effective to significantly reduce viability of the MMCs or is not effective to increase apoptosis of the MMCs compared to the untreated MMC control.

According to one embodiment of the method, the microbeads are biphasic calcium phosphate (BCP) microbeads, polystyrene (PS) microbeads or a combination thereof. According to another embodiment, the microbeads range in diameter from about 20 μm to about 25 μm. According to another embodiment, the osteocyte cells are primary human osteocytes (ph-OSTs) or murine osteocytes. According to another embodiment, the osteoblast cells (OSBs) are primary human osteoblasts (ph-OSBs). According to another embodiment, the primary human osteoblasts (ph-OSBs) are autologous ph-OSBs. According to another embodiment, the gas molecules are oxygen (O2) molecules. According to another embodiment, the therapeutic agent is selected from the group consisting of a chemotherapeutic agent, a corticosteroid, an immunomodulating agent, a proteasome inhibitor, a histone deacetylase (HDAC) inhibitor, a monoclonal antibody and interferon. According to another embodiment, the chemotherapeutic agent is selected from the group consisting of melphalan, vincristine, cyclophosphamide, etoposide, doxorubicin, liposomal doxorubicin and bendamustine. According to another embodiment, the corticosteroid is selected from the group consisting of dexamethasone and prednisone. According to another embodiment, the immunomodulating agent is selected from the group consisting of thalidomide, lenalidomide and pomalidomide. According to another embodiment, the proteasome inhibitor is selected from the group consisting of bortezomib, carfilzomib and ixazomib. According to another embodiment, the histone deacetylase (HDAC) inhibitor is panobinostat. According to another embodiment, the monoclonal antibody is selected from the group consisting of daratumumab and elotuzumab. According to another embodiment, the interferon is selected from the group consisting of interferon-α, interferon-β, interferon-γ and interferon-λ.

### BRIEF DESCRIPTION OF THE DRAWINGS

For a more complete understanding of the described invention, reference is made to the following detailed description of an exemplary embodiment considered in conjunction with the accompanying drawings.

FIG. 1 depicts (a) established ex vivo replication of patient multiple myeloma cell (PMMC)/osteoblast (OSB) interaction and 3D-networked osteocyte (OST) tissue; (b) adhesion interactions of PMMCs with bone marrow (BM) niches to be studied under 1-20% O2 tension (with endosteal example illustrated); and (c) niche-dependent dormancy, proliferation and drug response of PMMCs to be ascertained under 1-20% O2 tension.

FIG. 2A shows a confocal image of patient multiple myeloma cells (PMMCs) and osteoblasts (OSBs); FIG. 2B shows an image of PMMC/OSB adhesion; and FIG. 2C shows a table of expansion times of PMMC populations after 1 week at 20% O2 or 1% O2 (p<0.01 compared to 1% O2).

FIG. 3A shows response of CD138+ patient multiple myeloma cells (PMMCs) to carfilzomib (CFZ) when cultured with osteoblasts (OSBs). FIG. 3B shows response of CD138+ patient multiple myeloma cells (PMMCs) to melphalan when cultured with OSBs or in fibrin at 1% O2 or 20% O2 tension.

FIGS. 4A-4F show 3D-networked primary human osteocyte (ph-OST) reconstruction at 1% O2. FIG. 4A shows Human bone sample. FIG. 4B shows reconstructed 3D-OST tissue. FIG. 4C and FIG. 4D show hemotoxylin and eosin staining showing 3D-OST tissue with an outer 2D-osteoblast (OSB) layer; dendritic connections (black arrows) between cell bodies (red arrows) and voids left by pulled-out microbeads (dashed circles). FIG. 4E and FIG. 4F show effects of 3D culture, O2 tension and/or parathyroid hormone (PTH) on relative gene expressions; ALPL=alkaline phosphatase liver/bone/kidney lysozyme; DMP-1=dentin matrix acidic phosphoprotein 1; FGF23=fibroblast growth factor 23; SOST=sclerostin; RANKL=receptor activator of nuclear factor kappa-B lingand; OPG=osteoprotegerin.

FIGS. 5A-5F shows data on patient multiple myeloma cell (PMMC)—osteocyte (OST) adhesion. FIG. 5A shows a schematic of culture device. FIG. 5B shows a photograph of culture device. FIG. 5C shows bone marrow mononuclear cells (BMMCs) (blue) adhered to 3D-osteocyte (OST). FIG. 5D shows flow cytometry data of CD138+CD38+ PMMCs adhered to 3D-OST. FIG. 5E shows MML1S cells (green) adhered to 3D-OST. FIG. 5F shows MML1S cells (blue) adhered to 2D-cultured MLO-A5 cells (scale bar=50 μm).

FIG. 6A shows a cross-sectional confocal image of CD138+ patient multiple myeloma cells (PMMCs) dispersed in fibrin prior to culture. FIG. 6B is a table showing the effect of O2 tension on CD138+ PMMC viability and proliferation after 5-day culture in fibrin (p<0.05 to 1% O2).

FIGS. 7A-7C show images of continuous 2D-endothelial cell (EC) layer grown on nanofibrous meshes: FIG. 7A shows an SEM image of nanofibrous mesh; FIG. 7B shows CD31 intercellular marker staining; FIG. 7C shows occluding tight junction staining.

FIG. 8 is a schematic illustration of device fabrication plan for the culture platform illustrated in FIG. 1.

FIG. 9A is an illustration of a 3D-osteocyte (OST)/2D-osteoblast (OSB) tissue surface. FIG. 9B shows simulation results for an O2 tension gradient through 3D-OST/2D-OSB tissue due to metabolic consumption.

FIGS. 10A-10D show an example of high-content screening (HCS) analysis. FIGS. 10A and 10B are images of stained CD138+ patient multiple myeloma cells (PMMCs) (red), bone marrow mononuclear cells (BMMCs) (blue) and osteoblasts (green) from a well of the culture device. FIG. 10C is a chart indicating the number of BMMCs and OSBs in the well of the culture device. FIG. 10D is a chart indicating the number of CD138+ PMMCs in the well of the culture device.

FIG. 11 shows a drawing of an embodiment of the described multiwell plate-based perfusion culture device for co-cultivating recipient subject intestinal epithelial cells and an allogeneic donor's T lymphocytes.

FIGS. 12A and 12B show immunofluorescence analysis of m-CRIECs. Staining was performed using a directly conjugated anti-pan cytokertin antibody on mCRIEC cells. 50 px scale-bar equals 16.1 μm (20×) and 8.05 μm (40×). FIG. 12C shows flow cytometric analysis of extracellular (pan cytokeratin, EpCAM, CD24, and CD44) and intracellular (Lgr5) IEC cell markers.

FIG. 13 shows upregulation of MHC I and II in m-CRIEC. M-CRIEC were cultured in different cell culture media for 72 h in the presence of 20 ng/ml TNF-α and 10 U/ml IFN-γ to induce the upregulation of MHC class 1 (lab) and class II (H2kb) molecules on the surface of m-CRIEC. Maximal MHC II expression was obtained when using cRPMI medium or cRPMI plus a nanofibrous mesh.

FIG. 14 shows morphology of m-CRIECs cultured in CRC medium, cRPMI and cRPMI on nanofibrous mesh for 7 days to evaluate morphology and viability. Loss of cobble stone morphology (black arrow) was observed without nanofibrous mesh.

FIG. 15 shows a killing assay of B10.BR T cells against B6 m-CRIEC. B6 m-CRIEC cultured with nanofibrous mesh and cRPMI were stimulated with 20 ng/ml TNF-α and 10 U/ml IFN-γ for 48-72 h (panel I) and cocultured with MLC-stimulated B10.BR T cells at an E:T ratio of 5:1 (panel II) and 10:1 (panel III). On day 6, m-CRIEC at E:T ratio 10:1 were visibly compromised as determined by trypan blue staining (panel III). Arrows indicate T cells.

FIGS. 16A-16E show a well plate-based perfusion device: FIGS. 16A-16C are schematic illustrations of the device and fabrication; FIG. 16D shows an actual device used for preliminary results; FIG. 16E shows an SEM image of nanofibrous mesh coated onto a PC membrane.

FIG. 17A m-CRIEC from B6 mice were cultured for 7 days in the IEC culture chamber; FIGS. 17B and 17C followed by the introduction of circulating MHC-mismatched B10.BR m-T cells for 5 more days; FIG. 17B SEM; and FIG. 17C fluorescence images showing m-CRIECs and m-T cells. Scale bar: 50 μm.

FIGS. 18A and 18B show flow cytometric analysis of T cell proliferation upon DC stimulation in 2D (FIG. 18A) vs. 3D (FIG. 18B) culture. eGFP B6 T cells were labeled with eFluor 670 and cultured for 4 days with BALB.B DCs to assess proliferation. In comparison to 2D, the total percent (%) of live T cells [undivided cells (U)+proliferating (P)] was greater in 3D, indicating more T cells remained viable in 3D perfusion culture. This enhanced viability was also reflected by the increased R:S ratio (10:1 vs. 2:1). Furthermore, the percentage of proliferating T cells (P) was greater in 3D than in 2D (40% vs. 33%).

FIGS. 19A-19E show circulation of primary murine T cells through primary murine IECs: FIG. 19A shows a schematic illustration of the device configuration and use; FIG. 19B shows an SEM image showing collagen/PCL nanofiber mesh on PC membrane with 10 μm pores; FIG. 19C is a merged fluorescence image showing IEC cytoskeleton (red, ActinRed) and nucleus (blue, DAPI) after day 7 on collagen/PCL nanofiber mesh; FIG. 19D shows effect of IEC presence on T cell viability; and FIG. 19E is a bright field image showing IECs and T cells cultured for 6 h on nanofiber mesh.

### DETAILED DESCRIPTION OF THE INVENTION

### Glossary

Various terms used throughout this specification shall have the definitions set out herein.

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. For example, a mature B cell can be activated by an encounter with an antigen that expresses epitopes that are recognized by its cell surface immunoglobulin (Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC). The soluble product of an activated B lymphocyte is immmunoglobulins (antibodies). The soluble product of an activated T lymphocyte is lymphokines.

The term “administering” as used herein includes in vivo administration, as well as administration directly to tissue ex vivo. Generally, compositions can be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or can be locally administered by means such as, but not limited to, injection, implantation, grafting, topical application, or parenterally.

The term “antigen” and its various grammatical forms refers to any substance that can stimulate the production of antibodies and can combine specifically with them. The term “antigenic determinant” or “epitope” as used herein refers to an antigenic site on a molecule.

An “antiserum” is the liquid phase of blood recovered after clotting has taken place obtained from an immunized mammal, including humans.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone Hi to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon agregation and mitochondrial localization, induces cytochrome c release. Akt is a critical regulator of GSK-3, and phosphorylation and inactivation of GSK-3 may mediate some of the antiapoptotic effects of Akt.

The term “associate” and its various grammatical forms as used herein refers to joining, connecting, or combining to, either directly, indirectly, actively, inactively, inertly, non-inertly, completely or incompletely. The term “in association with” refers to a relationship between two substances that connects, joins or links one substance with another

The term “arrange” as used herein refers to being disposed or placed in a particular kind of order.

The term “Bence Jones protein(s)” as used herein refers to Ig light chain of one type (either κ or λ) that appears in the urine of patients with multiple myeloma.

The term “biomarkers” (or “biosignatures”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “bone” as used herein refers to a hard connective tissue consisting of cells embedded in a matrix of mineralized ground substance and collagen fibers. The fibers are impregnated with a form of calcium phosphate similar to hydroxyapatite as well as with substantial quantities of carbonate, citrate sodium and magnesium. Bone consists of a dense outer layer of compact substance or cortical substance covered by the periosteum and an inner loose, spongy substance; the central portion of a long bone is filled with marrow. The term “bound” or any of its grammatical forms as used herein refers to the capacity to hold onto, attract, interact with or combine with.

The term “bone morphogenic protein (BMP)” as used herein refers to a group of cytokines that are part of the transforming growth factor-ß (TGF-ß) superfamily. BMP ligands bind to a complex of the BMP receptor type II and a BMP receptor type I (Ia or Ib). This leads to the phosphorylation of the type I receptor that subsequently phosphorylates the BMP-specific Smads (Smad1, Smad5, and Smad8), allowing these receptor-associated Smads to form a complex with Smad4 and move into the nucleus where the Smad complex binds a DNA binding protein and acts as a transcriptional enhancer. BMPs have a significant role in bone and cartilage formation in vivo. It has been reported that most BMPs are able to stimulate osteogenesis in mature osteoblasts, while BMP-2, 6, and 9 may play an important role in inducing osteoblast differentiation of mesenchymal stem cells. Cheng, H. et al., J. Bone & Joint Surgery 85: 1544-52 (2003).

The term “cell” is used herein to refer to the structural and functional unit of living organisms and is the smallest unit of an organism classified as living.

The term “cell adhesion” refers to adherence of cells to surfaces or other cells, or to the close adherence (bonding) to adjoining cell surfaces.

The term “cell adhesion molecule” refers to surface ligands, usually glycoproteins, that mediate cell-to-cell adhesion. Their functions include the assembly and interconnection of various vertebrate systems, as well as maintenance of tissue integration, wound healing, morphogenic movements, cellular migrations, and metastasis.

The term “cell-cell interaction” refers to the ways in which living cells communicate, whether by direct contact or by means of chemical signals.

The term “cell culture” as used herein refers to establishment and maintenance of cultures derived from dispersed cells taken from original tissues, primary culture, or from a cell line or cell strain.

The term “cell line” as used herein refers to an immortalized cell, which have undergone transformation and can be passed indefinitely in culture.

The term “cell strain” as used herein refers to cells which can be passed repeatedly but only for a limited number of passages.

The term “cell clones” as used herein refers to individual cells separated from the population and allowed to grow.

The term “primary culture” as used herein refers to cells resulting from the seeding of dissociated tissues, i.e. HUVEC cells. Primary cultures often lose their phenotype and genotypes within several passages.

The term “cell passage” as used herein refers to the splitting (dilution) and subsequent redistribution of a monolayer or cell suspension into culture vessels containing fresh media.

The term “chemokine” as used herein refers to a class of chemotactic cytokines that signal leukocytes to move in a specific direction. The terms “chemotaxis” or “chemotactic” refer to the directed motion of a motile cell or part along a chemical concentration gradient towards environmental conditions it deems attractive and/or away from surroundings it finds repellent.

### Cluster of Differentiation

The cluster of differentiation (CD) system is a protocol used for the identification of cell surface molecules present on white blood cells. CD molecules can act in numerous ways, often acting as receptors or ligands; by which a signal cascade is initiated, altering the behavior of the cell. Some CD proteins do not play a role in cell signaling, but have other functions, such as cell adhesion. Generally, a proposed surface molecule is assigned a CD number once two specific monoclonal antibodies (mAb) are shown to bind to the molecule. If the molecule has not been well-characterized, or has only one mAb, the molecule usually is given the provisional indicator “w.”

The CD system nomenclature commonly used to identify cell markers thus allows cells to be defined based on what molecules are present on their surface. These markers often are used to associate cells with certain immune functions. While using one CD molecule to define populations is uncommon, combining markers has allowed for cell types with very specific definitions within the immune system. There are more than 350 CD molecules identified for humans.

CD molecules are utilized in cell sorting using various methods, including flow cytometry. Cell populations usually are defined using a “+” or a “−” symbol to indicate whether a certain cell fraction expresses or lacks a CD molecule. For example, a “CD34+, CD31−” cell is one that expresses CD34, but not CD31. Table 2 shows commonly used markers employed by skilled artisans to identify and characterize differentiated white blood cell types.

TABLE 2

| Type of Cell | CD Markers |
|---|---|
| Stem cells | CD34+, CD31− |
| All leukocyte groups | CD45+ |
| Granulocyte | CD45+, CD15+ |
| Monocyte | CD45+, CD14+ |
| T lymphocyte | CD45+, CD3+ |
| T helper cell | CD45+, CD3+, CD4+ |
| Cytotoxic T cell | CD45+, CD3+, CD8+ |
| B lymphocyte | CD45+, CD19+ or |
| | CD45+, CD20+ |
| Thrombocyte | CD45+, CD61+ |
| Natural killer cell | CD16+, CD56+, CD3 |

CD molecules used in defining leukocytes are not exclusively markers on the cell surface. Most CD molecules have an important function, although only a small portion of known CD molecules have been characterized. For example, there are over 350 CD for humans identified thus far.

CD3 (TCR complex) is a protein complex composed of four distinct chains. In mammals, the complex contains a CD3γ chain, a CD36 chain, and two CD38 chains, which associate with the T cell receptor (TCR) and the ζ-chain to generate an activation signal in T lymphocytes. Together, the TCR, the ζ-chain and CD3 molecules comprise the TCR complex. The intracellular tails of CD3 molecules contain a conserved motiff known as the immunoreceptor tyrosine-based activation motif (ITAM), which is essential for the signaling capacity of the TCR. Upon phosphorylation of the ITAM, the CD3 chain can bind ZAP70 (zeta associated protein), a kinase involved in the signaling cascade of the T cell.

CD14 is a cell surface protein expressed mainly by macrophages and, to a lesser extent, neutrophil granulocytes. CD14+ cells are monocytes that can differentiate into a host of different cells; for example, differentiation to dendritic cells is promoted by cytokines such as GM-CSF and IL-4. CD14 acts as a co-receptor (along with toll-like receptor (TLR) 4 and lymphocyte antigen 96 (MD-2)) for the detection of bacterial lipopolysaccharide (LPS). CD14 only can bind LPS in the presence of lipopolysaccharide binding protein (LBP).

CD15 (3-fucosyl-N-acetyl-lactosamine; stage specific embryonic antigen 1 (SSEA-1)) is a carbohydrate adhesion molecule that can be expressed on glycoproteins, glycolipids and proteoglycans. CD15 commonly is found on neutrophils and mediates phagocytosis and chemotaxis.

CD16 is an Fc receptor (FcγRIIIa and FcγRIIIb) found on the surface of natural killer cells, neutrophil polymorphonuclear leukocytes, monocytes and macrophages. Fc receptors bind to the Fc portion of IgG antibodies.

CD19 is a human protein expressed on follicular dendritic cells and B cells. This cell surface molecule assembles with the antigen receptor of B lymphocytes in order to decrease the threshold for antigen receptor-dependent stimulation. It generally is believed that, upon activation, the cytoplasmic tail of CD19 becomes phosphorylated, which allows binding by Src-family kinases and recruitment of phosphoinositide 3 (PI-3) kinases.

CD20 is a non-glycosylated phosphoprotein expressed on the surface of all mature B-cells. Studies suggest that CD20 plays a role in the development and differentiation of B-cells into plasma cells. CD20 is encoded by a member of the membrane-spanning 4A gene family (MS4A). Members of this protein family are characterized by common structural features and display unique expression patterns among hematopoietic cells and nonlymphoid tissues.

CD31 (platelet/endothelial cell adhesion molecule; PECAM1) normally is found on endothelial cells, platelets, macrophages and Kupffer cells, granulocytes, T cells, natural killer cells, lymphocytes, megakaryocytes, osteoclasts and neutrophils. CD31 has a key role in tissue regeneration and in safely removing neutrophils from the body. Upon contact, the CD31 molecules of macrophages and neutrophils are used to communicate the health status of the neutrophil to the macrophage.

CD34 is a monomeric cell surface glycoprotein normally found on hematopoietic cells, endothelial progenitor cells, endothelial cells of blood vessels, and mast cells. The CD34 protein is a member of a family of single-pass transmembrane sialomucin proteins and functions as a cell-cell adhesion factor. Studies suggest that CD34 also may mediate the attachment of stem cells to bone marrow extracellular matrix or directly to stromal cells.

CD45 (protein tyrosine phosphatase, receptor type, C; PTPRC) cell surface molecule is expressed specifically in hematopoietic cells. CD45 is a protein tyrosine phosphatase (PTP) with an extracellular domain, a single transmembrane segment, and two tandem intracytoplasmic catalytic domains, and thus belongs to receptor type PTP. Studies suggest it is an essential regulator of T-cell and B-cell antigen receptor signaling that functions by direct interaction with components of the antigen receptor complexes, or by activating various Src family kinases required for antigent receptor signaling. CD45 also suppresses JAK kinases, and thus functions as a regulator of cytokine receptor signaling. The CD45 family consists of multiple members that are all products of a single complex gene. Various known isoforms of CD45 include: CD45RA, CD45RB, CD45RC, CD45RAB, CD45RAC, CD45RBC, CD45RO, and CD45R (ABC). Different isoforms may be found on different cells. For example, CD45RA is found on naïve T cells and CD45RO is found on memory T cells.

CD56 (neural cell adhesion molecule, NCAM) is a homophilic binding glycoprotein expressed on the surface of neurons, glia, skeletal muscle and natural killer cells. It generally is believed that NCAM has a role in cell-cell adhesion, neurite outgrowth, and synaptic plasticity. There are three known main isoforms of NCAM, each varying only in their cytoplasmic domains: NCAM-120 kDA (glycosylphopharidylinositol (GPI) anchored); NCAM-140 kDa (short cytoplasmic domain); and NCAM (long cytoplasmic domain). The different domains of NCAM have different roles, with the Ig domains being involved in homophilic binding to NCAM, and the fibronection type III (FNIII) domains being involved in signaling leading to neurite outgrowth.

CD66b ((CGM1); CD67, CGM6, NCA-95) is a glycosylphosphatidylinositol (GPI)-linked protein that is a member of the immunoglobulin superfamily and carcinoembryonic antigen (CEA)-like subfamily. CD66b, expressed on granulocytes, generally is believed to be involved in regulating adhesion and activation of human eosinophils.

Human leukocyte antigen (HLA)-DR is a major histocompatibility complex (MHC) class II cell surface receptor. HLA-DR commonly is found on antigen-presenting cells such as macrophages, B-cells, and dendritic cells. This cell surface molecule is a αβ heterodimer with each subunit containing 2 extracellular domains: a membrane spanning domain and a cytoplasmic tail. Both the α and β chains are anchored in the membrane. The complex of HLA-DR and its ligand (a peptide of at least 9 amino acids) constitutes a ligand for the TCR.

Integrins are receptors that mediate attachment between a cell and the tissues surrounding it and are involved in cell-cell and cell-matrix interactions. In mammals, 18 α and 8 β subunits have been characterized. Both α and β subunits contain two separate tails, both of which penetrate the plasma membrane and possess small cytoplasmic domains.

Integrin αM (ITGAM; CD11b; macrophage-1 antigen (Mac-1); complement receptor 3 (CR3)) is a protein subunit of the heterodimeric integrin αMβ2 molecule. The second chain of αMβ2 is the common integrin β2 subunit (CD18). αMβ2 is expressed on the surface of many leukocytes including monocytes, granulocytes, macrophages and natural killer cells. It generally is believed that of αMβ2 mediates inflammation by regulating leukocyte adhesion and migration. Further, of αMβ2 is thought to have a role in phagocytosis, cell-mediated cytotoxicity, chemotaxis and cellular activation, as well as being involved in the complement system due to its capacity to bind inactivated complement component 3b (iC3b). The ITGAM subunit of integrin of αMβ2 is involved directly in causing the adhesion and spreading of cells, but cannot mediate cellular migration without the presence of the β2 (CD18) subunit.

CD61 (integrin β3; platelet glycoprotein IIIa; ITGB3) is a cell surface protein composed of an α-chain and a β-chain. A given chain may combine with multiple partners resulting in different integrins. CD61 is found along with the α IIb chain in platelets and is known to participate in cell adhesion and cell-surface mediated signaling.

CD63 (LAMP-3; ME491; MLA1; OMA81H) is a cell surface glycoprotein of the transmembrane 4 superfamily (tetraspanin family). Many of these cell surface receptors have four hydrophobic domains and mediate signal transduction events that play a role in the regulation of cell development, activation, growth and motility. CD63 forms complexes with integrins and may function as a blood platelet activation marker. It generally is believed that the sensitivity and specificity of measuring the upregulation of CD63 alone, or as part of a combination, is not specific enough to serve as a diagnostic marker for the diagnosis of IgE mediated allergy.

CD123 is the 70 kD transmembrane a chain of the cytokine interleukin-3 (IL-3) receptor. Alone, CD123 binds IL-3 with low affinity; when CD123 associates with CDw131 (common β chain), it binds IL-3 with high affinity. CD123 does not transduce intracellular signals upon binding IL-3 and requires the β chain for this function. CD123 is expressed by myeloid precursors, macrophages, dendritic cells, mast cells, basophils, megakaryocytes, and some B cells CD123 induces tyrosine phosphorylation within the cell and promotes proliferation and differentiation within the hematopoietic cell lines.

CD203c (ectonucleotide pyrophosphatase/phosphodiesterase 3; ENPP3) is an ectoenzyme constitutively and specifically expressed on the cell surface and within intracellular compartments of basophils, mast cells, and precursors of these cells. CD203c detection by flow cytometry has been used to specifically identify basophils within a mixed leukocyte suspension, since its expression is unique to basophils among the cells circulating in blood. The expression of CD203c is both rapidly and markedly upregulated following IgE-dependent activation. However, as with CD63, it is generally believed that the sensitivity and specificity of measuring the upregulation of CD203c alone, or as part of a combination, is not specific enough to serve as a diagnostic marker for the diagnosis of IgE mediated allergy. Further, the exact role of CD203c in basophil biology is unknown.

CD294 (G protein-coupled receptor 44; GPR44; CRTh2; DP2) is an integral membrane protein. This chemoattractant receptor homologous molecule is expressed on T helper type-2 cells. The transmembrane domains of these proteins mediate signals to the interior of the cell by activation of heterotrimeric G proteins that in turn activate various effector proteins that ultimately result a physiologic response.

The term “clone” as used herein refers to a population of cells formed by repeated division from a common cell.

The term “compatible” as used herein means that the components of a composition are capable of being combined with each other in a manner such that there is no interaction that would substantially reduce the efficacy of the composition under ordinary use conditions.

The term “Complement” as used herein refers to a system of plasma proteins that interact with pathogens to mark them for destruction by phagocytes. Complement proteins can be activated directly by pathogens or indirectly by pathogen-bound antibody, leading to a cascade of reactions that occurs on the surface of pathogens and generates active components with various effector functions.

The term “composition” as used herein refers to an aggregate material formed of two or more substances.

The transitional term “comprising”, which is synonymous with “including,” “containing,” or “characterized by,” is inclusive or open-ended and does not exclude additional, unrecited elements or method steps.

The term “concentration” as used herein refers to the amount of a substance in a given volume.

The term “concurrent” as used herein refers to occurring, or to operating, before, during or after an event, episode or time period.

The term “component” as used herein refers to a constituent part, element or ingredient.

The term “condition”, as used herein, refers to a variety of health states and is meant to include disorders or diseases caused by any underlying mechanism or injury.

The term “connected” as used herein refers to being joined, linked, or fastened together in close association.

The term “contact” as used herein refers to the state or condition of touching or being in immediate proximity.

The term “culture” as used herein refers to the cultivation of cells in or on a controlled or defined medium. The terms “culture-expanded” or “expanded” are used interchangeably to refer to an increase in the number of cells by cultivation of the cells in or on a controlled or defined medium.

The term “cytokine” as used herein refers to small soluble protein substances secreted by cells, which have a variety of effects on other cells. Cytokines mediate many important physiological functions, including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins including interleukin 2 (IL-2), as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “cytometry” as used herein, refers to a process in which physical and/or chemical characteristics of single cells, or by extension, of other biological or nonbiological particles in roughly the same size or stage, are measured. In flow cytometry, the measurements are made as the cells or particles pass through the measuring apparatus (a flow cytometer) in a fluid stream. A cell sorter, or flow sorter, is a flow cytometer that uses electrical and/or mechanical means to divert and to collect cells (or other small

The term “dendritic cells” (DCs) as used herein, refers to professional APCs capable of presenting both MHC-I and MHC-II antigens.

The phrase “density-dependent inhibition of growth” as used herein refers to reduced response of cells upon reaching a threshold density. These cells recognize the boundaries of neighbor cells upon confluence and respond, depending on growth patterns, by forming a monolayer. Usually these cells transit through the cell cycle at reduce rate (grow slower).

The term “detectable response” refers to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “derivative” as used herein means a compound that may be produced from another compound of similar structure in one or more steps. A “derivative” or “derivatives” of a peptide or a compound retains at least a degree of the desired function of the peptide or compound. Accordingly, an alternate term for “derivative” may be “functional derivative.”

The term “derived from” as used herein is used to refer to originating, sourced, or coming from.

The term “differential label” as used herein, generally refers to a stain, dye, marker, antibody or antibody-dye combination, or intrinsically fluorescent cell-associated molecule, used to characterize or contrast components, small molecules, macromolecules, e.g., proteins, and other structures of a single cell or organism. The term “dye” (also referred to as “fluorochrome” or “fluorophore”) as used herein refers to a component of a molecule which causes the molecule to be fluorescent. The component is a functional group in the molecule that absorbs energy of a specific wavelength and re-emits energy at a different (but equally specific) wavelength. The amount and wavelength of the emitted energy depend on both the dye and the chemical environment of the dye. Many dyes are known, including, but not limited to, FITC, R-phycoerythrin (PE), PE-Texas Red Tandem, PE-Cy5 Tandem, propidium iodem, EGFP, EYGP, ECF, DsRed, allophycocyanin (APC), PerCp, SYTOX Green, courmarin, Alexa Fluors (350, 430, 488, 532, 546, 555, 568, 594, 633, 647, 660, 680, 700, 750), Cy2, Cy3, Cy3.5, Cy5, Cy5.5, Cy7, Hoechst 33342, DAPI, Hoechst 33258, SYTOX Blue, chromomycin A3, mithramycin, YOYO-1, SYTOX Orange, ethidium bromide, 7-AAD, acridine orange, TOTO-1, TO-PRO-1, thiazole orange, TOTO-3, TO-PRO-3, thiazole orange, propidium iodide (PI), LDS 751, Indo-1, Fluo-3, DCFH, DHR, SNARF, Y66F, Y66H, EBFP, GFPuv, ECFP, GFP, AmCyanl, Y77W, S65A, S65C, S65L, S65T, ZsGreenl, ZsYellowl, DsRed2, DsRed monomer, AsRed2, mRFP1, HcRedl, monochlorobimane, calcein, the DyLight Fluors, cyanine, hydroxycoumarin, aminocoumarin, methoxycoumarin, Cascade Blue, Lucifer Yellow, NBD, PE-Cy5 conjugates, PE-Cy7 conjugates, APC-Cy7 conjugates, Red 613, fluorescein, FluorX, BODIDY-FL, TRITC, X-rhodamine, Lissamine Rhodamine B, Texas Red, TruRed, and derivatives thereof.

The term “differentiation” as used herein refers to a property of cells to exhibit tissue-specific differentiated properties in culture.

The term “dissolved gas molecules” as used herein refers to molecules (e.g., O2, CO2, etc.) dissolved in cell culture medium.

The term “disease” or “disorder,” as used herein, refers to an impairment of health or a condition of abnormal functioning.

The term “drug” as used herein refers to a therapeutic agent or any substance used in the prevention, diagnosis, alleviation, treatment, or cure of disease.

The term “dynamic” as used herein refers to changing conditions to which an agent must adapt.

The term “endosteal” as used herein refers to a connective tissue that lines the surface of bony tissue that forms the medullary cavity of long bones.

The term “extracellular matrix” as used herein refers to a construct in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fibronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached one or more glycosaminoglycans.

### Flow Cytometry

Flow cytometry is a technique for counting, examining, and sorting microscopic particles suspended in a stream of fluid. It allows simultaneous multi-parametric analysis of the physical and/or chemical characteristics of single cells flowing through an optical and/or electronic detection apparatus.

Flow cytometry utilizes a beam of light (usually laser light) of a single wavelength that is directed onto a hydro-dynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals found in the particle or attached to the particle may be excited into emitting light at a lower frequency than the light source. This combination of scattered and fluorescent light is picked up by the detectors, and by analyzing fluctuations in brightness at each detector (usually one for each fluorescent emission peak) it then is possible to derive various types of information about the physical and chemical structure of each individual particle. FSC correlates with the cell volume and SSC depends on the inner complexity of the particle (i.e. shape of the nucleus, the amount and type of cytoplasmic granules or the membrane roughness).

FACS

The term “fluorescence-activated cell sorting” (also referred to as “FACS”), as used herein, refers to a method for sorting a heterogeneous mixture of biological cells into one or more containers, one cell at a time, based upon the specific light scattering and fluorescent characteristics of each cell.

Fluorescence-activated cell sorting (FACS) is a specialized type of flow cytometry. It provides a method for sorting a heterogeneous mixture of biological cells into two or more containers, one cell at a time, based upon the specific light scattering and fluorescent characteristics of each cell. It provides fast, objective and quantitative recording of fluorescent signals from individual cells as well as physical separation of cells of particular interest.

Utilizing FACS, a cell suspension is entrained in the center of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell being in a droplet. Before the stream breaks into droplets the flow passes through a fluorescence measuring station where the fluorescent character of interest of each cell is measured. An electrical charging ring or plane is placed just at the point where the stream breaks into droplets. A charge is placed on the ring based on the prior light scatter and fluorescence intensity measurements, and the opposite charge is trapped on the droplet as it breaks from the stream. The charged droplets then fall through an electrostatic deflection system that diverts droplets into containers based upon their charge. In some systems the charge is applied directly to the stream while a nearby plane or ring is held at ground potential and the droplet breaking off retains charge of the same sign as the stream. The stream is then returned to neutral after the droplet breaks off.

The term “growth” as used herein refers to a process of becoming larger, longer or more numerous, or an increase in size, number, or volume.

The term “growth factor” as used herein refers to signal molecules involved in the control of cell growth and differentiation and cell survival.

The term “hybridoma cell” as used herein refers to an immortalized hybrid cell resulting from the in vitro fusion of an antibody-secreting B cell with a myeloma cell. For example, monoclonal antibodies (mAbs) can be generated by fusing mouse spleen cells from an immunized donor with a mouse myeloma cell line to yield established mouse hybridoma clones that grow in selective media.

The term “immunoglobulin (Ig)” as used herein refers to one of a class of structurally related proteins, each consisting of two pairs of polypeptide chains, one pair of identical light (L) (low molecular weight) chains (κ or λ), and one pair of identical heavy (H) chains (γ, α, μ, δ and ε), usually all four linked together by disulfide bonds. On the basis of the structural and antigenic properties of the H chains, Igs are classified (in order of relative amounts present in normal human serum) as IgG, IgA, IgM, IgD, and IgE. Each class of H chain can associate with either κ or λ L chains. There are four subclasses of IgG immunoglobulins (IgG1, IgG2, IgG3, IgG4) having γ1, γ2, γ3, and γ4 heavy chains respectively. In its secreted form, IgM is a pentamer composed of five four-chain units, giving it a total of 10 antigen binding sites. Each pentamer contains one copy of a J chain, which is covalently inserted between two adjacent tail regions.

The term Ig refers not only to antibodies, but also to pathological proteins classified as myeloma proteins, which appear in multiple myeloma along with Bence Jones proteins, myeloma globulins, and Ig fragments.

Antibodies are serum proteins the molecules of which possess small areas of their surface that are complementary to small chemical groupings on their targets. Both light and heavy chains usually cooperate to form the antigen binding surface. These complementary regions (referred to as the antibody combining sites or antigen binding sites) of which there are at least two per antibody molecule, and in some types of antibody molecules ten, eight, or in some species as many as 12, may react with their corresponding complementary region on the antigen (the antigenic determinant or epitope) to link several molecules of multivalent antigen together to form a lattice.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions.

All five immunoglobulin classes differ from other serum proteins in that they normally show a broad range of electrophoretic mobility and are not homogeneous. This heterogeneity—that individual IgG molecules, for example, differ from one another in net charge—is an intrinsic property of the immunoglobulins, and accounts for the libraries of antibodies each individual possesses.

The term “immunoglobulin fragment” (“Ig fragment”) refers to a partial immunoglobulin molecule.

The term “in vitro immunization” is used herein to refer to primary activation of antigen-specific B cells in culture.

The term “inhibit” and its various grammatical forms, including, but not limited to, “inhibiting” or “inhibition”, are used herein to refer to reducing the amount or rate of a process, to stopping the process entirely, or to decreasing, limiting, or blocking the action or function thereof. Inhibition can include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a second molecule that binds to a first molecule thereby decreasing the first molecule's activity. Enzyme inhibitors are molecules that bind to enzymes thereby decreasing enzyme activity. The binding of an inhibitor can stop a substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “injury,” as used herein, refers to damage or harm to a structure or function of the body caused by an outside agent or force, which can be physical or chemical.

The term “immunomodulatory cell(s)” as used herein refer(s) to cell(s) that are capable of augmenting or diminishing immune responses by expressing chemokines, cytokines and other mediators of immune responses.

The term “inflammatory cytokines” or “inflammatory mediators” as used herein refers to the molecular mediators of the inflammatory process, which may modulate being either pro- or anti-inflammatory in their effect. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, pro-inflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12).

The term “interacted with” as used herein refers to a kind of action that occurs as two or more objects have an effect upon one another.

The term “interleukin (IL)” as used herein refers to a cytokine secreted by, and acting on, leukocytes. Interleukins regulate cell growth, differentiation, and motility, and stimulates immune responses, such as inflammation. Examples of interleukins include interleukin-1 (IL-1), interleukin 2 (IL-2), interleukin-1β (IL-1β), interleukin-6 (IL-6), interleukin-8 (IL-8), and interleukin-12 (IL-12).

The term “isolated” is used herein to refer to material, such as, but not limited to, a cell, nucleic acid, peptide, polypeptide, or protein, which is: (1) substantially or essentially free from components that normally accompany or interact with it as found in its naturally occurring environment.

The term “Kaplan Meier plot” or “Kaplan Meier survival curve” as used herein refers to a plot of probability of clinical study patients surviving in a given length of time while considering time in many small intervals. The Kaplan Meier plot assumes that: (i) at any time patients who are censored (i.e., lost) have the same survival prospects as patients who continue to be followed; (ii) the survival probabilities are the same for patients recruited early and late in the study; and (iii) the event (e.g., death) happens at the time specified. Probabilities of occurrence of event are computed at a certain point of time with successive probabilities multiplied by any earlier computed probabilities to get a final estimate. The survival probability at any particular time is calculated as the number of patients surviving divided by the number of patients at risk. Patients who have died, dropped out, or have been censored from the study are not counted as at risk.

The terms “label” or “labeled” as used herein refers to incorporation of a detectable marker or molecule.

The term “marker’ as used herein refers to a receptor, or a combination of receptors, found on the surface of a cell. These markers allow a cell type to be distinguishable from other kinds of cells. Specialized protein receptors (markers) that have the capability of selectively binding or adhering to other signaling molecules coat the surface of every cell in the body. Cells use these receptors and the molecules that bind to them as a way of communicating with other cells and to carry out their proper function in the body.

The term “matrix” as sued herein refers to a three dimensional network of fibers that contains voids (or “pores”) where the woven fibers intersect. The structural parameters of the pores, including the pore size, porosity, pore interconnectivity/tortuosity and surface area, affect how fluid, solutes and cells move in and out of the matrix.

The term “microfluidics” refers to a set of technologies that control the flow of minute amounts of liquids or dissolved gas molecules, typically measured in nano- and pico-liters in a miniaturized system. The microchips require only a small amount of sample and reagent for each process, and microscale reactions occur much faster because of the physics of small fluid volumes.

The term “modulate” as used herein means to regulate, alter, adapt, or adjust to a certain measure or proportion.

The term “monoclonal” as used herein refers to resulting from the proliferation of a single clone.

The term “monoclonal Ig” as used herein refers to a homogeneous immunoglobulin resulting from the proliferation of a single clone of plasma cells and which, during electrophoresis of serum, appears as a narrow band or “spike”. It is characterized by H chains of a single class and subclass, and light chains of a single type.

The term “monolayer” as used herein refers to a layer of cells one cell thick, grown in a culture.

As used herein, the terms “osteoprogenitor cells,” “mesenchymal cells,” “mesenchymal stem cells (MSC),” or “marrow stromal cells” are used interchangeably to refer to multipotent stem cells that differentiate from CFU-F cells capable of differentiating along several lineage pathways into osteoblasts, chondrocytes, myocytes and adipocytes. When referring to bone or cartilage, MSCs commonly are known as osteochondrogenic, osteogenic, chondrogenic, or osteoprogenitor cells, since a single MSC has shown the ability to differentiate into chondrocytes or osteoblasts, depending on the medium.

The term “osteoblasts” as used herein refers to cells that arise when osteoprogenitor cells or mesenchymal cells, which are located near all bony surfaces and within the bone marrow, differentiate under the influence of growth factors. Osteoblasts, which are responsible for bone matrix synthesis, secrete a collagen rich ground substance essential for later mineralization of hydroxyapatite and other crystals. The collagen strands to form osteoids: spiral fibers of bone matrix. Osteoblasts cause calcium salts and phosphorus to precipitate from the blood, which bond with the newly formed osteoid to mineralize the bone tissue. Once osteoblasts become trapped in the matrix they secrete, they become osteocytes. From least to terminally differentiated, the osteocyte lineage is (i) Colony-forming unit-fibroblast (CFU-F); (ii) mesenchymal stem cell/marrow stromal cell (MSC); (3) osteoblast; (4) osteocyte.

The term “osteogenesis” refers to the formation of new bone from bone forming or osteocompetent cells.

The term “osteocalcin” as used herein refers to a protein constituent of bone; circulating levels are used as a marker of increased bone turnover.

The term “osteoclast” as used herein refers to the large multinucleate cells associated with areas of bone resorption bone resorption (breakdown).

The term “osteogenic factors” refers to the plethora of mediators associated with bone development and repair, including, but not limited to bone morphogenic proteins (BMPs), vascular endothelial growth factor (VEGF), basic fibroblast growth factor (bFGF), transforming growth factor beta (TGFβ), and platelet-derived growth factor (PDGF).

The term “overall survival” (OS) as used herein refers to the length of time from either the date of diagnosis or the start of treatment for a disease that subjects diagnosed with the disease are still alive.

The term “perfusion” as used herein refers to the process of nutritive delivery of arterial blood to a capillary bed in biological tissue. Perfusion (“F”) can be calculated with the formula F=((PA−Pv)/R) wherein PA is mean arterial pressure, Pv is mean venous pressure, and R is vascular resistance. Tissue perfusion can be measured in vivo, by, for example, but not limited to, magnetic resonance imaging (MRI) techniques. Such techniques include using an injected contrast agent and arterial spin labeling (ASL) (wherein arterial blood is magnetically tagged before it enters into the tissue of interest and the amount of labeling is measured and compared to a control recording). Tissue perfusion can be measured in vitro, by, for example, but not limited to, tissue oxygen saturation (StO2) using techniques including, but not limited to, hyperspectral imaging (HSI).

The term “polymer” as used herein refers to a macromolecule formed by the chemical union of five or more identical combining units (monomers). Exemplary polymers by type include, without limitation, inorganic polymers (e.g., siloxane, sulfur chains, black phosphorus, boron-nitrogen, aluminosilicate, borosilicate, or boro-aluminosilicate, glass ceramics, ceramics, and semiconductor or crystalline materials (e.g. silicones); Organic polymers, including natural organic polymers e.g., polysaccharides, such as starch, cellulose, pectin, seaweed gums (agar, etc), vegetable gums (Arabic, etc.); polypeptides (e.g., albumin, globulin); and hydrocarbons, e.g., polyisoprene; synthetic polymers, including thermoplastic polymers, such as polyvinyl chloride, polyethylene (linear), polystyrene, polypropylene, fluorocarbon resins, polyurethane, and acrylate resins, and thermosetting synthetic polymers, such as elastomers, polyethylene (cross-linked), penolics, and polyesters; and semisynthetic organic polymers, such as cellulosics (e.g., methylcellulose, cellulose acetate) and modified starches. Further examples of polymers include, without limitation, hydrophilic polyethylene, polystyrenes, polypropylenes, acrylates, methacrylates, polycarbonates, polysulfones, polyesterketones, poly- or cyclic olefins, polychlorotrifluoroethylene, and polyethylene therephthalate.

The term “progression free survival” or “PFS” as used herein refers to length of time during and after the treatment of a disease, such as cancer, that a patient lives with the disease but it does not get worse. In a clinical trial, measuring the progression free survival is one way to determine how well a new treatment works.

The terms “proliferation” and “propagation” are used interchangeably herein to refer to expansion of a population of cells by the continuous division of single cells into identical daughter cells.

The term “reduce” or “reducing” as used herein refers to the limiting of an occurrence of a disease, disorder or condition in individuals at risk of developing the disorder.

The term “relapse” as used herein refers to the return of a disease or the signs and symptoms of a disease after a period of improvement.

The term “relapse-free survival (RFS)” as used herein refers to the length of time after primary treatment for a cancer during which the patient survives without any signs or symptoms of that cancer. Also called disease-free survival (DFS).

The term “stimulate” in any of its grammatical forms as used herein refers to inducing activation or increasing activity.

As used herein, the terms “subject” or “individual” or “patient” are used interchangeably to refer to a member of an animal species of mammalian origin, including humans. The term “a subject in need thereof” is used to refer to a subject who presents with presents with diagnostic markers and symptoms associated with multiple myeloma and either (i) will be in need of treatment, (ii) is receiving treatment; or (iii) has received treatment, unless the context and usage of the phrase indicates otherwise.

The term “suspension culture” as used herein refers to cells which do not require attachment to a substratum to grow, i.e. they are anchorage independent. Cell cultures derived from blood are typically grown in suspension. Cells can grow as single cells or clumps. To subculture the cultures which grow as single cells they can be diluted. However, the cultures containing clumps need to have the clumps disassociated prior to subculturing of the culture.

The term “symptom” as used herein refers to a phenomenon that arises from and accompanies a particular disease or disorder and serves as an indication of it.

The term “syndrome,” as used herein, refers to a pattern of symptoms indicative of some disease or condition.

The term “target” as used herein refers to a biological entity, such as, for example, but not limited to, a protein, cell, organ, or nucleic acid, whose activity can be modified by an external stimulus. Depending upon the nature of the stimulus, there may be no direct change in the target, or a conformational change in the target may be induced.

The term “therapeutic agent” as used herein refers to a drug, molecule, nucleic acid, protein, metabolite, composition or other substance that provides a therapeutic effect. The term “active” as used herein refers to the ingredient, component or constituent of the compositions of the described invention responsible for the intended therapeutic effect. The terms “therapeutic agent” and “active agent” are used interchangeably herein. The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The terms “therapeutic amount”, “therapeutically effective amount”, an “amount effective”, or “pharmaceutically effective amount” of an active agent is used interchangeably to refer to an amount that is sufficient to provide the intended benefit of treatment.

The term “therapeutic effect” as used herein refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The term “therapeutic window” refers to a concentration range that provides therapeutic efficacy without unacceptable toxicity. Following administration of a dose of a drug, its effects usually show a characteristic temporal pattern. A lag period is present before the drug concentration exceeds the minimum effective concentration (“MEC”) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. Accordingly, the duration of a drug's action is determined by the time period over which concentrations exceed the MEC. The therapeutic goal is to obtain and maintain concentrations within the therapeutic window for the desired response with a minimum of toxicity. Drug response below the MEC for the desired effect will be subtherapeutic, whereas for an adverse effect, the probability of toxicity will increase above the MEC. Increasing or decreasing drug dosage shifts the response curve up or down the intensity scale and is used to modulate the drug's effect. Increasing the dose also prolongs a drug's duration of action but at the risk of increasing the likelihood of adverse effects. Accordingly, unless the drug is nontoxic, increasing the dose is not a useful strategy for extending a drug's duration of action.

Instead, another dose of drug should be given to maintain concentrations within the therapeutic window. In general, the lower limit of the therapeutic range of a drug appears to be approximately equal to the drug concentration that produces about half of the greatest possible therapeutic effect, and the upper limit of the therapeutic range is such that no more than about 5% to about 10% of patients will experience a toxic effect. These figures can be highly variable, and some patients may benefit greatly from drug concentrations that exceed the therapeutic range, while others may suffer significant toxicity at much lower values. The therapeutic goal is to maintain steady-state drug levels within the therapeutic window. For most drugs, the actual concentrations associated with this desired range are not and need not be known, and it is sufficient to understand that efficacy and toxicity are generally concentration-dependent, and how drug dosage and frequency of administration affect the drug level. For a small number of drugs where there is a small (two- to three-fold) difference between concentrations resulting in efficacy and toxicity, a plasma-concentration range associated with effective therapy has been defined.

In this case, a target level strategy is reasonable, wherein a desired target steady-state concentration of the drug (usually in plasma) associated with efficacy and minimal toxicity is chosen, and a dosage is computed that is expected to achieve this value. Drug concentrations subsequently are measured and dosage is adjusted if necessary to approximate the target more closely.

In most clinical situations, drugs are administered in a series of repetitive doses or as a continuous infusion to maintain a steady-state concentration of drug associated with the therapeutic window. To maintain the chosen steady-state or target concentration (“maintenance dose”), the rate of drug administration is adjusted such that the rate of input equals the rate of loss. If the clinician chooses the desired concentration of drug in plasma and knows the clearance and bioavailability for that drug in a particular patient, the appropriate dose and dosing interval can be calculated.

The term “two-dimensional tissue construct” as used herein refers to a collection of cells and the intercellular substances surrounding them in a geometric configuration having length and width.

The term “three-dimensional tissue construct” as used herein refers to a tissue like collection of cells and the intercellular substances surrounding them in a geometric configuration having length, width, and depth.

The term “transplantation” as used herein, refers to removal and transfer of cells, a tissue or an organ from one part or individual to another.

As used herein the term “treating” includes abrogating, substantially inhibiting, slowing or reversing the progression of a condition, substantially ameliorating clinical symptoms of a condition, or substantially preventing the appearance of clinical symptoms of a condition. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “tumor necrosis factor” (TNF) as used herein refers to a cytokine made by white blood cells in response to an antigen or infection, which induce necrosis (death) of tumor cells and possesses a wide range of pro-inflammatory actions. Tumor necrosis factor also is a multifunctional cytokine with effects on lipid metabolism, coagulation, insulin resistance, and the function of endothelial cells lining blood vessels.

The terms “VEGF-1” or “vascular endothelial growth factor-1” are used interchangeably herein to refer to a cytokine that mediates numerous functions of endothelial cells including proliferation, migration, invasion, survival, and permeability. VEGF is critical for angiogenesis.

### I. An Ex-Vivo Model of a 3D Cellular Network Found in Native Bones Created by Biomimetic Assembly of Osteocytes and Microbeads in a Microfluidic Perfusion Culture Device

According to some embodiments, the described invention provides an ex vivo model of a three dimensional (3D) cellular network found in native bones via biomimetic assembly of osteocytes and microbeads in a microfluidic perfusion culture device comprising

(a) preparing an in vitro multiwell plate-based perfusion culture device, comprising, from top to bottom:

1. a bottomless multi-well plate comprising a plurality of bottomless wells;

2. a first micropatterned polymer layer attached to a bottom surface of the bottomless multi-well plate to form a plurality of adjacent wells, one or more of each pair of adjacent wells comprising a transparent polymer membrane placed within the one or more of each pair of adjacent wells;

3. a second micropatterned polymer layer comprising two or more holes that correspond to two or more adjacent wells, the second micropatterned polymer layer being attached to a bottom surface of the first micropatterned polymer layer, such that each hole of the second micropatterned polymer layer is aligned with the two or more adjacent wells in the first micropatterned polymer layer, one or more of each pair of adjacent wells comprising the transparent polymer membrane;

4. a microfluidic channel formed between the two adjacent wells that allows internal fluidic communication between the two adjacent wells;

5. one or more removable polymer plugs, each located at a top surface of each of the plurality of wells, and one or more tubes, each connected to the one or more polymer plugs;

6. a pump connected to a reservoir that removably connects to the tubes;

7. a transparent, optical grade glass layer attached to the bottom surface of the second micropatterned polymer layer that forms a bottom surface for the plurality of wells and that seals the multi-well plate perfusion culture device;

wherein

(i) one or more of the two adjacent wells is a cell culture chamber comprising a first well region including a first well and a second well region including a second well;

(ii) the microfluidic channel connects the first well region and the second well region with one another;

(iii) the first well is adapted to receive a therapeutic agent, the second well is adapted to receive a biological sample of cells; and

(iv) liquids, nutrients and dissolved gas molecules flow through the channel;

(b) constructing an ex vivo endosteal microenvironment perfused by nutrients and dissolved gas molecules by;

1. seeding a surface of the culture chamber of the device of (a) with
(i) microbeads;
(ii) osteocyte cells (OSTs); and
(iii) osteoblast cells (OSBs),

2. culturing the cells with a culture medium through the microfluidic channel for a time effective for the cells to form three-dimensional (3D) nodular structures that comprise a 3D-endosteal-like tissue.

According to some embodiments, the microbeads are biphasic calcium phosphate (BCP) microbeads, polystyrene (PS) microbeads or a combination thereof. According to some embodiments, the microbeads range in diameter from about 20 μm to about 25 μm. According to some embodiments, the microbeads are about 20 μm in diameter. According to some embodiments, the microbeads are about 21 μm in diameter. According to some embodiments, the microbeads are about 22 μm in diameter. According to some embodiments, the microbeads are about 23 μm in diameter. According to some embodiments, the microbeads are about 24 μm in diameter. According to some embodiments, the microbeads are about 25 μm in diameter.

### II. An Ex Vivo Model of an Ex Vivo Dynamic Multiple Myeloma (MM) Cancer Niche Comprising a Multiwell Plate-Based Perfusion Culture Device

According to one aspect, an ex vivo model of an ex vivo dynamic multiple myeloma (MM) cancer niche comprises

(a) A multiwell plate-based perfusion culture device, comprising, from top to bottom:

a bottomless multi-well plate comprising a plurality of bottomless wells;

a first micropatterned polymer layer attached to a bottom surface of the bottomless multi-well plate to form a plurality of adjacent wells, one or more of each pair of adjacent wells comprising a transparent polymer membrane placed within the one ore mover of each pair of adjacent wells;

a second micropatterned polymer layer comprising two or more holes that correspond to two or more adjacent wells, the second micropatterned polymer layer being attached to a bottom surface of the first micropatterned polymer layer, such that each hole of the second micropatterned polymer layer is aligned with the two or more adjacent wells in the first micropatterned polymer layer, one or more of each pair of adjacent wells comprising the transparent polymer membrane;

a microfluidic channel formed between the two adjacent wells that allows internal fluidic communication between the two adjacent wells;

one or more removable polymer plugs, each located at a top surface of each of the plurality of wells, and one or more tubes, each connected to the one or more polymer plugs;

a pump connected to a reservoir that removably connects to the tubes;

a transparent, optical grade glass layer attached to the bottom surface of the second micropatterned polymer layer that forms a bottom surface for the plurality of wells and that seals the multi-well plate perfusion culture device;

wherein one or more of the two adjacent wells is a culture chamber for culturing a population of cells; and

(b) A liquid culture medium that is flowable between the first adjacent well and the second adjacent well;

The model being characterized by circulation of the liquid medium from the first well into the second well and back to the first well through the microfluidic channel;

With respect to the multiwell plate-based perfusion culture device, according to one embodiment of the described invention, the device comprises a plurality of layers. According to some such embodiments, the multiwall plate-based perfusion culture device comprises a bottomless multi-well plate including a plurality of bottomless wells; a first micropatterned polymer layer comprising a plurality of transparent polymer membranes therein, a second micropatterned polymer layer comprising a plurality of holes therethrough, a third micropatterned polymer layer comprising a plurality of holes therethrough, one blank glass layer for use with plate readers; and a plurality of fluidic passages formed between the polymer membrane and the blank glass layer. The term “bottomless multi-well plate” as used herein refers to a multi-well plate without a bottom surface; and the term “bottomless wells” as used herein refers to wells of the multi-well plate without a bottom surface.

According to some embodiments, the device can comprise more than three micropatterned polymer layers.

According to some embodiments, the first micropatterned polymer layer is attached to a bottom surface of the bottomless multi-well plate such that each of the plurality of transparent polymer membranes corresponds to each of the plurality of wells when the number of the polymer membranes is equal to the number of the wells, wherein the second micropatterned polymer layer is attached to a bottom surface of the first micropatterned polymer layer such that each of the plurality of holes corresponds to each of the plurality of wells.

According to some embodiments, a polymer membrane is placed in every other well in the multiwell plate so that the number of the polymer membranes in the multiwall plate equals one-half of the number of the wells.

According to some embodiments, the third micropatterned polymer layer is attached to a bottom surface of the second micropatterned polymer layer such that each of the plurality of holes in the third micropatterned polymer layer corresponds to two adjacent wells, thereby creating a microfluidic channel between the two adjacent wells to allow internal fluidic communication between the two adjacent wells. According to some embodiments, the third micropatterned polymer layer is attached to a bottom surface of the second micropatterned polymer layer such that each of the plurality of holes in the third micropatterned polymer layer corresponds to more than two adjacent holes in the second micropatterned polymer layer.

According to some embodiments, the second micropatterned polymer layer is omitted, and the third micropatterned polymer layer is attached to the bottom surface of the first micropatterned polymer layer, such that each hole of the third micropatterned polymer layer corresponds to one or more adjacent polymer membranes in the first micropatterned polymer layer.

According to some embodiments, the microfluidic channel is 200 μm thick and 5 μm high.

According to some embodiments, one of the two adjacent wells is a culture chamber, which is used to culture cells or tissues; and the second adjacent well is an outlet chamber, which is used to direct the effluent streams to exit through the top of the device, wherein a first tubing attached to the culture chamber is an inlet and a second tubing attached to the outlet chamber is merely an outlet, thus providing re-circulation of liquid medium together with non-adherent cells between two chambers. According to some such embodiments, the outlet chamber may or may not contain a polymer membrane.

According to some embodiments, both of the two adjacent wells are culture chambers, which are used to culture different cells or tissues. For example, according to an embodiment wherein both of the two adjacent wells are culture chambers, and these culture chambers are used to screen samples for determining a patient's risk of developing GVHD, the first chamber is used to culture epithelial cells, and the second is used to culture dendritic cells. According to some embodiments, the tubing connected to the first culture chamber is an inlet and another tubing connected to the second culture chamber is an outlet; thus providing re-circulation of liquid medium together with non-adherent cells between the two chambers.

According to some embodiments, the blank glass layer provides optical access through the bottom of the chambers for cell characterization with plate readers. According to some embodiments, the blank glass layer is attached to a bottom of the third micropatterned polymer layer to seal the multi-well plate culture device thereby forming a bottom surface thereof for the plurality of wells. According to some embodiments, the blank glass layer is about 1.2 mm-thick.

According to some embodiments, instead of comprising a plurality of layers, the well plate-based perfusion culture device comprises one polymer substrate which has multiple layers of holes therein, a first layer of holes comprises a plurality of holes, each corresponding to a shape and size and location of each of the plurality of wells, and a second layer of holes comprises a plurality of holes, each corresponding to a size and location of every two adjacent wells, thereby allowing internally fluidly connection between every two adjacent wells. According to some embodiments, each of the plurality of holes in the first layer of polymer substrate further has a transparent polymer membrane attached thereto.

According to some embodiments, the polymer substrate is made from polymer extrusion molding.

According to some embodiments, the micropatterned polymer layers are made of a polymer, e.g., polydimethyl siloxane (PMDS), polystyrene or the like.

According to some embodiments, the multi-well plate, the micropatterned polymer layers, and the glass layer are bonded (meaning joined securely to each other, for example, by an adhesive, a heat process, or pressure) using oxygen plasma treatments.

According to some embodiments, the multi-well plate-based perfusion culture device further comprises a plurality of removable polymer plugs (meaning a piece of material used to stopper an aperture), each located at a top surface of each of the plurality of wells; and a plurality of tubes (meaning a hollow, elongated body), each connected to each of the plurality of polymer plugs. According to some embodiments, the removable polymer plugs are made of a polymer, e.g., PDMS, polystyrene, or the like. According to some such embodiments, the removable polymer plugs made of PDMS are made by soft lithography. According to some such embodiments, the removable polymer plugs made of polystyrene (PS) are made by PS extrusion and bonding.

According to some embodiments, the device further comprises at least one pump connected to at least one reservoir, which removably connects to the tubes, e.g., the first tube and the second tube. According to some such embodiments; the pump controls flow rate of recirculation of the liquid medium, for example, via one or more valves, into and out of the wells.

According to some embodiments, the tube that connects the two adjacent chambers at the top of the device is a U-shaped tubing, and flow of a liquid medium is driven by the difference between an amount of liquid medium inside chamber 1 and chamber 2 until equilibrium is established.

According to some embodiments, a method for culturing cells in the multiwall plate device comprises (a) providing a liquid medium into a first well that is fluidly connected to a second well, such that the liquid medium flows from the first well into the second well, which is the well adjacent to the first well through the microfluidic channel, and (2) recycling the liquid medium back to the first well through a reservoir and pump or a U-tube externally connecting the two wells at the top of the device. According to some embodiments, the liquid medium flows at a rate of about 10-50 μL/min. According to some embodiments, the multi-well plate comprises at least 6, at least 12, at least 24, at least 48, at least 96, at least 384 or at least 1536 wells. The wells may have dimensions substantially same as the dimensions of the wells in plate currently commercially available for commercially available readers and dispensers. According to some embodiments, the multi-well plate has a substantially rectangular shape appropriate for commercially available readers and dispensers. According to some embodiments, the multi-well plate can have a shape different from rectangular.

According to some embodiments, the multi-well plate may be constructed of polymeric materials. Exemplary polymers include, without limitation, hydrophilic polyethylenes, polystyrenes, polypropylenes, acrylates, methacrylates, polycarbonates, polysulfones, polyesterketones, poly- or cyclic olefins, polychlorotrifluoroethylene, and polyethylene therephthalate. According to some embodiments, the multi-well plate can be constructed of polystyrene. According to some embodiments, the multi-well plate may be constructed of inorganic polymer materials.

According to some embodiments, the transparent polymer membrane provides optical access through the bottom surface of the culture chambers for cell characterization with plate readers. According to some embodiments, the transparent polymer membrane anchors tissue cells and biomaterials. According to some embodiments, the transparent polymer membrane is a transparent polycarbonate (PC) membrane. According to some embodiments, the transparent polymer membrane is a polyethylene terephthalate (PET) membrane. According to some such embodiments, the PET membrane has an average pore size of 8 μm.

According to some embodiments, the micropatterned polymer layers are used to anchor placement of the polymer membranes within the wells of the device that comprise one or more culture chambers.

According to some embodiments, the micropatterned polymer layers are constructed of a polymer. According to some such embodiments, the micropatterned polymer layers are made of polydimethyl siloxane (PMDS) or polystyrene. According to some such embodiments, the micropatterned polymer layers made of PMDS are made by soft lithography. According to some such embodiments, the micropatterned polymer layers made of polystyrene (PS) are made by PS extrusion and bonding.

According to some embodiments, the device further comprises biocompatible non-living material formed into a three-dimensional structure comprising interstitial spaces, for example, nanofibers or microbeads that are placed on a top surface of the polymer membrane. According to some embodiments, the microbeads comprise a polymer. According to some such embodiments, the microbeads comprise polystyrene. According to some such embodiments, the microbeads comprise biphasic calcium phosphate (BCP).

According to some embodiments, the polymer membrane is coated with a nanofiber mesh. According to some such embodiments, the nanofiber mesh comprises an electrospun PCL/collagen mesh. According to some embodiments, the PCL/collagen mesh comprises a nanofiber matrix comprising a plurality of pores through which the population of T lymphocytes derived from the potential donor allogeneic to the recipient subject can pass. According to some embodiments, the nanofiber matrix comprising the plurality of pores mimics the basement membrane of epithelial tissue and supports viability of the intestinal epithelial cells derived from the recipient subject.

According to some embodiments, the multiwell plate-based microfluidic perfusion culture device is effective to model multi-cellular microenvironments. According to some embodiments, the multiwell plate-based microfluidic perfusion culture device is effective to model perfusion effects on cell interactions. According to some embodiments, the multiwell plate-based microfluidic perfusion culture device is effective to model perfusion-induced shear stress on cell responses.

This ex vivo tumor approach also provides a new avenue (1) for testing of personalized therapeutics for MM patients; (2) for evaluating new drugs without the need for costly animal models; (3) for eliminating ineffective or unnecessary MM therapies; (4) for minimizing toxicity in MM patients; (5) for minimizing costs associated with MM therapy; (6) for minimizing the development of MM drug resistance; and (7) for studying the biology of MM, including mechanism(s) responsible for drug resistance and relapse.

According to some embodiments, the described invention provides a method for selecting a patient-specific treatment for MM based on an ex vivo response of a patient's MM cells (PMMCs) to a therapeutic agent. According to some embodiments, the described invention provides a method for assessing resistance to a therapeutic agent based on an ex vivo response of a patient's MM cells (PMMCs) to the therapeutic agent.

Non-limiting examples of therapeutic agents include chemotherapeutic agents, corticosteroids, immunomodulating agents, proteasome inhibitors, histone deacetylase (HDAC) inhibitors, monoclonal antibodies and interferon. Examples of chemotherapeutic agents include, but are not limited to, melphalan, vincristine, cyclophosphamide, etoposide, doxorubicin, liposomal doxorubicin and bendamustine. Non-limiting examples of corticosteroids include dexamethasone and prednisone. Examples of immunomodulating agents include, but are not limited to, thalidomide, lenalidomide and pomalidomide. Non-limiting examples of proteasome inhibitors include bortezomib, carfilzomib and ixazomib. Examples of histone deacetylase (HDAC) inhibitors include, but are not limited to, panobinostat. Non-limiting examples of monoclonal antibodies include daratumumab and elotuzumab. Interferons can be naturally-occurring or synthetic. Examples of interferons include, but are not limited to, interferon-α, interferon-β, interferon-γ and interferon-λ.

According to some embodiments, the described invention provides a method for assessing therapeutic efficacy of a test therapeutic agent based on an ex vivo response of a patient's MM cells (PMMCs) to the therapeutic agent.

According to some embodiments, the ex vivo response of the patient's MM cells (PMMCs) to the therapeutic agent is cell viability. Cell viability can be measured, for example, by enzyme activity, cell membrane permeability, cell adherence, ATP production, co-enzyme production, nucleotide uptake activity. colony formation, crystal violet, tritium-labeled thymidine, 3-(4,5-dimethylthiazol-2-yl)-2,5-diphenyltetrazolium (MTT), (4-[3-(4-iodophenyl)-2-[4-nitrophenyl]-2H-5-tetrazolio]-1,3-benzene disulfonate) (WST), apoptosis, flow cytometry and the like.

According to some embodiments, the device of the described invention is effective to conserve the endosteum niche. According to some embodiments, the device of the described invention is useful in studying solid tumors that metastasize to bone through the endosteum niche. Examples of solid tumors that metastasize to bone include, but are not limited to, breast cancer and prostate cancer.

III. A Method for Testing of Personalized Therapeutics for MM Patients; (2) and for Evaluating New Drugs without the Need for Costly Animal Models.

According to some embodiments, the described invention provides a method for selecting a patient-specific treatment for multiple myeloma (MM) comprises:

(a) preparing an in vitro multiwell plate-based perfusion culture device, comprising, from top to bottom:

1. a bottomless multi-well plate comprising a plurality of bottomless wells;

2. a first micropatterned polymer layer attached to a bottom surface of the bottomless multi-well plate to form a plurality of adjacent wells, one or more of each pair of adjacent wells comprising a transparent polymer membrane placed within the one or more of each pair of adjacent wells;

3. a second micropatterned polymer layer comprising two or more holes that correspond to two or more adjacent wells, the second micropatterned polymer layer being attached to a bottom surface of the first micropatterned polymer layer, such that each hole of the second micropatterned polymer layer is aligned with the two or more adjacent wells in the first micropatterned polymer layer, one or more of each pair of adjacent wells comprising the transparent polymer membrane;

4. a microfluidic channel formed between the two adjacent wells that allows internal fluidic communication between the two adjacent wells;

5. one or more removable polymer plugs, each located at a top surface of each of the plurality of wells, and one or more tubes, each connected to the one or more polymer plugs;

6. a pump connected to a reservoir that removably connects to the tubes;

7. a transparent, optical grade glass layer attached to the bottom surface of the second micropatterned polymer layer that forms a bottom surface for the plurality of wells and that seals the multi-well plate perfusion culture device;

wherein

(i) one or more of the two adjacent wells is a cell culture chamber comprising a first well region including a first well and a second well region including a second well;

(ii) the microfluidic channel connects the first well region and the second well region with one another;

(iii) the first well is adapted to receive a therapeutic agent, the second well is adapted to receive a biological sample of cells; and

(iv) liquids, nutrients and dissolved gas molecules flow through the channel;

(b) constructing an ex vivo endosteal microenvironment perfused by nutrients and dissolved gas molecules by;

1. seeding a surface of the culture chamber of the device of (a) with
(i) microbeads;
(ii) osteocyte cells (OSTs); and
(iii) osteoblast cells (OSBs),

2. culturing the cells with a culture medium through the microfluidic channel for a time effective for the cells to form three-dimensional (3D) nodular structures that comprise a 3D-endosteal-like tissue;

(c) acquiring bone marrow mononuclear cells (BMMCs) comprising viable multiple myeloma cells (MMCs) from a subject;

(d) bringing the BMMCs comprising viable MMCs in contact with the endosteal microenvironment perfused by nutrients and gas molecules to seed the ex vivo endosteal microenvironment with the viable MMCs, the ex vivo endosteal microenvironment perfused by nutrients and gas molecules seeded with viable MMCs forming an ex vivo microenvironment effective to recapitulate spatial and temporal characteristics of a multiple myeloma cancer niche and to maintain viability of the MMCs from the subject; and (e) testing therapeutic efficacy of a therapeutic agent on the viable MMCs maintained by the endosteal microenvironment of (d) in the first well adapted to receive a therapeutic agent of (a) by

1. contacting the MMCs maintained by the endosteal microenvironment of (d) with a test therapeutic agent; and

2. comparing at least one of viability and level of apoptosis of the MMCs contacted with the test therapeutic agent to an untreated MMC control, and

(f) initiating therapy to treat the subject with the test therapeutic agent if the test therapeutic agent is effective to significantly reduce viability of the MMCs contacted with the test therapeutic agent or to increase apoptosis of the MMCs contacted with the test therapeutic agent compared to the untreated MMC control.

According to some embodiments, the microbeads are biphasic calcium phosphate (BCP) microbeads, polystyrene (PS) microbeads or a combination thereof. According to some embodiments, the microbeads range in diameter from about 20 μm to about 25 μm. According to some embodiments, the microbeads are about 20 μm in diameter. According to some embodiments, the microbeads are about 21 μm in diameter. According to some embodiments, the microbeads are about 22 μm in diameter. According to some embodiments, the microbeads are about 23 μm in diameter. According to some embodiments, the microbeads are about 24 μm in diameter. According to some embodiments, the microbeads are about 25 μm in diameter.

### III. A Method for Optimizing Donor Selection for Allogeneic Transplantation and for Predicting Risk of GVHD

According to some embodiments, the described invention provides a method for optimizing donor selection for allogeneic blood and marrow transplantation (BMT) therapy comprises, in order:

(a) acquiring a tissue sample from a recipient subject allogeneic to a potential donor of a BMT graft, the tissue sample comprising a population of primary intestinal epithelial cells comprising an intestinal epithelial cell-specific antigen;

(b) seeding the population of primary intestinal epithelial cells (IECs) of (a) in a first adjacent well of a multiwall plate-based perfusion culture device, the first adjacent well comprising a transparent polymer membrane, expanding the population in a first liquid medium containing ROCK inhibitor Y-27632 and an irradiated Swiss 3T3-J2 fibroblast feeder layer and generating a population of conditional reprogrammed intestinal epithelial cells (CRIECs) comprising the intestinal cell-specific antigen derived from the recipient subject;

(c) acquiring a population of T lymphocytes from the potential donor allogeneic to the recipient;

(d) seeding and expanding in a second adjacent well of the multiwall plate-based perfusion culture device the population of T lymphocytes derived from the potential donor of (c),

(e) co-culturing in a second liquid medium the CRIECs derived from the recipient subject in the first adjacent well and the T lymphocytes derived from the potential donor allogeneic to the recipient subject in the second adjacent well, the co-culturing being characterized by:

(i) the first adjacent well being fluidly connected to the second adjacent well so that the second liquid medium is flowable between the first adjacent well and the second adjacent well; and

(ii) an interaction between the population of CRIECs derived from the recipient subject and the population of T lymphocytes that is effective to generate alloreactive effector T lymphocytes derived from the potential allogeneic donor;

(f) measuring damage to the population of CRIECs derived from the recipient subject induced by the alloreactive effector T lymphocytes derived from the potential donor allogeneic to the recipient subject, wherein the damage is a measure of a risk of intestinal graft versus host disease in the recipient subject;

(g) ranking a plurality of potential donors by the measure of the risk of intestinal graft versus host disease; and

(h) treating the recipient subject with a BMT graft derived from a selected donor allogeneic to the recipient subject whose T lymphocytes are characterized by a reduced risk of intestinal graft-versus-host disease.

According to some embodiments, the sample is a biopsy sample. According to some embodiments, the biopsy sample is a small biopsy sample of the order of 3 mm3. According to some embodiments, the biopsy sample is collected from intestinal tissue. According to some embodiments, the biopsy sample is collected from intestinal tissue by colonoscopy, endoscopy, or a combination thereof.

According to some embodiments the potential donor is a haploidentical donor (i.e., parent, child and other close relative),

According to some embodiments, the patient sample is acquired soon after diagnosis of a hematological malignancy for which allogeneic BMT is a potential therapeutic approach and stored for later use in the method. According to some embodiments, the patient sample is acquired in the relapse setting after chemotherapeutic interventions have been exhausted.

According to some embodiments, any cells of interest may be cultured. According to some embodiments, the cells to be cultured can include normal, diseased, stem, cancerous, and/or mutated cells.

According to some embodiments, the primary IECs are prepared from the small intestine, large intestine or colon of the recipient subject, and expanded using conditional reprogrammed cell technology, which comprises cultivating the primary IECs in a medium containing ROCK inhibitor Y-27632 and an irradiated Swiss 3T3-J2 fibroblast feeder layer. According to some embodiments the medium for cultivating the primary human IECs containing ROCK inhibitor Y-27632 and an irradiated Swiss 3T3-J2 fibroblast feeder layer is RPMI.

While expansion of conditionally reprogrammed cells is useful in expanding the IECs from biopsy samples, CRCs cannot be used for co-culture of CRIECs and T cells for 2-3 weeks due to adverse effects of CRC media additives (e.g., ROCK kinase inhibitor) on T cell motility and functionality. (Riento, et al., Molecular cell biology (2003) 4, 446-456; Iyengar, et al., Journal of the American Society for Blood and Marrow Transplantation, doi:10.1016/j.bbmt.2014.04.029 (2014)). According to some embodiments, the CRC medium is replaced with a complete RPI-1640 medium (defined as RPMI-1640 supplemented with 10% fetal bovine serum and 5% L-glutamine) to culture the T cells.

In native tissues, IECs reside on the thin fibrous basement membrane (BMa) consisting of intermingled networks of laminins and type IV collagen and provide cell anchoring and barrier functions. The membrane networks interact with cells through membranous integrin receptors and other plasma membrane molecules, influencing cell differentiation, migration, adhesion, phenotype and survival.

According to some embodiments, the first well comprises a nanofibrous coated transparent polymer membrane. According to some embodiments, the nanofibrous coating is prepared by electrospinning. According to some embodiments, the nanofibrous coating comprises a fiber matrix of polycaprolactone in which extracellular matrix (ECM)-like molecules (e.g., collagen) is dispersed. According to some embodiments, the nanofibrous coated transparent polymer membrane is effective to maintain the long term functionality of CRIECs and T cells using RPMI as a common culture medium. Wang's prior research (Fu, et al. Biomaterials (2014) 35, 1496-1506) has shown that, as a result of mimicking the morphological and dimensional characteristics of base membrane extracellular matrix (ECM) fibrils, nanofibrous meshes can support keratinocytes to form skin-like structures and maintain cobble stone-like morphology for 2 weeks (Huang, et al., Biomaterials (2012) 33, 1791-1800).

According to some embodiments, the method comprises providing polymer microbeads preconditioned with one or more adhesion-promoting agents to promote adhesion of cells to at least one surface of the microbeads. According to some such embodiments, the cells are dendritic cells (DCs). According to some embodiments, the adhesion promoting agent comprises an effective amount of lipopolysaccharides (LPS), wherein the LPS are effective to promote adhesion of the DCs to the microbeads surface.

According to some embodiments, the first well of the multiwall plate device contains a population of conditionally reprogrammed IECs prepared from a mammalian subject, and the second well fluidly connected to the first well contains T cells comprising dendritic cells from a mammal allogeneic to the mammalian subject. According to some such embodiments, the mammal is a mouse. According to some such embodiments, the mammal is a human.

According to some embodiments, a CRIEC culture chamber can be established by placing CRIECs into the first well on top of a polymer membrane coated with an electrospun PCL/collagen nanofibrous mesh.

According to some embodiment, an average open space (or pore size) in the nanofibrous mesh is within a range of about 1-10 μm. According to some such embodiments, the average open space (or pore size) in the nanofiber mesh is about 1, 2, 3, 4, 5, 6, 7, 8, 9, or 10 μm.

According to some embodiments, an antigen presenting cell (APC) culture chamber can be established by placing T cells comprising DCs in the liquid medium onto the pre-treated polymer microbeads in the second well. According to some embodiments, one or more cytokines can be added to the culture chamber to prolong T cell maintenance. According to one embodiment, the population of T-cells suspended in the liquid medium comprises about 105 to 106 cells.

According to some embodiments, the method further comprises replenishing DCs with new DCs by opening a polymer plug on the top of the APC chamber and placing new DCs onto the top of the microbead assembly. According to some embodiments, the dendritic cell assembly can be replaced by a new microbead/dendritic cell assembly.

According to some embodiments, an average size of a polymer microbead is in a range of about 45-90 μm. According to some such embodiments, the average size of a polymer microbead is about 45, 50, 55, 60, 65, 70, 75, 80, 85, or 90 μm.

According to some embodiments, the T cells comprising dendritic cells are derived from peripheral blood lymphocytes. According to some embodiments, mouse dendritic cells are enriched by injecting host mice with a B16-FLt3L tumor.

According to some embodiments, the polymer membrane has an average pore size that provides a sufficient opening for T cells to go through. According to some such embodiments, the average pore size of the polymer membrane is about 7-13 μm. According to some such embodiments, the average pore size of the polymer membrane is about 7, 8, 9, 10, 11, 12 or 13 μm. According to some such embodiments, an average diameter of a T cell is about 5 μm.

According to some embodiments, the nanofibrous coated transparent polymer membrane is effective to anchor a population of cells. According to some embodiments, the polymer membrane comprises the population of human intestinal epithelial cells, the population of CRIECs, or a combination thereof.

According to some embodiments the cells to be cultured can be cultured in free suspensions, encapsulated in suitable hydrogels, encapsulated in matrices, and/or encapsulated in scaffolds. For example, according to some embodiments, the T cells comprising a suspension of about 106 T cells (e.g., eGFP m-T cells (harvested from an eGFP transgenic B6 mouse) or h-T cells) in a culture medium are flowable, i.e., they circulate with the liquid medium of the microfluidic well plate-based perfusion culture device. According to one embodiment, the culture medium contains retinoic acid, which facilitates the generation of T cells with superior IEC-killing avidity.

According to some embodiments, a ratio of CRIECs: T cells is in a range of from 2:1 to 20:1. According to some such embodiments, the ratio of CRIECS: T cells is 2:1, 34:1, 4:1, 5:1, 6:1, 7:1, 8:1, 9:1, 10:1, 11:1, 12:1, 13:1, 14:1, 15:1, 16:1, 17:1, 18:1, 19:1, or 20:1.

According to some embodiments, a ratio of T cells: DCs is in a range of from 1:1 to 20:1. According to some such embodiments, the ratio of T cells: DCs is 1:2, 2:1, 3:1, 4:1, 5:1, 6:1, 7:1, 8:1, 9:1, 10:1, 11:1, 12: 1, 13:1, 14:1, 15:1, 16:1, 17:1, 18: 1, 19:1, or 20:1. According to some such embodiments, the ratio of T cells: DCs is 5:1.

According to some embodiments, the co-culturing of the population of human IECs from the proposed recipient subject and the population of T lymphocytes from the donor allogeneic to the recipient subject is effective to generate alloreactive activated T-lymphocytes. According to some embodiments, the allogeneic activated/effector T cells recirculate through interconnected h-CRIEC and antigen presenting cell (APC) culture chambers. According to some embodiments, the alloreactive activated/effector T-lymphocytes comprise a population of antigen presenting cells. According to some such embodiments, the population of antigen presenting cells comprises a population of dendritic cells. According to some embodiments, the alloreactive T cells become activated by cognitive alloantigens on h-CRIECs. According to some embodiments, the alloreactive activated/effectorT cells comprise activated antigen presenting cells (APCs). According to some embodiments, the APCs comprise activated/effector dendritic cells. According to some embodiments, the alloreactive activated/effector T cells are effective to induce quantifiable damage to the population of h-CRIECs. According to some such embodiments, the device is clinically viable, i.e., it is effective to increase the critical number of functional T cells required to induce quantifiable alloreactivity in the CRIEC culture chamber within a diagnostic screening time frame of 2-3 weeks.

According to some embodiments, quantifiable damage to the population of CRIECs comprises measurable killing of the population of CRIECS. According to some embodiments, a pathological index (PIdx) is used to quantify T cell induced CRIEC damage. For example, the predictive capability of co-culture killing assays can be compared to known in vivo outcomes from well-established murine models of BMT.

According to some such embodiments, a panel of cell death analysis methods is used to quantify cell death. For example, annexin V/PI staining using flow cytometry and in situ detection of cleaved caspase-3 using immunofluorescence can be used to determine cell death. According to some embodiments, the percentage of dead cells is calculated as [% of Annexin V+/PI+ cells in co-cultures−% of Annexin V+/PI+ cells in IEC alone cultures] (flow cytometric measurement). According to some embodiments, CD3 staining is performed to identify adherent T cells contributing to the response. According to some embodiments, the caspases-3 staining is conducted in the well plate to determine cell death as [fluorescence intensity in co-culture−fluorescence intensity in IEC alone cultures]/[fluorescence intensity of DAPI staining, as an indicator of the number of nucleated cells in the cultures].

According to some embodiments, cell death is evaluated at three or more T cell-IEC ratios (i.e., the effector: target, or E:T ratio). According to some embodiments, the E:T ratio is 30, 10, or 3. See Choksi, S. et al, “A cD8 DE loop peptide analog prevents graft versus host disease in a multiple minor histocompatibility antigen-mismatched bone marrow transplantation model,” Biology of Blood and Marrow Transplantation: 10: 669-680, doi: 10.1016/j.bbmt.2004.06.005 (2004)).

According to some embodiments, the PIdx can be determined as the slope of the curve of percentage of dead cells vs. E:T ratios, where a steeper curve indicates a higher risk for developing GVHD. According to some embodiments, the PIdx can be determined at multiple time points post-co-culture.

According to some embodiments, the cells are cultured only in the first well, and the connected adjacent second well is an outlet well providing exit of the liquid medium from the top of the device.

According to some embodiments, cells of different types may be cultured at the same time in different fluidly connected wells of the plate-based perfusion device. For example, a first cell type can be seeded in and cultured in the first well while a second cell type can be seeded in and cultured in the second well at the same time.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the described invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Isolation, Characterization and Preservation of Primary Human Osteocytes (ph-OSTs)

Thirty (30) bone cores, bone marrow (BM) aspirates, and peripheral blood will be obtained with informed consent. Also, 30 existing patient samples, which were collected and stored through informed consent, will be utilized for the development of the tissue models.

Primary human osteocytes (ph-OSTs) will be isolated from discarded bone samples generated during hip joint replacement surgeries (20 samples in total). These samples will be used: (1) to avoid potential complications from subjecting already significantly damaged bone cells in multiple myeloma (MM) patient bone core samples to stringent bone digestion procedures, (2) because a higher number of osteocytes (OSTs) can be isolated from larger surgical samples, and (3) because of our ability to recapitulate the osteolytic effects of MM on ossified tissues grown with osteoblasts (OSBs). If necessary, ph-OSTs will be isolated from MM bone core biopsies.

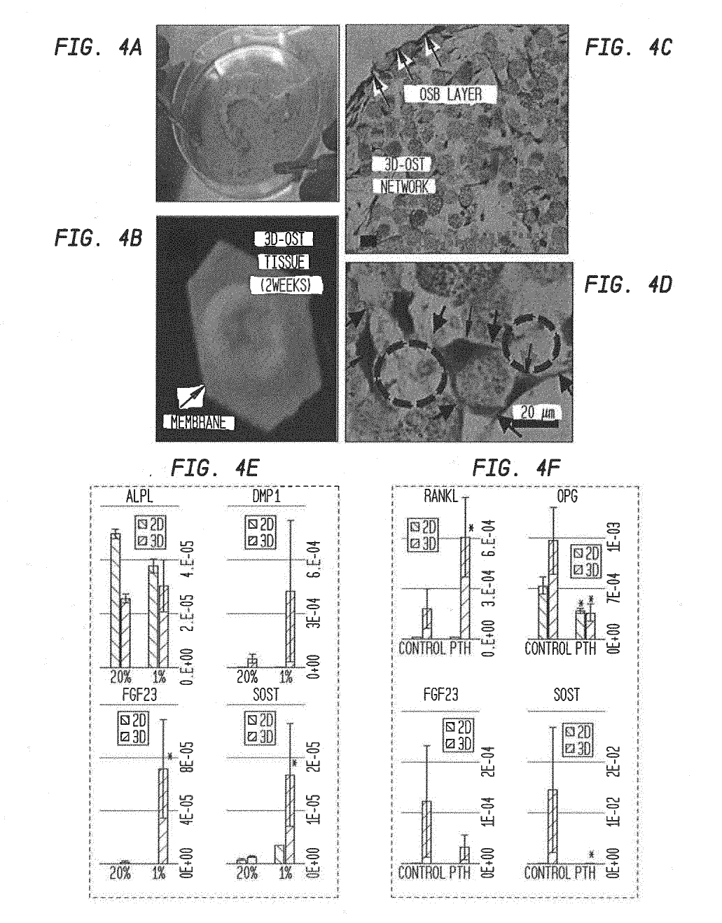

Briefly, the samples from discarded bone generated during hip joint replacement surgeries (FIG. 4A) will be cut into 3 mm chips and subjected to a series of collagenase and ethylenediaminetetraacetic acid (EDTA) digestion steps (Stern, A. R. et al. Isolation and culture of primary osteocytes from the long bones of skeletally mature and aged mice. BioTechniques 52, 361-373, doi:10.2144/0000113876 (2012); Stern, A. R. & Bonewald, L. F. Isolation of osteocytes from mature and aged murine bone. Methods in molecular biology 1226, 3-10, doi:10.1007/978-1-4939-1619-1_1 (2015).). After the seventh digestion, the bone chips will be washed, plated, and submerged in Eagle's minimum essential medium with alpha modifications (α-MEM) supplemented with 10% fetal bovine serum for bone cells to migrate out and grow for 8 days. The osteocytic nature of outgrown cells will be confirmed by immunostaining for sclerostin and alkaline phosphatase (ALP). In order to reduce the effects of sample-to-sample variability, isolated ph-OSTs will be cryopreserved. After acquiring sufficient samples, cells from at least 10 different subjects will be thawed, pooled and cryopreserved until needed for tissue reconstruction and culture experiments. Cells will be freeze-thawed no more than two times before use, to minimize any potential cell damage due to the preservation procedures. For experiment-to-experiment consistency, the osteocytic nature of ph-OSTs will be evaluated and confirmed before and after each experimental campaign using: (1) immunostaining of ALP, Ell, and sclerostin (SOST) and (2) gene expressions of alkaline phosphatase, liver/bone/kidney lysozyme (ALPL), podoplanin (PDPN) (Ell), dentin matrix acidic phosphoprotein 1 (DMP1), SOST, and fibroblast growth factor 23 (FGF23).

### Example 2. 96-Well Plate-Based Culture Platform for 3D Tissue Reconstruction

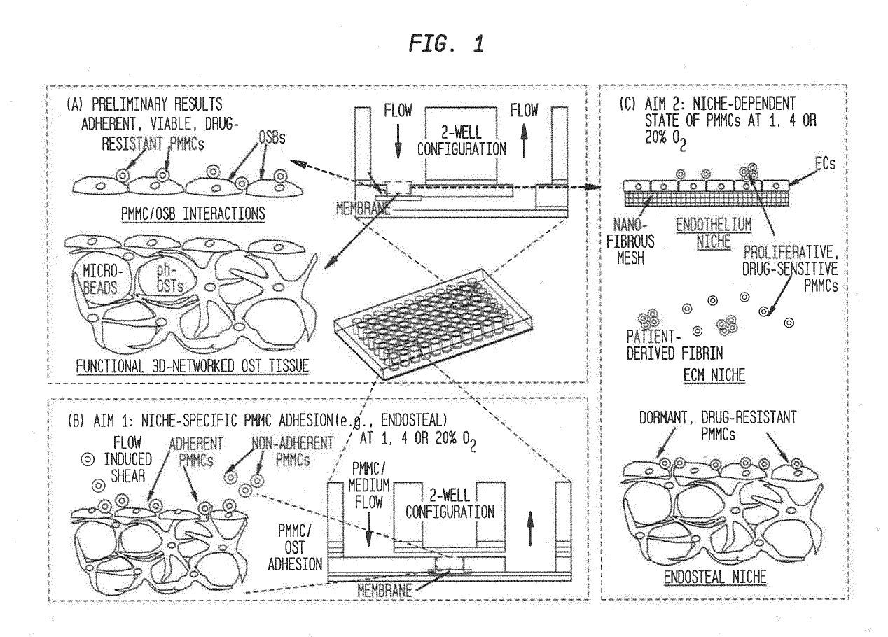

As depicted in FIG. 1, a prototype device was fabricated using a commercially available polystyrene 96-well plate with a polydimethylsiloxane (PDMS) bottom. Two wells were used to produce one (1) culture chamber and one (1) outlet chamber so that 48 wells were available for 3D culture. The outlet chamber was used to direct the effluent streams to exit through the top of the device so that the device bottom could be preserved for optical reading. FIG. 1 shows the micropatterned polymer layers that were used to: (1) provide a microfluidic channel of 200 μm thick and 5 mm wide between the culture and outlet chambers; and (2) anchor the placement of a polymer membrane in the culture chamber during assembly of the device. 3D tissue and tumor structures can be reconstructed on the membrane surface with or without using biomaterials.

Unlike conventional 96-well tissue culture plates, the transparent polymer membranes enable perfusion culture while being able to hold tissue cells and biomaterials and providing optical access through the bottom of the culture chambers. Moreover, the use of removable polymer plugs at the top of the culture chambers enables (1) convenient placement of cells and biomaterials into the culture chambers at various time points during culture and (2) a fluid connective link between a multiple number of culture chambers established by external tubing and a pump so that non-adherent cells such as immune cells and circulating tumor cells can be circulated through the chambers.

### Example 3. Patient Multiple Myeloma Cell (PMMC)-Osteoblast (OSB) Interactions

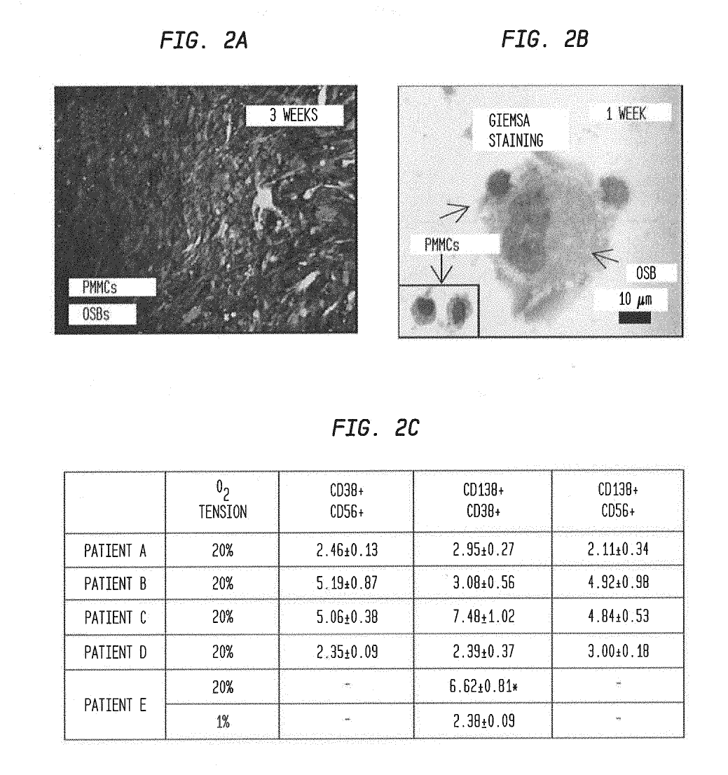

Using the 96-well plate-based culture platform described in Example 2, osteoblasts (OSBs) (hFOB 1.19 cell line) were cultured for 4 days at 20% O₂ tension to form an ossified tissue structure. Patient bone marrow mononuclear cells (BMMCs) containing PMMCs, were seeded on the ossified tissue structure and cultured for up to 5 weeks. Results indicated that: (1) various PMMC populations attached to OSBs, remained viable, and proliferated significantly during a 3-week culture period (FIG. 2A), whereas patient multiple myeloma cells (PMMCs) underwent apoptosis within a few days of 2D culture (FIG. 2B); (2) proliferating PMMCs gradually compromised the viability of OSBs; (3) the viability of PMMCs slowly decreased over 3 weeks due to the loss of OSBs; and (4) long-term survival of both OSBs and PMMCs were enhanced by stimulating OSBs with optimized perfusion flow rate and replenishing OSBs during the culture. When O₂ tension was decreased to 1%, the proliferation rate of PMMCs decreased (FIG. 2C) while their viability was not compromised (data not shown). In addition, adhesion between PMMCs and OSBs: (1) was mediated in part by osteoblastic N-cadherin using genetically modified human hFOB 1.19 cells with downregulated N-cadherin expression; and (2) was required to maintain ex vivo viability and proliferative capacity of PMMCs, whereas conditioned medium from OSB culture was not sufficient. PMMCs also acquired cell adhesion mediated drug resistance (CAM-DR) due to the adhesive interactions with OSBs.

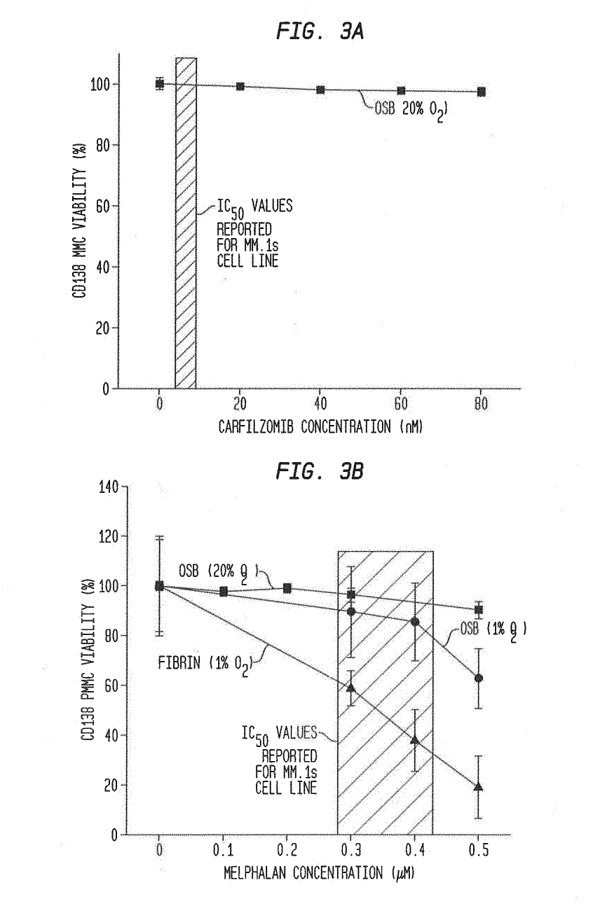

The response of PMMCs to two commonly used drugs to treat MM patients: (1) carfilzomib, a proteasome inhibitor; and (2) melphalan, which is often used to treat newly diagnosed MM patients along with stem-cell transplantation, was evaluated. OSB tissue was cultured for 4 days either at 1 or 20% O2 prior to seeding of PMMCs. After seeding, PMMC-OSB cultures were dosed with either carfilizomib or melphalan. After 24 h, flow cytometry was used to analyze the viability of CD138+PMMCs. As shown in FIG. 3A, CD138+ PMMCs exhibited significant resistance to 100 nM carfilzomib at 20% O2, in comparison to IC50 values of 6 to 8.4 nM reported for an MM.1S cell line (Hurchla, M., Garcia-Gomez, A., Hornick, M., Ocio, E., Li, A., Blanco, J., Weilbaecher, K. The epoxyketone-based proteasome inhibitors carfilzomib and orally bioavailable oprozomib have anti-resorptive and bone-anabolic activity in addition to anti-myeloma effects. Leukemia 27, 430-440, doi:10.1038/leu.2012.183 (2013); Shannon M. Matulis, S. H., Cathy Sharp, Francis Burrows, Ajay K. Nooka, Jonathan L. Kaufman, Sagar Lonial, Lawrence H. Boise. Dual Inhibition Of Mcl-1 By The Combination Of Carfilzomib and TG02 In Multiple Myeloma, (2013)). When cultured with OSBs at both 1 and 20% O2, CD138+PMMCs were highly resistant to 100 μM melphalan in comparison to IC50 of 2 to 40 μM reported for the cell line (FIG. 3B) (Dharminder Chauhan, M. B., Ajita V Singh, Klaus Podar, Paul G. Richardson, Nikhil C. Munshi, Kristina Viktorsson, Rolf Lewensohn, Jack Spira and Kenneth C. Anderson. Anti-Myeloma Activity of Enzymatically Activated Melphalan Prodrug J1. 53rd ASH annual meeting and exposition (2011); Kuhn, D. J., Kornblau, S. M., Wang, M., Weber, D. M., Thomas, S. K., Shah, J. J., Voorhees, P. M., Xie, H., Cornfeld, M., Nemeth, J. A. and Orlowski, R. Z. Blockade of interleukin-6 signalling with siltuximab enhances melphalan cytotoxicity in preclinical models of multiple myeloma. British journal of haematology 152, 579-592, doi:10.1111/j.1365-2141.2010.08533.x. (2011); Xiang, Y. et al. Monitoring a Nuclear Factor-κB Signature of Drug Resistance in Multiple Myeloma. Molecular & Cellular Proteomics 10, doi:10.1074/mcp.M110.005520 (2011)). In contrast, CD138+PMMCs cultured in fibrin were significantly more sensitive to melphalan. Without being bound by theory, the data suggests that adhesion to OSB dominates the effect of O2 tension on the development of drug resistance.

These results demonstrate that: (1) the 96-well plate-based 3D culture platform recapitulates the in vivo survival and drug resistance of PMMCs supported by OSBs; (2) O2 tension can significantly control the proliferation of PMMCs; and (3) the adhesion of PMMCs to OSBs may dominate over the potential effect of O2 tension in the manifestation of drug resistance.

### Example 4

4.1 Reconstruction of a 3D Cellular Network Found in Native Bones Via Biomimetic Assembly of Osteocytes and Microbeads in the Microfluidic Perfusion Culture Device.

A functional 3D osteocyte network can be reconstructed by the biomimetic assembly of osteocyte cell line MLO-A5 (murine osteocyte-like cell line, Kerafast, Inc.) cells and microbeads within the physical confines of microfluidic culture chambers, as illustrated in FIG. 1.

FIG. 4 shows a three dimensional (3D)-networked primary human osteocyte (ph-OST) reconstruction at 1% O2. FIG. 4A shows a human bone sample. Cells and biphasic calcium phosphate (BCP) or polystyrene (PS) microbeads in the range of 20-25 μm (i.e, 20 μm, 21 μm, 22 μm, 23 μm, 24 μm and 25 μm) in diameter were assembled into the culture chamber, via forced liquid withdrawal through the PC membrane, to form a close-packed structure (FIG. 1). The microbead diameter in the range of 20-25 μm (i.e., 20 μm, 21 μm, 22 μm, 23 μm, 24 μm and 25 μm) was used: (1) to distribute and entrap cells within the interstitial spaces between the microbeads; and (2) to maintain average cell-to-cell distance in the range of 20 to 30 μm (FIG. 4D), similar to that observed in human bones (Hannah, K. M., Thomas, C. D., Clement, J. G., De Carlo, F. & Peele, A. G. Bimodal distribution of osteocyte lacunar size in the human femoral cortex as revealed by micro-CT. Bone 47, 866-871, doi:10.1016/j.bone.2010.07.025 (2010)). The cells entrapped within the interstitial spaces between the microbeads formed a 3D cellular network by extending and connecting their processes through openings between the microbeads over 2 weeks. These cells did not proliferate due to spatial confinement and produced a mineralized ECM to form a mechanically integrated tissue structure (FIG. 4B). Similar network formation, non-proliferative, and extracellular matrix (ECM) production behaviors were observed at 20% O2 (data not shown). The 3D-reconstructed cells exhibited increased gene expressions (DMP1, SOST, and FGF23) characteristic of mature osteocytes at 1% O2 compared to 3D-reconstructed cells at 20% O2 (FIG. 4E). In contrast, 2D-cultured primary OSTs proliferated significantly and underwent significant osteoblastic differentiation, as evidenced by increased ALPL gene expression and negligible detection of the osteocytic gene expressions (e.g., DMP1, SOST, and FGF23). The observed O2 tension effects are similar to those reported on the osteocytic differentiation of OSBs (Hirao, M. et al. Oxygen tension is an important mediator of the transformation of osteoblasts to osteocytes. Journal of bone and mineral metabolism 25, 266-276, doi:10.1007/s00774-007-0765-9 (2007); Chen, D. et al. HIF-1alpha inhibits Wnt signaling pathway by activating Sost expression in osteoblasts. PloS one 8, e65940, doi:10.1371/journal.pone.0065940 (2013); Clinkenbeard, E. L. et al. Neonatal iron deficiency causes abnormal phosphate metabolism by elevating FGF23 in normal and ADHR mice. Journal of bone and mineral research: the official journal of the American Society for Bone and Mineral Research 29, 361-369, doi:10.1002/jbmr.2049 (2014)). Notably, a layer of OSBs formed on the unconfined surface of the 3D-OST tissue, replicating an osteoblastic endosteal layer found in vivo (FIG. 4C).

In order to characterize the regulatory functions of 3D-osteocytes (OSTs) on osteoclastogenesis and osteoblastic development, the effects of continuous parathyroid hormone (PTH) treatment was evaluated. PTH is known to induce OSTs to increase gene expression of receptor activator of nuclear factor kappa-B ligand (RANKL), decrease gene expression of osteoprotegerin (OPG), and decrease gene expression of sclerostin (SOST) (Bonewald, L. F. The amazing osteocyte. Journal of bone and mineral research: the official journal of the American Society for Bone and Mineral Research 26, 229-238, doi:10.1002/jbmr.320 (2011); Bellido, T., Saini, V. & Pajevic, P. D. Effects of PTH on osteocyte function. Bone 54, 250-257, doi:10.1016/j.bone.2012.09.016 (2013). 3D-OSTs that were treated with 50 nM PTH for 2 days exhibited increased RANKL gene expression, decreased OPG gene expression, decreased SOST gene expression and decreased FGF23 gene expression compared to no treatment and 2D-OSTs controls (FIG. 4F).

These results demonstrate that the ex vivo 3D reconstruction approach is effective to preserve the in vivo phenotype and functions of terminally differentiated and 3D-networked ph-OSTs residing in bone tissue; and to replicate the formation of an endosteal primary human osteoblast (ph-OSB) layer on the tissue surface, which in turn, show the feasibility of reconstructing a 3D cellular network with primary osteocytes in the multiwell plate-based microfluidic perfusion culture device of the described invention.

4.2 Adhesive Interactions Between Patient Multiple Myeloma Cells (PMMCs) and Osteocytes (OSTs).

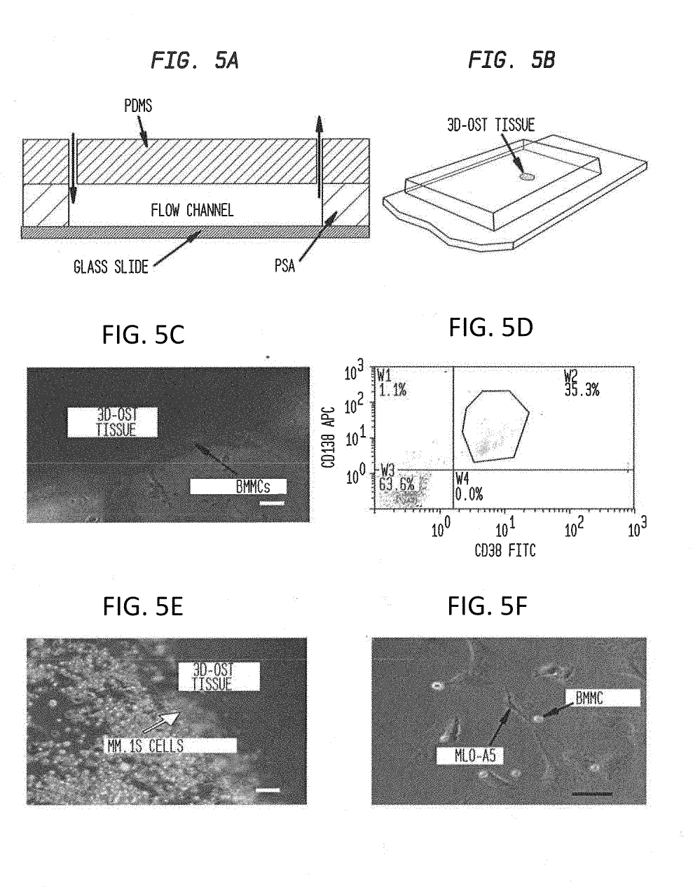

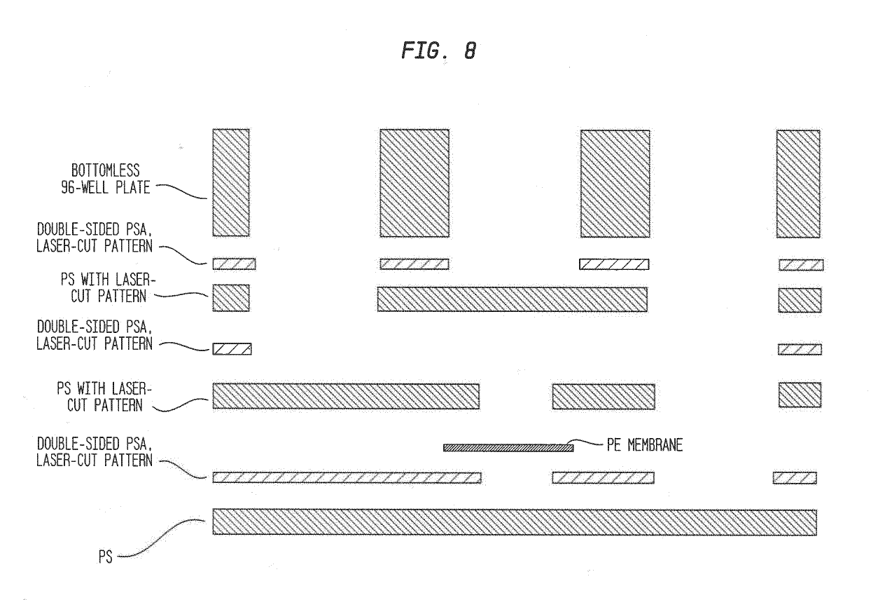

FIGS. 5A-5B and 8 show a version of the well plate-based culture platform used in this study. Three (3) double-sided, biocompatible, and pressure-sensitive adhesive (PSA) layers (ARcare 90106, Adhesive Research) and a phosphatidylethanolamine (PE) membrane layer with an average pore size of 1 μm (Sterlitech) were patterned and cut using CO2 laser by a vendor (Las X Industries) and assembled with a bottomless 96-well plate and a blank polystyrene (PS) layer to fabricate the culture platform. The PSA consisted of a 25 μm-thick PE layer sandwiched by 60 μm-thick layers of medical grade acrylic hybrid adhesive. Patient BMMCs pre-labelled with CellTrace violet (ThermoFisher) and MM.1S (B lymphoblast cell line; ATCC) cells were introduced to the culture device containing the 3D-OST tissue constructed with MLO-A5 cells and PS microbeads. After overnight culture, non-adherent cells were flushed using a high medium flow rate (100 μL/min). Cell adhesion was monitored by time-lapse microscopy for 10 min using the medium flow rate of 0.8 μL/min. Under these conditions, BMMCs and MM.1S cells adhered to the 3D tissue surface (FIGS. 5C and 5E). BMMCs also adhered to 2D-cultured MLO-A5 cells (FIG. 5F). After 5 days in culture, the presence of CD138+CD38+ PMMCs among the adhered BMMCs was identified by flow cytometry using CD138-PC5 and CD38-FITC antibodies (FIG. 5D).

These observations are consistent with recently reported in vivo and in vitro results showing that JJN3 cells (a human MM cell line) interacts adhesively with OSTs to adversely alter the gene expressions of OSTs associated with bone remodeling regulation (Delgado-Calle, J. et al. Bidirectional Notch Signaling and Osteocyte-Derived Factors in the Bone Marrow Microenvironment Promote Tumor Cell Proliferation and Bone Destruction in Multiple Myeloma. Cancer research 76, 1089-1100, doi:10.1158/0008-5472.CAN-15-1703 (2016)).

These results show that PMMCs directly adhere to the 3D OST tissue constructed with MLO-A5 cells and suggest that PMMCs are likely to adhere to the 3D tissue constructed with ph-OSTs.

### Example 5. Fibrin Extracellular Matrix (ECM)

In this study, fibrin was used to reconstruct extracellular matrix (ECM) niche tissue containing patient bone marrow mononuclear cells (BMMCs). The advantages of using fibrin as opposed to other BM-mimicking ECM materials (e.g., collagen gel, alginate gel, Matrigel®, polymer and hydroxyapatite) include long-term viability of patient-derived BMMCs and PMMCs; the ability to use patient plasma for reconstructing patient-based ECM; sufficient transparency for 3D imaging; and tailorable interconnected pore size distribution for cell migration (de la Puente, P. et al. 3D tissue-engineered bone marrow as a novel model to study pathophysiology and drug resistance in multiple myeloma. Biomaterials 73, 70-84, doi:10.1016/j.biomaterials.2015.09.017 (2015); Bara, J. J. et al. Three-dimensional culture and characterization of mononuclear cells from human bone marrow. Cytotherapy 17, 458-472, doi:10.1016/j.jcyt.2014.12.011 (2015); Chiu, C. L., Hecht, V., Duong, H., Wu, B. & Tawil, B. Permeability of three-dimensional fibrin constructs corresponds to fibrinogen and thrombin concentrations. BioResearch open access 1, 34-40, doi: 10.1089/biores.2012.0211 (2012)).

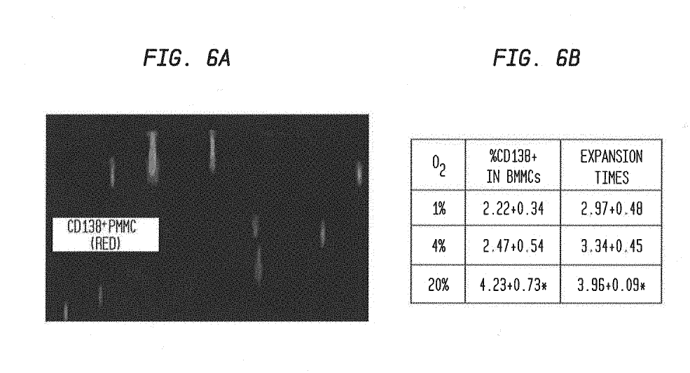

FIG. 6A shows a reconstructed 2D view of 3D cross-sectional confocal images from CD138⁺PMMCs, 1.2% among patient BMMCs, dispersed in fibrin matrix prior to culture. FIG. 6B shows that fibrin supports the viability and proliferation of CD138⁺PMMCs as characterized by flow cytometry and also shows that there was no significant difference in proliferation of PMMCs when cultured in the fibrin ECM at 1% O₂ or 4% O₂ tension.

### Example 6. Endothelium Construction

In this study, electrospun nanofibers were used to develop a functional endothelium with controlled permeability. Recently, electrospun nanofibers have been found to morphologically and dimensionally mimic the native basement membrane functions of endothelium basement membrane and to maintain the phenotype of endothelial cells (ECs) in vitro (Chen, X. et al. Shell-core bi-layered scaffolds for engineering of vascularized osteon-like structures. Biomaterials 34, 8203-8212, doi:10.1016/j.biomaterials.2013.07.035 (2013)).

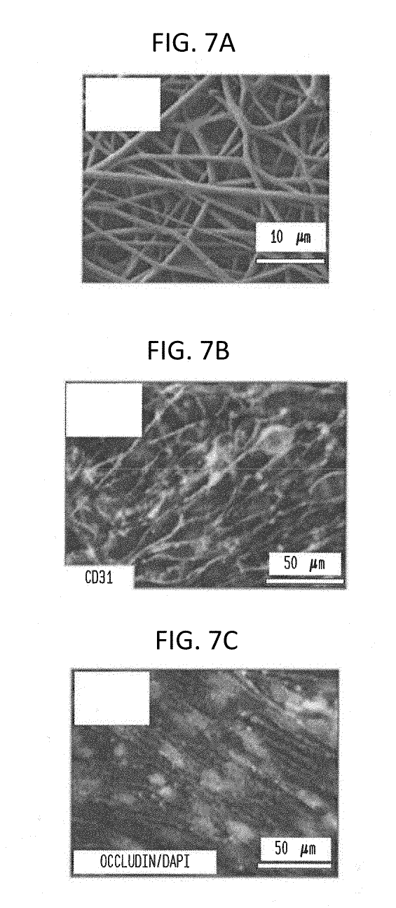

MS1 cells, a mouse endothelial cell line (ATCC), were cultured on a polycaprolactone (PCL)/collagen electrospun nanofiber mesh with a fiber diameter of 200-600 nm. FIG. 7A shows that the MS1 cells rapidly attached and spread on the mesh surface to form a confluent layer. CD31 expression, a marker of intercellular junctions, showed the formation of a confluent cell layer (FIG. 7B). Occludin expression further demonstrated the formation of tight junction between adjacent ECs (FIG. 7C). The formation of tight junctions has been shown to be critical in developing a functional endothelium with controlled permeability (Hirase, T. et al. Occludin as a possible determinant of tight junction permeability in endothelial cells. Journal of cell science 110 (Pt 14), 1603-1613 (1997)).

### Example 7. Adhesive Interactions of Patient Multiple Myeloma Cells (PMMCs) with Endosteal Tissue Surface and Extracellular Matrix (ECM)

7.1 Cell Culture Platform Design

A 3D-osteocyte (OST) tissue containing a 2D-osteobalst (OSB) surface layer (FIGS. 1 and 4C) will be reconstructed to determine specific PMMC populations that adhere to OSTs and/or OSBs. Three (3) wells of the well plate platform (FIG. 8) will be used to produce the endosteal tissue surface onto which adherent PMMCs can attach. Non-adherent PMMCs can be removed by flow-induced shear stress for downstream analysis. The PE membrane shown in FIG. 1 is required to closely pack PS microbeads and OSTs via forced withdrawal of the suspension medium prior to sealing the culture platform as illustrated in FIG. 8. The 3D-OST tissue will be cultured statically for 1 week prior to the seeding of BMMCs.

Similarly, fibrin-based and endothelium tissue surfaces will be constructed to mimic bone marrow (BM) ECM and BM vasculature, respectively (FIG. 1). Fibrin gel will be developed through cross-linking of fibrinogen (naturally found in patient plasma) with CaCl2 as previously described (de la Puente, P. et al. 3D tissue-engineered bone marrow as a novel model to study pathophysiology and drug resistance in multiple myeloma. Biomaterials 73, 70-84, doi:10.1016/j.biomaterials.2015.09.017 (2015)). A 2D-layer of human umbilical endothelial cells (HUVECs, ATCC® CRL-1730™) or primary human ECs grown on nanofibrous meshes will be used to recreate the endothelium. Patient BMMCs will be introduced to the tissue cultures to conduct cell-cell adhesion studies as described below.

7.2 O₂ Tension Control

The cell culture platform will be placed on a Nikon Ti-E inverted microscope equipped with an automated stage housed in an environmental chamber (In Vivo Scientific). The microscope incubator will be updated with an Oxystreamer gas controller (Warner Instruments), to independently control O₂ and CO₂ to within 0.1%. Culture medium will be supplied to the culture platform using syringe pumps (KDS230; KD Scientific) located outside of the incubator via low-density PE tubing. Because O₂ diffusivity in the tube material is high (4×10⁻¹² m²/s), the culture medium can be equilibrated to the incubator O₂ content in a short distance (e.g., 2 cm length from 20 to 4% at 1 μL/min). The medium will be sampled at the inlet and outlet locations of the culture platform, and O₂ concentrations will be measured using microelectrode-based sensors (OX-10, Unisense) with 10 μm probe size and 0.3 s response time.

7.3 Cell-Cell Adhesion

Time-lapse microscopy will be performed as previously described (Zhang, W., Lee, W. Y., Siegel, D. S., Tolias, P. & Zilberberg, J. Patient-Specific 3D Microfluidic Tissue Model for Multiple Myeloma. Tissue Eng Part C Methods, doi:10.1089/ten.TEC.2013.0490 (2014)) to visualize cell-cell adhesion steps in real-time and to quantify the percentage of adherent vs. non-adherent PMMCs. As summarized in Table 3, the cell cycle, proliferation and sternness of adherent vs. non-adherent PMMCs will be analyzed by flow cytometry. In brief, BMMCs will be introduced in the cell culture platform (FIG. 1), allowed to interact with the endosteal niche surface for 24 h and exposed to a flow rate of 100 μL/min to remove weakly adhered cells. Strongly adhered cells will be allowed to interact for another 24 h prior to analyses. Scanning electron microscopy (SEM) will be used to ascertain PMMC-OSB-OST adhesion with focus on possible connection of OSTs' dendritic processes to PMMCs. SEM samples will be prepared by embedding the tissue samples in PMMA resin, etching with phosphoric acid to remove mineralized ECM and highlight the morphology of dendritic processes and connection to PMMCs, dehydrating in sequential ethanol solutions with increasing concentrations from 50 to 100%, and coating with gold prior to SEM (Zeiss Auriga FIB-SEM). Tissue samples will be fixed in 4% paraformaldehyde for histological evaluation with hematoxylin and eosin (H&E) staining to quantify the distribution of PMMC populations that adhered to the 3D tissue surface.

TABLE 3
Readouts for Bone Core and Tissue Models

| | Bone | Tissue |
|---|---|---|
| Methods & Assays | Core | Models |
| Flow Cytometry - Dormant/Proliferative State | | ✓ |
| Cell Cycle (G0/G1, S, M phase) - P1 | | |
| PMMC proliferation - CFSE or CellTrace Violet | | |
| Sternness marker - Aldefluor assay | | |
| PMMC populations - CD138, CD38, CD56 | | |
| Immunohistochemistry | ✓ | ✓ |
| Bone cell morphology - H&E | | |
| Proliferating cells - Ki67 | | |
| PMMC populations - CD138, CD38, CD56 | | |
| Cell cycle - Cyclin D1/D2/D3 proteins | | |
| Hypoxia markers - PIM, HIF-1a | | |
| Intercellular EC junction marker - CD31 | | |
| Scanning Electron Microscopy | ✓ | ✓ |
| Morphological cell-cell connection | | |
| High Content Screening - Drug Evaluation | | ✓ |
| Live/dead staining using 7-AAD | | |
| PMMC populations - CD138, CD38, CD56 | | |
| PMMC proliferation - CFSE | | |
| Time-Lapsed Microscopy | | ✓ |
| Real-time imaging of cell-cell adhesion | | |

The effects of blocking N-cadherin and VLA4 integrin (which is expressed by MMCs and binds to VCAM-1 on the surface of BM stromal cells) will also be investigated as a function of O2 tension. An antibody against N-cadherin (GC4, Sigma) and its isotype control (Sigma) will be used to pretreat the tissues for 12 h prior to co-culture at concentrations of 0.1, 0.5 or 1 ug/mL. A VLA4 inhibitor (BIO01211, R&D systems) also will be used to pretreat PMMCs at 10, 50 or 100 pM. The treatments will be followed by time-lapse microscopy.

The adhesion of PMMC populations to the ECM and endothelium niche surfaces will be assessed microscopically, by flow cytometry and by immunohistochemistry to analyze the percentage, proliferative, cell cycle, and stemness state, of adherent vs. non-adherent PMMCs. Results will be compared to ascertain whether certain PMMC populations adhere strongly to the endosteal niche surface to become dormant under a critical O₂ tension level (e.g., 4%) and whether PMMCs at the ECM and endothelium niche surfaces become less dormant despite lower O2 tension levels (e.g., 1%).

PMMC populations isolated from bone core biopsies taken from 30 multiple myeloma (MM) patients will be cultured on endosteal, ECM and endothelium surfaces. The percentages of PMMC populations at endosteal, ECM, and endothelium locations will be identified and correlated to those of the matching PMMCs cultured in the respective niche surfaces (Table 4).

TABLE 4
Characterization of ex vivo Niche-specific Tissue
Models with 30 Patient Bone Core Samples

| | Bone Core | Tissue Models |
|---|---|---|
| Adhesion | ✓ | ✓ |
| Cell cycle, dormancy, proliferation | ✓ | ✓ |
| Drug resistance | | ✓ |

### Example 8. Dormant and Proliferative States and Drug Response of PMMCs Cultured in Endosteal, ECM and Endothelium Tissue Models as a Function of O₂ Tension

8.1 Design of Niche-Specific Tissue Models

The 2-well culture configuration (FIG. 1), along with the revised platform fabrication approach (FIGS. 5A, 5B and 8), will be used to construct the endosteal, ECM, and endothelium tissue niches. As shown in FIGS. 1 and 4C, the endosteal tissue niche model will consist of the 3D-OST tissue containing a 2D-OSB surface layer. The endothelium niche tissue will be constructed by growing a 2D-layer of human umbilical vein endothelial cells (HUVECs) on nanofibrous meshes. The ECM niche tissue model will be reconstructed by mixing patient BMMCs which include PMMCs and BM stromal cells and fibrin-based ECM as described above. The pore size of fibrin depends on the thickness and density of fibrils, which in turn, depend on the fibrinogen concentration in the plasma. For example, the calculated radius for fibrin for 8 mg/mL fibrinogen is estimated to be ˜7 μm (Chiu, C. L., Hecht, V., Duong, H., Wu, B. & Tawil, B. Permeability of three-dimensional fibrin constructs corresponds to fibrinogen and thrombin concentrations. BioResearch open access 1, 34-40, doi:10.1089/biores.2012.0211 (2012)). Based on this information, we will control the porosity of the ECM (5-8 μm) by changing the concentration of plasma (i.e., fibrinogen) in the mixture to ensure medium perfusion through the ECM-niche tissue structure.

8.2 O₂ Tension Control

The well plate-based culture device was placed inside a tri-gas incubator (Heracell 160i, ThermoFisher) equipped with an infrared sensor for precise oxygen control in the range of 1 to 20%, i.e., 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20%.

For the design of our tissue models, COMSOL Multiphysics® (COMSOL Inc.) simulations were used to assess the feasibility of precisely controlling O2 tension in our tissue models. By way of example, the following equations were used to analyze the development of O2 concentration gradients through the 3D-OST and 2D-OSB tissue regions due to metabolic consumption:

$${{{R}_{b} = - {D}_{b} \frac{{\partial }^{2} C}{\partial {x}^{2}} + {u}_{b} \frac{\partial C}{\partial x} for 3 D \text{-} OST \\ {R}_{b} = - {D}_{b} \frac{{\partial }^{2} C}{\partial {x}^{2}} + {u}_{b} \frac{\partial C}{\partial x} for 2 D \text{-} OST \\ - {D}_{o} \frac{\partial C}{\partial x} )}_{o} + {u}_{o} c = - {D}_{b} \frac{\partial C}{\partial x} )}_{b} + {u}_{b} c$$

$$at OSB \text{/} OST interface$$

where C is the concentration of O2 dissolved in the culture medium with subscripts “b” and “o” denoting the 3D-OST and 2D-OSB tissue compartments, respectively; R is the volumetric O2 consumption rate by cells; and u and D are the effective linear velocity of the culture medium and the effective diffusion coefficient of O2 which are normalized to the porosity (ε) of the specific compartment, respectively.

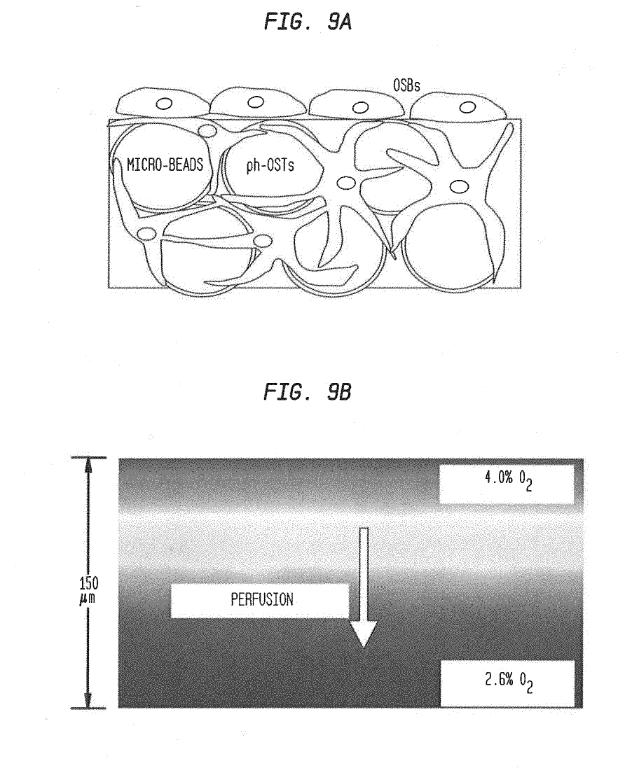

As summarized in Table 5, the physical and metabolic kinetic parameters were assumed to be constant through the thickness of each tissue compartment (i.e., R is independent of C) and estimated using our experimental data and literature values. Notably, R=nRi where n is the volumetric cell number density and Ri is the O2 consumption rate of a single cell and the normoxic values for Ri were used for the initial analysis due the lack of hypoxic data in the literature. The simulation results (FIG. 9B) suggest that, if the O2 tension of the culture medium entering the tissue is 4%, the O2 tension at the exit location of the tissue will be 2.6%. For the ECM niche tissue, the O2 consumption rate and cell number density of BMMCs are relatively low to cause any significant O2 tension gradient. Also, no significant O2 tension gradient exists through the 2D-EC layer due to its small thickness. Simulation results will be confirmed by measuring the inlet and outlet O2 concentrations of the culture medium using the method described in Example 7.2.

TABLE 5
Physical and Kinetic Parameters Used for O₂ Tension Gradient
Simulations

| | 3D-OST | 2D-OSB | 3D-BMMC | 2D-EC |
|---|---|---|---|---|
| n | 10⁵ | 2.5 × 10⁶ | 4 × 10³ | 2 × 10⁶ |
| R | 1.2 | 30 | 0.03 | 100 |

n = cells/mm³; R = pmol/mm³/s

8.3 Niche-Specific Dormant and Proliferative States of PMMCs

As summarized in Table 3, cell cycle, proliferation and sternness of PMMCs cultured in the endosteal, ECM, and endothelium niche models will be analyzed by flow cytometry and immunohistochemistry.

8.4 Niche-Specific Drug Response of PMMCs Cultured in Tissue Models

The response of PMMCs cultured in the endosteal, ECM, and endothelium niche models to drug treatments that target proliferation, proteasomal inhibition, adhesion, and hypoxia will be evaluated and compared. The drug evaluation studies will be performed with both human and murine versions of the endosteal and endothelium models. For the murine version, 5T33 murine MMCs will be cultured in the endosteal model constructed with murine MLO-A5 cells (FIG. 1) and the endothelium model prepared with murine 2F-2B (ATCC® CRL-2168™) ECs.

The tissue samples will be cultured at 1%, 4% or 20% O2, followed by seeding and culturing BMMCs for 48 h, prior to treatment with bortezomib (a proteasome inhibitor, 0-10 nM) for 1 h, melphalan (a chemotherapeutic agent that targets proliferation, 0-50 μM) for 1 h, or hypoxia pro-drug TH-302 (a 2-nitroimidazole prodrug, which exhibits hypoxia-selective cytotoxicity to 5T33MM cells, Sellckchem.com, 0-50 μM) for 24 h. The tissue samples also will be treated with an antibody against N-cadherin (GC4, Sigma) to directly disrupt adhesion of PMMCs to the tissue samples or its isotype control (Sigma) [0.1-1.5 ug/mL] for 12 h prior to seeding and culturing BMMCs for 24 h. Analyses will be conducted 24 h after treatment.

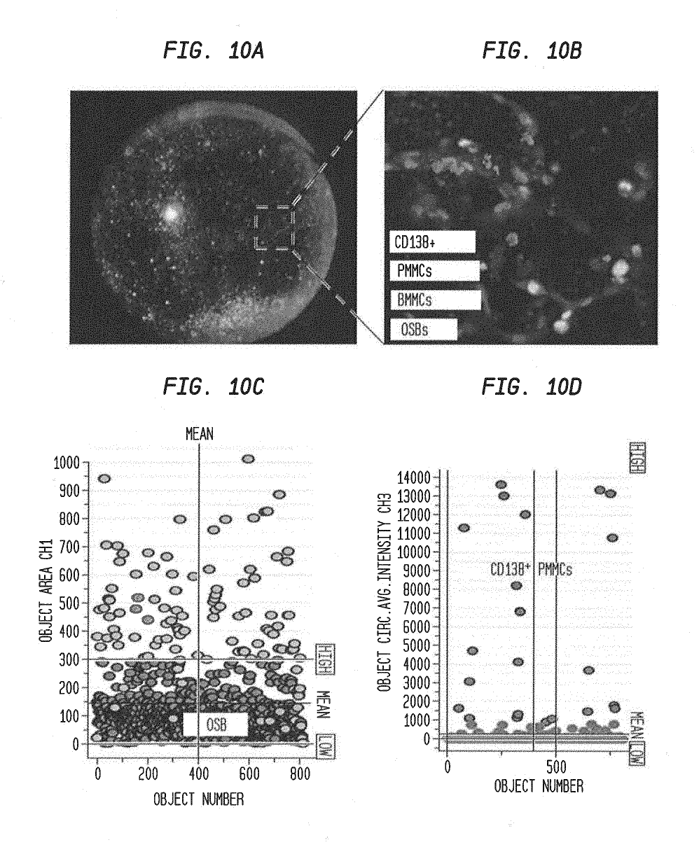

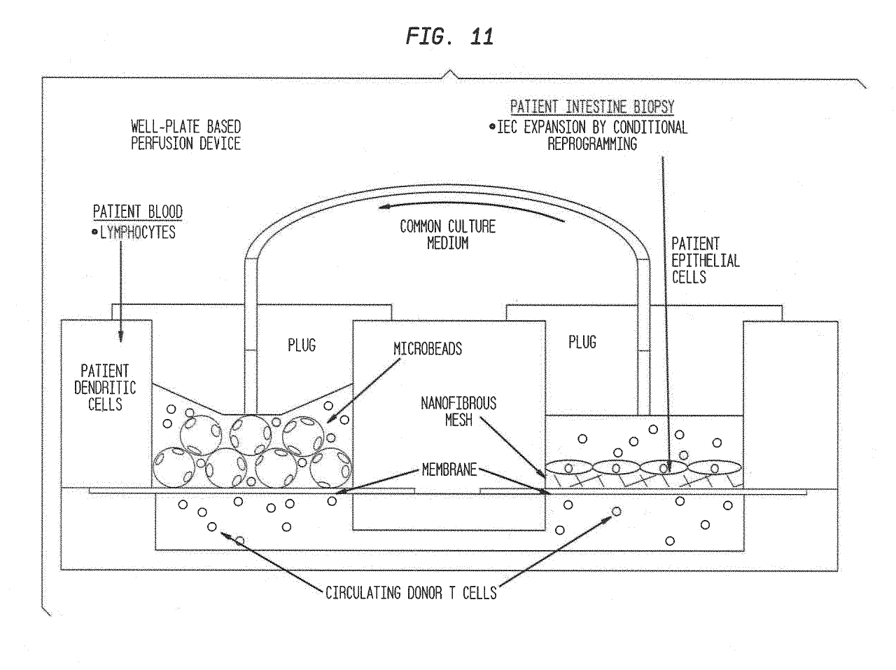

PMMCs cultured and treated in the tissue models, after 1-day post-treatment, will be stained for surface markers and cell viability for high-content screening (HCS) analyses. The Celllnsight™ CX5 HCS platform from ThermoFisher (with 5-filter capability 740/809, 650/694, 485/521, 386/440, 560/607) will be used to quantitate the viability of these PMMCs by counting live and dead cells. FIGS. 10A-10D show an example of data acquired and analyzed using HCS. The following stains will be further tested and optimized to follow multiple PMMC populations: CD38 VioBright FITC (496/522), CD138 Alexa Fluor 546 (556/573), and CD56 APC-eFluor® 780 (633/780). MM stem cell-like cells will be assessed with ALDEFLUOR™ Kit for aldehyde dehydrogenase (ALDH1, 490/525). Cell viability will be assessed using 7-AAD (546/647). BMMCs will be pre-labeled with CellTrace violet (405/450) in order to easily differentiate OSB from BMMCs. IC50 values will be obtained as a function of dormancy/proliferation (i.e., CFSEhigh vs. CFSElow or CellTracehigh vs. CellTracelow), cell cycle and sternness (Table 3).

Combined with the dormant and proliferative assay results described above, the IC50 results from the human version of the niche-specific tissue models will be compared and used to ascertain whether cell adhesion mediated drug resistance (CAM-DR) of dormant PMMC populations can be mediated by the endosteal niche under a critical O2 tension level (e.g., 4%) and whether certain PMMC populations may become less dormant and exhibit higher sensitivity to drugs that target proliferation at the ECM and endothelium niches, despite lower O2 tension levels (e.g., 1%).

The IC50 results from the murine version of the niche-specific tissue models will be compared to the following in vivo observations reported in the literature (Mrozik, K. M. et al. Therapeutic targeting of N-cadherin is an effective treatment for multiple myeloma. British journal of haematology 171, 387-399, doi:10.1111/bjh.13596 (2015); Lawson, M. A. et al. Osteoclasts control reactivation of dormant myeloma cells by remodelling the endosteal niche. Nature communications 6, 8983, doi:10.1038/ncomms9983 (2015); Hu, J. et al. Targeting the multiple myeloma hypoxic niche with TH-302, a hypoxia-activated prodrug. Blood 116, 1524-1527, doi:10.1182/blood-2010-02-269126 (2010); Hu, J. et al. Synergistic induction of apoptosis in multiple myeloma cells by bortezomib and hypoxia-activated prodrug TH-302, in vivo and in vitro. Molecular cancer therapeutics 12, 1763-1773, doi:10.1158/1535-7163.MCT-13-0123 (2013)).

8.5 Tissue Model Characterization

As summarized in Table 4, cell cycle, hypoxic state and proliferation of PMMC populations isolated form bone core biopsies taken from 30 multiple myeloma (MM) patients will be analysed and compared to the matching PMMCs cultured in the respective niche tissue models.

### Example 9. Multiwell Plate-Based Microfluidic Perfusion Culture Device for Ex Vivo Modeling of Persistent T Lymphocyte Stimulation Events at Lymph Node and Tissue Levels In Vivo

### Example 9.1 Generating Conditionally Reprogrammed IECs from Murine Samples

Successful isolation of primary murine m-IECs (small intestine) from adult 6-8 week old mice was performed as detailed by Evans et al. (“The development of a method for the preparation of rat intestinal epithelial cell primary cultures,” J. Cell Sci. 101 (Pt 1), 219-231 (1992)), with modifications from techniques in Zilberberg's lab.

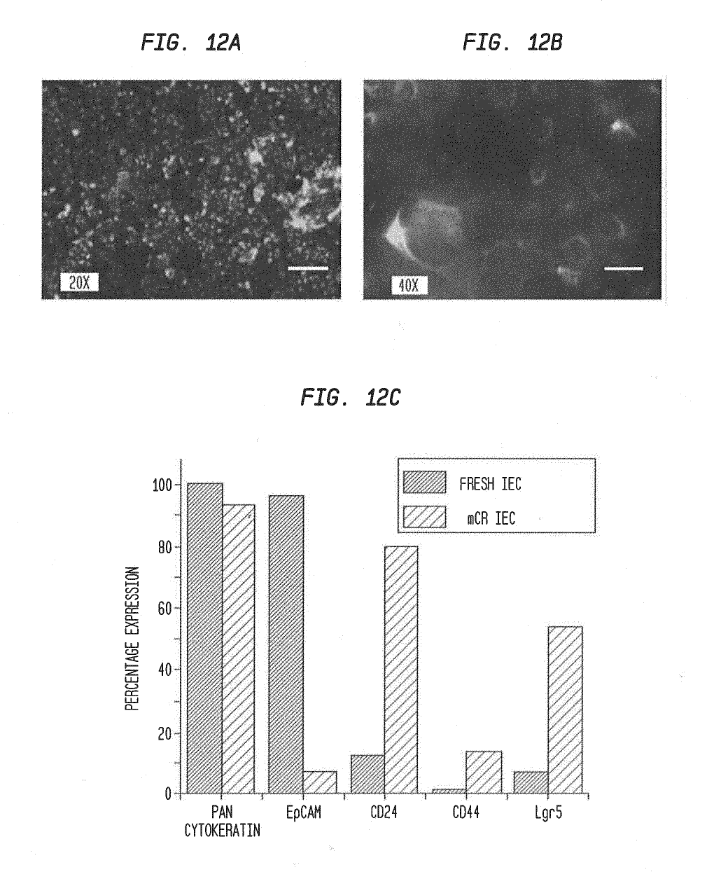

Expansion of m-IECs was performed using CR (Palechor-Ceron, N. et al., “Radiation induces diffusible feeder cell factor(s) that cooperate with ROCK inhibitor to conditionally reprogram and immortalize epithelial cells,” Am. J. Pathol. 183, 1862-1870, doi:10.1016/j.ajpath.2013.08.009 (2013); Liu, X. et al., “ROCK inhibitor and feeder cells induce the conditional reprogramming of epithelial cells,” Am. J. Pathol 180, 599-607, doi: 10.1016/j.ajpath.2011.10.036 (2012); Suprynowicz, F. A. et al., “Conditionally reprogrammed cells represent a stem-like state of adult epithelial cells,” Proc. Nat. Acad. Sci. USA 109, 20035-20040, doi: 10.1073/pnas. 1213241109 (2012)). Freshly isolated m-IECs and m-CRIEC were >98% positive pan cytokeratin and epithelial cell adhesion molecule (EpCAM) (ref. 14, 40) positive, confirming the purity of our cultures (see FIGS. 12A and B). Gene expression analysis also corroborated that these cells significantly expressed cytokeratin 8 (KRT8; a specific marker for IECs) with low expression of cytokeratin 15 (KRT15; a marker for skin epithelium (Zhan, Q. et al., “Cytokeratin 15-positive basal epithelial cells targeted in graft-versus-host disease express a constitutive antiapoptotic phenotype, J. Invest. Dermatol. 127, 106-115, doi:10.1038/sj.jid.5700583 (2007); Whitaker-Menezes, D., et al, “An epithelial target site in experimental graft-versus-host disease and cytokine-mediated cytotoxicity is defined by cytokeratin 15 expression,” Biology of Blood and Marrow Transplantation: 9, 559-570 (2003).) (data not shown).

Upon CR expansion with conditioned medium (Palechor-Ceron, N. et al., “Radiation induces diffusible feeder cell factor(s) that cooperate with ROCK inhibitor to conditionally reprogram and immortalize epithelial cells,” Am. J. Pathol. 183, 1862-1870, doi:10.1016/j.ajpath.2013.08.009 (2013).), mCRIECs acquired a stem-like phenotype (increased CD24 and Lgr5 in the case of IECs) as reported to be the case with other primary epithelial cells (Saenz, F. R. et al., “Conditionally reprogrammed normal and transformed mouse mammary epithelial cells display a progenitor-cell-like phenotype,” PloS One 9, e97666, doi:10.1371/joumal.pone.0097666 (2014); (2014); Suprynowicz, F. A. et al., “Conditionally reprogrammed cells represent a stem-like state of adult epithelial cells,” Proc. Nat. Acad. Sci. USA 109, 20035-20040, doi: 10.1073/pnas. 1213241109 (2012)) undergoing CR (FIG. 2B). These results with m-IECs suggest that CRIECs can be produced while preserving characteristic IEC functions.

### Example 9.2 Collecting Human Biospecimens

Donor and recipient peripheral blood lymphocytes (PBL) and allogeneic-BMT recipient GI biopsies (e.g., taken from colonoscopies) will be collected in accordance with the IRB approved protocol. GI specimens will be collected at the onset of GVHD if GI biopsies already are being performed. Transplant patients, undergoing a gut biopsy as part of their standard of care, will be asked to donate two extra cores of approximately 3 mm in size. Overly inflamed tissue samples will not be used in this study.

Blood collection will be performed prior to transplant to ensure the collection of viable cells (four 8.5 mL yellow top tubes per individual, containing 106 cells/mL, of which half are T cells). Donor blood will be used to isolate T cells for killing assays, and patient blood will be utilized to develop DCs as specified below. PBL will be obtained by centrifugation of blood samples over Ficoll-Paque-Plus (Friedman, T. M. et al., “Overlap between in vitro donor antihost and in vivo posttransplantation TCR Vbeta use: a new paradigm for designer allogeneic blood and marrow transplantation,” Blood 112, 3517-3525, doi: 10.1182/blood-2008-03-145391 (2008)) and cryopreserved for later use in killing assays. Upon collection, tissue samples will be place in PBS at 4° C.

### Example 9.3 Generating h-CRIECs from Patients and Murine Models

h-CRIECs will be prepared following procedures developed for the generation of m-CRIECs (see, e.g., Saenz, F. R. et al. Conditionally reprogrammed normal and transformed mouse mammary epithelial cells display a progenitor-cell-like phenotype. PloS One 9, e97666, doi:10.1371/joumal.pone.0097666; (2014); Palechor-Ceron, N. et al. Radiation induces diffusible feeder cell factor(s) that cooperate with ROCK inhibitor to conditionally reprogram and immortalize epithelial cells. Am. J. Pathol. 183, 1862-1870, doi:10.1016/j.ajpath.2013.08.009 (2013); Liu, X. et al. ROCK inhibitor and feeder cells induce the conditional reprogramming of epithelial cells, Am. J. Pathol. 180, 599-607, doi: 10.1016/j.ajpath.2011.10.036 (2012)). All CRC will be cryopreserved until use.

### Example 9.4 Validating the Utility of the In Vitro GVHD (iGVHD) Platform Using Clinically Relevant Murine Models of Allo-BMT

TABLE 6
Murine models and experiments for biological
validation of the iGVHD concept and utility.

| Murine BMT model | Expected Outcome |
|---|---|
| miHA model | Using the miHA model B6→BALB.B, with known GVHD |
| B6→BALB.B | potential (Zilberberg, J., McElhaugh, D., Gichuru, L. N., |
| | Korngold, R. & Friedman, T. M. Inter-strain tissue- |
| | infiltrating T cell responses to minor histocompatibility |
| | antigens involved in graft-versus-host disease as determined by |
| | Vbeta spectratype analysis, J. Immunol. 180, 5352-5359 |
| | (2008)), the percentage of killed IEC to aid the development of |
| | an empirical pathological index (PIdx). This PIdx will be put |
| | in practice to assess donor-patient pair reactivity in clinical |
| | samples. |
| Negative Control | B6→CXB-7 will be used as a negative control to identify the |
| B6→CXB-7 | lower limit of the killing assay, i.e., to help determine what |
| | degree of IEC apoptosis can be expected in the absence of in |
| | vivo GVHD-induced lethality.. No substantial damage of |
| | CXB-7 IEC in this nonlethal miHA model, which has a subset |
| | of the miHA expressed by the BALB-B strain, is expected. |
| | Some apopotosis may occur, since some cachexia can be |
| | observed in recipient mice. (Korngold, R. & Wettstein, P. J. |
| | Immunodominance in the graft-vs-host disease T cell response |
| | to minor histocompatibility antigens. J. Immunol. 145, 4079- |
| | 4088 (1990)). |
| Haploidentical transplant | To recapitulate the clinical scenario where haploidentical |
| model, with three different | transplant recipients undergo cyclophosphamide treatment on |
| potential donors and | day 3 post-BMT to eliminate highly alloreactive MHC_specific |
| syngeneic negative control: | T cells and thereby lessen the severity of GVHD (Kanda, J., |
| B6→B6D2F1; BALB.B→ | Chao, N. J. & Rizzieri, D. A. Haploidentical transplantation for |
| B6D2F1; C3H.SW→ | leukemia, Cur. Oncol. Reports 12, 292-301, doi: 10.1007/sl |
| B6D2F1; B6D2F1 −> | 1912-010-0113-4 (2010); Luznik, L., O'Donnell, P. V. & |
| B6D2F1. | Fuchs, E. J. Post-transplantation cyclophosphamide for |
| | tolerance induction in HLA-haploidentical bone marrow |
| | transplantation. Sem. Oncol. 39, 683-693, doi: |
| | 10.1053/j.seminoncol.2012.09.005 (2012)), cultures will also |
| | be treated with an analog of cyclophosphamide as described |
| | (Kanakry, C. G. et al. Aldehyde dehydrogenase expression |
| | drives human regulatory T cell resistance to posttransplantation |
| | cyclophosphamide. Sci. Translat. Med. 5, 21 Ira 157, doi: |
| | 10.1126/scitranslmed.3006960 (2013)). This would leave the T |
| | cell responses to be directed mostly to miHA differences. |
| | The killing assays will thus be utilized here to predict the best |
| | donor for the B6D2F1 recipient, i.e., the donor that will incur |
| | the least degree of pathological damage (as determined by the |
| | PIdx). It is expected that the donor with the lowest PIdx score |
| | will likely induce less GVHD in vivo. This will be correlated |
| | with in vivo BMT GVHD experiments using methodology that |
| | has been described (Fanning, S. L. et al Unraveling graft- |
| | versus-host disease and graft-versus-leukemia responses |
| | using TCR Vbeta spectratype analysis in a murine bone |
| | marrow transplantation model. J. Immunol. 190, 447-457, doi: |
| | 10.4049/jimmunol. 1201641 (2013); Zilberberg, J., et al., Inter- |
| | strain tissue-infiltrating T cell responses to minor |
| | histocompatibility antigens involved in graft-versus-host |
| | disease as determined by Vbeta spectratype analysis. J. |
| | Immunol 180, 5352-5359 (2008)). The syngeneic negative |
| | control using B6D2F1 donor cells will provide the baseline for |
| | the PIdx. |

Preliminary Results Suitability of cRPMI:

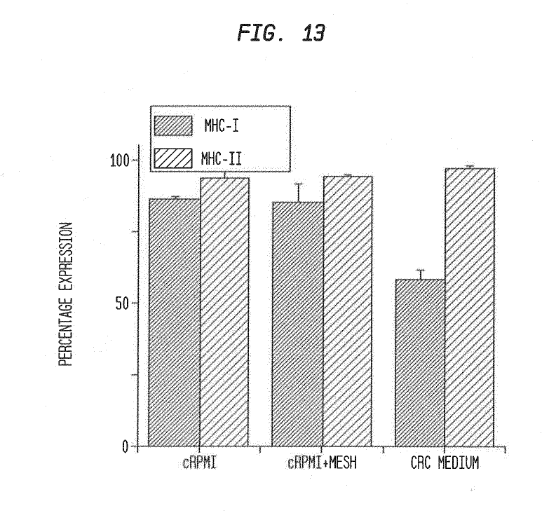

Since CRC medium additives (e.g., ROCK kinase inhibitor) can ameliorate GVHD (Iyengar, S., Zhan, C., Lu, J., Korngold, R. & Schwartz, D. H. Treatment with a Rho Kinase Inhibitor Improves Survival from Graft-Versus-Host Disease in Mice after MHC-Haploidentical Hematopoietic Cell Transplantation. Biol. Blood Marrow Transplant., doi:10.1016/j.bbmt.2014.04.029 (2014)) and therefore should not be used for co-culture of m-CRIECs and T cells, the CRC medium was replaced with complete RPMI-1640 medium (cRPMI, RPMI medium supplemented with 10% FBS and 5% L-glutamine), which is conventionally used to culture T cells (Friedman, T. M. et al. Overlap between in vitro donor antihost and in vivo posttransplantation TCR Vbeta use: a new paradigm for designer allogeneic blood and marrow transplantation. Blood 112, 3517-3525, doi: 10.1182/blood-2008-03-145391 (2008)) (FIG. 12B). m-CRIEC's upregulation of surface expression of major histocompatibility complexes I and II (MHCI and MHC II; the murine equivalent of human HLA) (FIG. 13). The increased expression of these molecules serves as a catalytic step without which T cells cannot recognize miHA or any antigen on the surface of host cells (Korngold, R. & Sprent, J. Graft-versus-host disease in experimental allogeneic bone marrow transplantation. Proc. Soc. Exp. Biol. Med. Soc. Exp. Biol. Med. 197, 12-18 (1991); Korngold, R. & Sprent, J. Surface markers of T cells causing lethal graft-vs-host disease to class I vs class II H-2 differences; J. Immunol. 135, 3004-3010 (1985)). The results indicate that, while other culture media partially hindered the upregulation of these molecules, cRPMI permitted maximum expression of MHC-I and MHC-II after 72 h of cytokine exposure.

### Example 9.5 Feasibility of Using Nanofibrous Mesh in Maintaining the Long-Term Functionality of CRIECS

Basement membrane (BMa)-like fibrous meshes with random fiber organization were prepared by electrospinning (Yang, X., Ogbolu, K. R. & Wang, H. Multifunctional Nanofibrous Scaffold for Tissue Engineering. J. Exp. Nanoscience 3, 329-345 (2008)). To obtain stable and strong nanofibers, slow degradable, biocompatible polycaprolactone (PCL) was used as the fiber matrix phase in which Type IV collagen (representing ECM molecules) was dispersed.

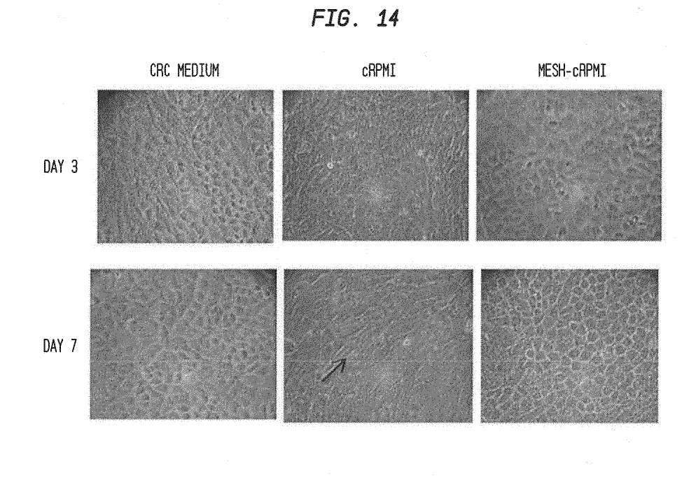

As shown in FIG. 14, the combination of nanofibrous mesh and cRPMI culture medium enabled the maintenance of viable and morphologically sound CRIECs, even after 7 days in the absence of CR medium.

As shown in Table 7 flow cytometric analysis of annexin V+/propidium iodide (PI)+staining showed that mCRIEC viability decreased at an E:T ratio of 5:1 as determined by increased apoptotic cells (% Annexin V+). At a ratio of 10:1, 64.8% of m-CRIEC were dead (double+) by day 6. PIdx=3.83.

TABLE 7

| E:T ratio | % Annexin V+ | % AnV+/PI+ |
|---|---|---|
| m-CRIEC | 14.5 | 11.8 |
| 5:1 | 71.3 | 19.3 |
| 10:1 | 67.4 | 64.8 |

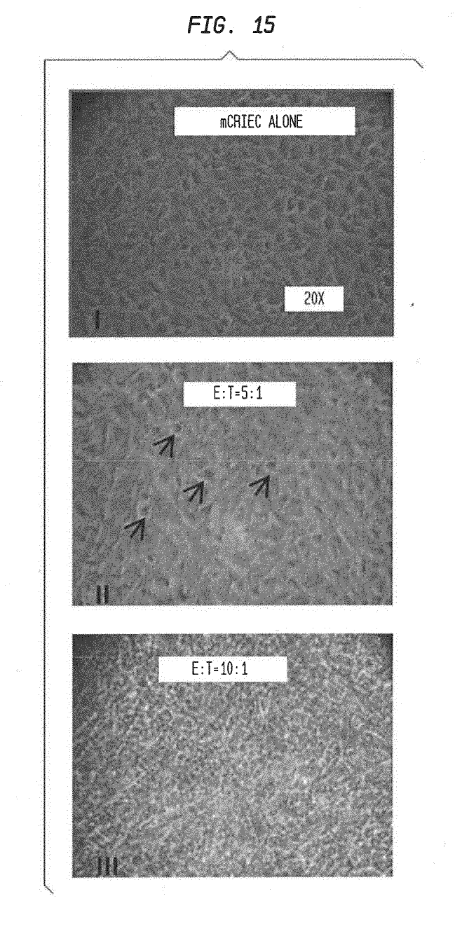

Also, the above culture conditions were sufficient to enable anti-allogeneic T cell responses capable of inducing quantifiable reaction to m-CRIECs in an MHC-mismatched setting (FIG. 15, and Table 6). The pathological index (PIdx) was calculated to be 3.83.

### Example 9.6

### Experiments to Establish the Pathological Index (PIdx) to Quantify T Cell Induced IEC Damage and Killing:

The predictive capability of co-culture killing assays and later iGVHD will be compared to known in vivo outcomes from well-established murine models of BMT (Table 6). These models represent different degrees of alloantigenic barriers and hence distinct clinical scenarios:

the miHA-disparate C57BL6/J (B6)→C.B10-H2b/LiMedJ (BALB.B) and B6→CXB-7/By (CXB-7) models (see Zilberberg, J. et al, “Inter-strain tissue-infiltrating T cell responses to minor histocompatibility antigens involved in graft-versus-host disease as determined by Vbeta spectratype analysis,” J. Immunol. 180: 5352-59 (2008); Korngold, R. & Wettstein, P. J. “Immunodominance in the graft vs host disease T cell response to minor histocompatibility antigens,” J. Immunol. 145: 4079-4088 (1990); Jones, S. C. et al, “Specific donor Vbeta-associated CD4 T-cell responses correlate with severe acute graft versus host disease directed to multiple minor histocompatibility antigens. Biol. Blood Marrow Transplant. 10: 91-105, doi: 10.1016/j.bbmt.2003.10.002 (2004); Jones et al, “Importance of minor histocompatibility antigen expression by nonhematopoietic tissues in a CD4+ T cell-mediated graft-versus-host disease model,” J. Clin. Invest. 112: 1880-86, doi: 10.1172/JC119427 (2003); Friedman, T. M., et al, “Vbeta spectratype analysis reveals heterogeneity of CD4+ T cell responses to minor histocompatibility antigens involved in graft-versus-host disease: correlations with epithelial tissue infiltrate,” Biol. Blood Marrow Transplant. 7: 2-13, doi: 10.1053/bbmt.2001.v7.pm11215694 (2001); Friedman, T. M. et al, “Repertoire analysis of CD8+ T cell responses to minor histocompatibility antigens involved in graft-versus-host disease, J. Immunol 161: 41-48 (1998)), where both donor and recipients are MHC (H2b—matched); and

the haploidentical-MHC model (see Zilberberg, J. et al, “Inter-strain tissue-infiltrating T cell responses to minor histocompatibility antigens involved in graft-verus-host disease as determined by Vbeta spectratype analysis,” J. Immunol. 180: 5352-59 (2008); Korngold, R. & Wettsstein, P. J. “immunodominance in the graft vs host disease T cell response to mino histocompatibility antigens,” J. Immunol. 145: 4079-4088 (1990); Jones, S. C. et al, “Specific donor Vbeta-associated CD4 T-cell responses correlate with severe acute graft versus host disease directed to multiple minor histocompatibility antigens. Biol. Blood Marrow Transplant. 10: 91-105, doi: 10.1016/j.bbmt.2003.10.002 (2004); Jones et al, “Importance of minor histocompatibility antigen expression by nonhematopoietic tissues in a CD4+ T cell-mediated graft-versus-host disease model,” J. Clin. Invest. 112: 1880-86, doi: 10.1172/JC119427 (2003); Friedman, T. M., et al, “Vbeta spectratype analysis reveals heterogeneity of CD4+ T cell responses to minor histocompatibility antigens involved in graft-versus-host disease: correlations with epithelial tissue infiltrate,” Biol. Blood Marrow Transplant. 7: 2-13, doi: 10.1053/bbmt.2001.v7.pm11215694 (2001); Friedman, T. M. et al, “Repertoire analysis of CD8+ T cell responses to minor histocompatibility antigens involved in graft-versus-host disease, J. Immunol 161: 41-48 (1998)) B6→(B6×DBA/2)F1 [B6D2F1(H2b/d)] (Patterson, A. E. and Korngold, R., “Infusion of select leukemia-reactive TCR Vbeta+ T cells provides graft-versus-leukemia responses with minimization of graft-versus-host disease following murine hematopoietic stem cell transplantation,” Biol. Blood Marrow Transplant. 7: 187-196 (2001)). In brief, m-IECs from small and large intestine can be isolated from recipient strains and expanded using CR technology. The m-CRIECs can be cryopreserved for later use in co-culture experiments. The m-CRIECs can be cultured on nanofibrous matrices in the presence of complete RPMI supplemented with TNF-α and IFN-γ to induce upregulation of MHC-1 and MHC-II molecules.

In brief, for each of the experimental murine models proposed in Table 6, m-IECs (from small and large intestine) will be isolated from recipient strains and expanded using CR technology. The m-CRIECs will be cryopreserved for later use in co-culture experiments. The m-CRIECs will be cultured on nanofibrous matrices in the presence of cRPMI supplemented with TNFα and IFNγ to induce upregulation of MHC-1 and MHC-II molecules; an indispensable state to generate tissue-directed alloresponses (FIG. 13).

Although TNFα is best known for its inflammatory effects, it also can induce upregulation of programmed death ligand 1 (PDL-1) on the surface of cells, which acts as an immunological checkpoint and can shut down effector T cells. Preliminary data (not shown) indicates that epithelial cells upregulate PDL-1 under inflammatory conditions (Wu, Y. Y. et al Increased programmed death-ligand-1 expression in human gastric epithelial cells in Helicobacter pylori infection. Clin. Exp. Immunol. 161, 551-559, doi: 10.1111/j. 1365-2249.2010.04217.x (2010)), and thus TNFα can play an important regulatory role in allogeneic transplantation (Alderson, K. L. et al Regulatory and conventional CD4+ T cells show differential effects correlating with PD-1 and B7-H1 expression after immunotherapy. J. Immunol. 180, 2981-2988 (2008); Tanaka, K. et al PDL1 is required for peripheral transplantation tolerance and protection from chronic allograft rejection. J. Immunol. 179, 5204-5210 (2007); Saha, A. et al Host programmed death ligand 1 is dominant over programmed death ligand 2 expression in regulating graft-versus-host disease lethality. Blood 122, 3062-3073, doi: 10.1182/blood-2013-05-500801 (2013)). A PDL-1 blocker (e.g., MPDL3280A, Genentech), will be introduced in order to ensure that T cell reactivity is not negatively modulated through this pathway.

Likewise, to better recapitulate tissue-induced damage by preconditioning regimens (Ferrara, J. L., Levine, J. E., Reddy, P. & Holler, E. Graft-versus-host disease. Lancet 373, 1550-1561, doi: 10.1016/SO 140-6736(09)60237-3 (2009)), IEC can be treated with the same chemotherapeutic agents that patients typically receive prior to transplant. This may induce the expression of MHC-I and MHC-II on the IEC, priming the T cells for a more robust response.

### Mixed Lymphocyte Culture

To mimic the early activation/proliferation stage of T cells in the described in vitro system, a mixed lymphocyte culture (MLC) will be used. (Fanning, S. L. et al Unraveling graft-versus-host disease and graft-versus-leukemia responses using TCR Vbeta spectratype analysis in a murine bone marrow transplantation model. J. Immunol. 190, 447-457, doi: 10.4049/jimmunol. 1201641 (2013); Friedman, T. M. et al. Overlap between in vitro donor antihost and in vivo posttransplantation TCR Vbeta use: a new paradigm for designer allogeneic blood and marrow transplantation. Blood 112, 3517-3525, doi: 10.1182/blood-2008-03-145391 (2008)). In brief, donor T cells (i.e., responders; R) will be cultured with irradiated (30 Gy) recipient lymphocytes (i.e., stimulators; S) at a 1:2 R:S ratio. For human MLC, enriched PBL from the patients will be used to stimulate responding T cells from their donors. Natural killer cells will be depleted from donor T cells to diminish non-specific target cell killing by this subpopulation of lymphocytes. After 9 days, human MLC responders will be harvested and re-stimulated for another 8 days as before, with the addition of 20 U/ml of rIL-2.

MLC will be carried out in the antigen presenting cells (APC) culture chamber, as part of the iGVHD platform, to facilitate activation, expansion and concentration of alloreactive T cells. Dendritic cells, as opposed to bulk lymphocytes, will be used in iGVHD, with a T cell-DC (R:S) ratio of 5:1. Activated T cells from MLC will then be placed in CRIEC on nanofibers to monitor for epithelial cell death. Killing assays with specimens from murine models of allo-BMT (Table 1), where the GVHD response has been characterized in vivo, will be used in order to designate an empirical PIdx to quantitate the response.

### Objective Cell Death Analysis Methods

A panel of objective cell death analysis methods (e.g., Annexin V/PI staining) using flow cytometry and in situ detection of cleaved caspase-3 using immunofluorescence will be utilized to determine cell death. The percentage of dead cells is calculated as [% of Annexin V+/PI+ cells in co-cultures−% of Annexin V+/PI+ cells in IEC alone cultures] (flow cytometric measurement). CD3 staining also will be performed to identify adherent T cells contributing to the response.

Caspase-3 staining also will be conducted in the well plate (and later in the microfluidic chambers to determine cell death as [fluorescence intensity in co-culture−fluorescence intensity in IEC alone cultures]/[fluorescence intensity of DAPI staining, as an indicator of the number of nucleated cells in the cultures].

Cell death will be evaluated at three or more (if determined to be necessary) T cell-IEC ratios (i.e., the effector: target, or E:T ratio). According to some embodiments, the E:T ratio is 30, 10, or 3. (See Choksi, S. et al, “A cD8 DE loop peptide analog prevents graft versus host disease in a multiple minor histocompatibility antigen-mismatched bone marrow transplantation model,” Biol. Blood Marrow Transplant. 10: 669-680, doi: 10.1016/j.bbmt.2004.06.005 (2004)).

The PIdx will be determined as the slope of the curve of percentage of dead cells vs. E:T ratios, where a steeper slope indicates a higher risk for developing GVHD. The PIdx will be determined at 4 different time points (day 3, day 7, day 14 and day 21 post co-culture) in order to maximize the opportunity to observe a response while ensuring that faster reactions do not reach plateau before obtaining a quantifiable PIdx, and that slow-to-develop GVHD responses also can be captured.

Statistical considerations. Continuous random variables (i.e., flow cytometric data, in situ staining/caspase-3 readout, PIdx) will be summarized as mean (standard deviation) or median (interquartile range) depending on whether or not they are normally distributed. Categorical random variables (i.e., GVHD grading) will be presented as count (percentage). Comparison of continuous random variables between groups (i.e., comparing different murine allo-BMT models) will be performed using two-sided Student's t-test or 2-sided Wilcoxon rank sum test, analysis of variance (ANOVA), Kruskal-Wallis, as appropriate. Categorical variables will be compared using Fisher's exact test or Pearson's Chi-square test, as appropriate. Median survival of transplanted mice will be estimated by the Kaplan-Meier method. Any p<0.05 will be considered statistically significant. For reproducibility of PIdx and staining methods, repeated (test-retest) measurements of PIdx will be compared using two-sided paired t-tests or Wilcoxon signed rank test. Correlation of the replicate PIdx measurements will be examined using Pearson correlation coefficient or Spearman correlation coefficients. Reliability of the PIdx will be evaluated using intra-class correlation coefficient, coefficient of variation. To examine the effect of culture time on PIdx, a mixed model repeated measures analysis will be conducted with PIdx at different time points. The Bland-Altman plot will be used to assess agreement between flow cytometry and in situ staining.

### Example 9.7 Use of the Multiwell Plate-Based Microfluidic Perfusion Culture Device to Mimic Interactions of Circulating Murine T Cells (m-T Cells) with m-CRIECs and Murine Dendritic Cells (m-DCs)

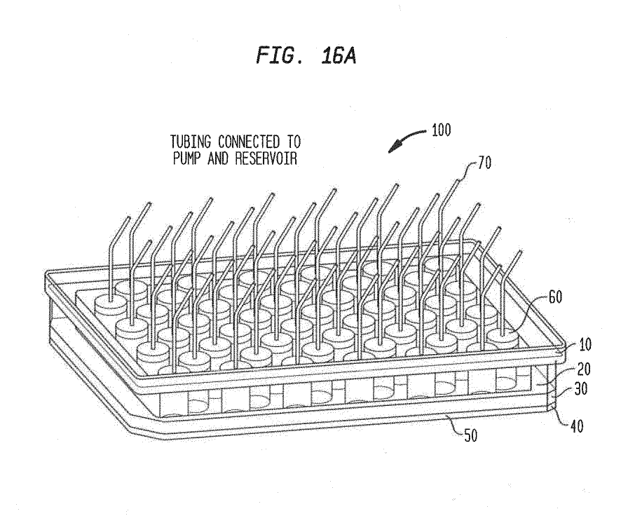

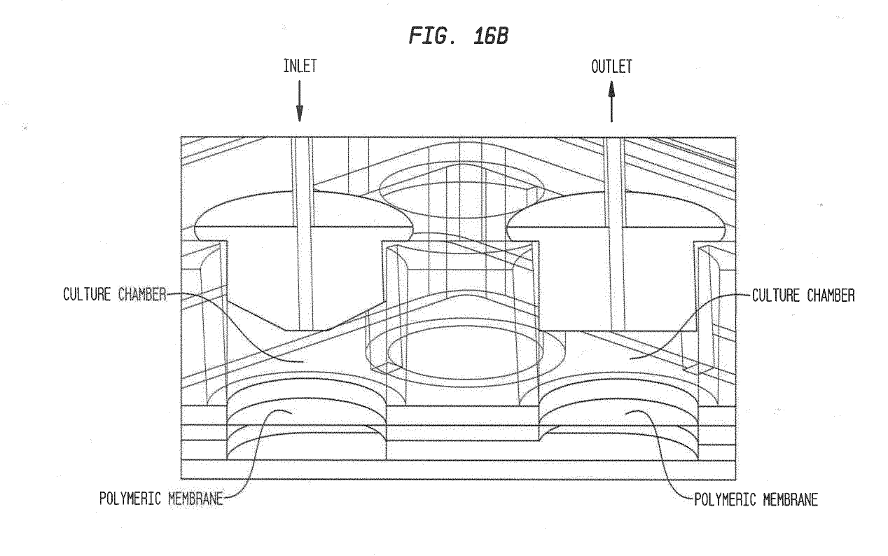

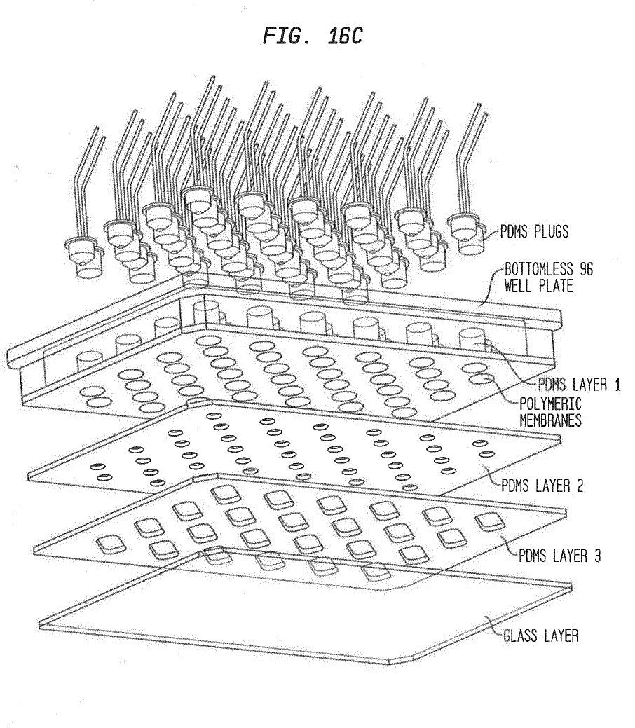

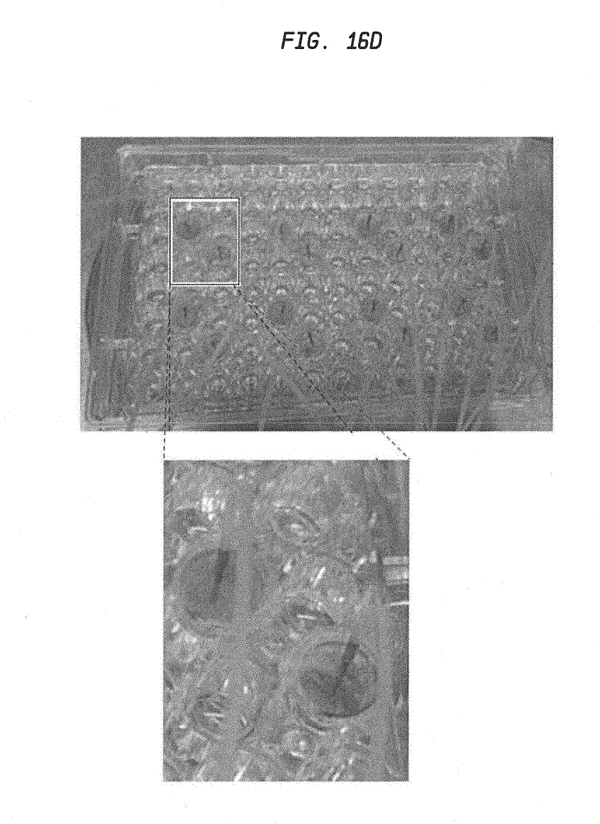

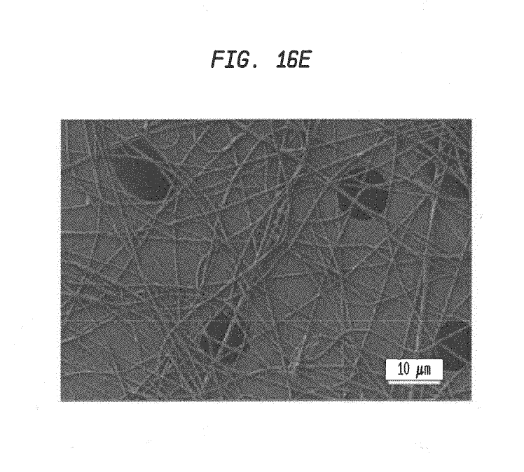

Our current prototype device (FIGS. 16A, 16B, 16C, 16D and 16E) was used to mimic interactions of circulating murine T cells (m-T cells) with m-CRIECs and murine dendritic cells (m-DCs) as illustrated in FIG. 1. One practical design feature of the device is the use of removable polydimethylsiloxane (PDMS) plugs at the top of the culture chambers to: (1) allow the convenient placement of cells and biomaterials into the culture chambers at various time points during culture; (2) externally interconnect culture the m-CRIEC and m-DC culture chambers using polyethylene tubing and a peristaltic pump (Model 78023-02, ISMATEC); and (3) recirculate m-T cells suspended in the RPMI common culture medium. The device uses transparent polycarbonate (PC) membranes (TCTP02500, Millipore) to: (1) anchor tissue cells and biomaterials, and (2) provide optical access through the bottom of the chambers for cell characterization with plate readers.

As shown in FIG. 16C, the prototype device was assembled with: (1) a commercial polystyrene (PS) bottomless 96-well plate (Model 655-000, Greiner Bio-One); 2) three micropatterned PDMS layers made by soft lithography, and (3) one blank glass layer. The PDMS layers were used to: (1) provide a microfluidic channel of 200 μm thick and 5 mm wide for use as an internal fluidic passage between the chambers, and (2) anchor the placement of the PC membranes within the culture chambers. The bottom of the device was sealed with the 1.2 mm-thick glass layer for use with plate readers. These parts were bonded using oxygen plasma treatments.

For our preliminary study, the average pore of the PC membranes was selected to be 10 μM in order to provide sufficient opening for m-T cells to go through, since the average diameter of m-T cells is about 5 μm. For the m-CRIEC culture chamber, the membrane was coated with electrospun PCT/collagen nanofibrous meshes (FIG. 16E).

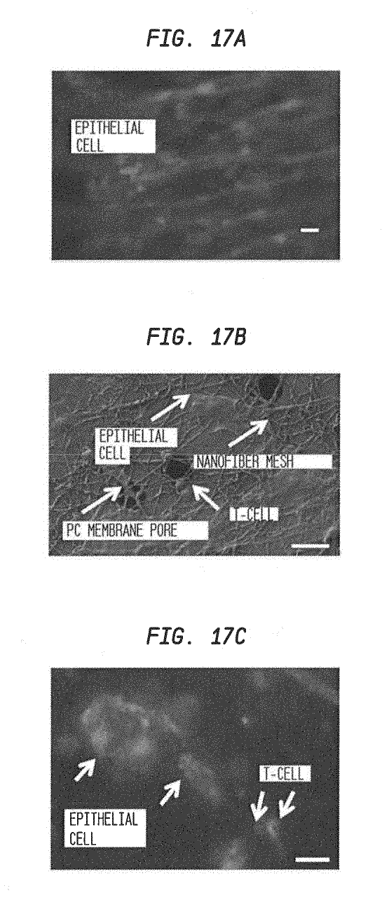

The device was used to culture m-CRIECs prepared from the small intestine of a B6 mouse. The cells develop a confluent layer while maintaining their viability up to 7 days (FIG. 17A).

After the m-CRIEC culture was established, eGFP m-T cells harvested from an eGFP transgenic B6 mouse were suspended in the culture medium (106 cells total) and introduced and circulated through the device. The SEM and fluorescence images in FIGS. 17B and C show that T cells were able to travel through the PC membrane and the nanofibrous meshes and interact with m-CRIECs through physical contact. Accordingly, these results show that the device can be used to promote physical interactions between m-CRIECs and circulating m-T cells. The a biomaterial that has been commonly used for 3D 1EC/organoid cultures (Sato, T. et al. Long-term expansion of epithelial organoids from human colon, adenoma, adenocarcinoma, and Barrett's epithelium. Gastroenterol. 141, 1762-1772, doi: 10.1053/j.gastro.2011.07.050 (2011)).

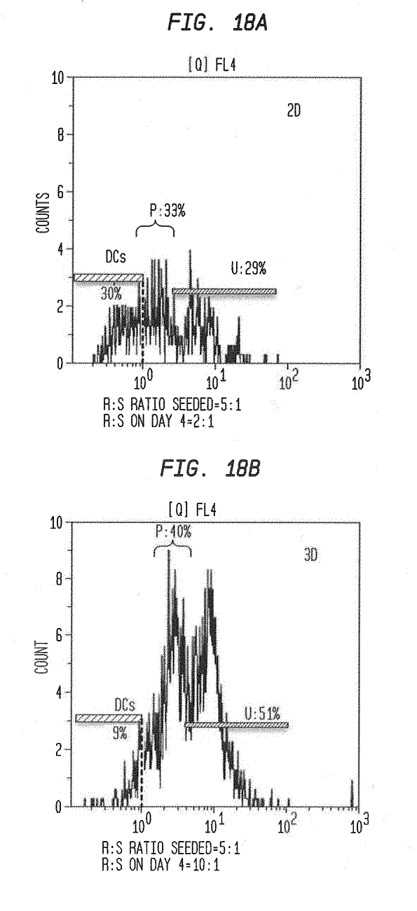

For the m-APC culture chamber, PS microbeads of 90 μm were assembled with m-DCs (from BALB.B mice) to form a 250 μm-thick assembly on the PC membrane surface. Microbeads were pre-conditioned with 100 ng/mL lipopolysaccharides (LPS) to promote the adhesion of m-DCs to the microbead surface (Abdi, K., Singh, N. J. & Matzinger, P. Lipopolysaccharide-activated dendritic cells: “exhausted” or alert and waiting? J. Immunol. 188, 5981-5989, doi: 10.4049/jimmunol.1 102868 (2012)). m-T cells (from eGFP transgenic B6 mice) were labeled with eFIuro® 670 and introduced 24 h later from the top of the microbeads assembly at a R:S of 5:1. Since the packed 90 μm microbeads provide interstitial openings of −14 μm, T cells were able to infiltrate through the microbeads assembly and interact with m-DCs, which were attached to the microbead surface. The m-T cells were circulated for 4 days. As shown in FIGS. 18A and 18B, analyses of live cells (as determined by light forward and side scatters) the P.:S ratio of m-T cells to m-DCs: (1) increased from 5:1 to −10:1 (i.e., m-T cells=90%, m-DCs=9%) in the 3D culture chamber and (2) decreased to −2:1 (i.e., m-T cells=60%, m-DCs=30%) in 2D plate culture. Likewise, while T cells underwent one round of proliferation in both culture conditions (for explanation of analyses, see Zhang et al. Patient-Specific 3D Microfluidic Tissue Model for Multiple Myeloma. Tissue engineering. Part C, Methods 20: 663-670, doi: 10.1089/ten.TEC.2013.0490 (2014)), the percentage of proliferating cells (labeled as “P” in FIGS. 18A and 18B) was greater in 3D than in 2D (40 vs. 33%).

As hypothesized, these results suggest that the circulatory 3D perfusion culture is an effective approach in enhancing the viability, proliferation, and activation of T cells in comparison to conventional 2D co-culture. These enhancements are attributed to the synergistic use of both microbeads and circulatory perfusion in providing m-T cells with significantly higher chances of interacting with m-DCs.

Taken together, these preliminary results strongly support that the device of the described invention can be used for: (1) biomaterials-guided cultures of CRIECs and DCs and (2) T cell circulation through these chambers to facilitate and enhance the viability, proliferation, and activation of reactive T cell population.

### Example 9.8 Experiments to Further Optimize the Use of the Device in Replicating the Stimulation, Circulation and Proliferation Events that Donor T Cells Encounter in the Patient Body and Predicting the Pathologic Potential of Donor T Cells Against Host Epithelium

Experiments to establish that 80% of unstimulated T cells can be circulated through the CRIEC and DC culture chambers for up to 1 week. The effects of biomaterials, flow conditions, and tissue cell presence on the re-circulation of unstimulated T cells from transgenic eGFP B6 mice, in the range of 105 to 106 cells will be quantified. These baseline experiments will be primarily conducted with murine cells, but main results from the experiments will be confirmed using human cells. When tissue cells are not present in the device, it is anticipated that culture medium flow rate, pore size of nanofibrous mesh, and microbead size will have major influences as to how T cells can travel through the culture chambers. (1) The flow rate will be varied in the range of 10 to 50 uL/min; (2) the mesh pore size will be varied from 5 to 10 inn by controlling electrospinning process parameters, and (3) the PS microbead size will be varied, i.e., 45, 75, and 90 urn as these sizes are commercially available (Polyscience). In addition to qualitative visual and microscopic observations at various locations of the device, the percentage changes of circulating T cells (vs. cells that get entrapped in the device) will be quantified by sampling 50-100 uL of the effluent each day for a 1 week period and counting the cells in the collected medium using an automated cell counter. Also, flow cytometry will be performed to follow changes in cell viability on a daily basis for the 1-week period. For the sampling purpose and medium replenishment, a sampling port will be added in the external circulation loop.

After the empty device characterization, how the presence of CRIECs and APCs (i.e., DCs) in these chambers will interfere with the movement of T cells will be studied. For the epithelial culture chamber, experiments after CRIECs reach confluence will be performed, which initial observations indicate takes about 1-4 days.

In preliminary experiments, no evidence of m-CRIECs blocking T cell movements was seen, although such observations to date are limited. The flow rate and biomaterial parameters will be optimized to ensure that >80% can freely be recirculated through the chambers for up to 1 week.

Experiments to Establish that T Cells Become Activated and Persist for 3 Weeks Due to Biomimetic Recirculation.

Due to the recirculatory attribute of iGVHD, Gl miHA-specific T cells continuously stimulated in the APC and tissue chambers are expected to persist and expand over the 3-week benchmark period to cause measurable CRIEC damage. The operation of the APC chamber that can be initially seeded with 105 DCs (sufficient for the stimulation of 106 T cells) will be optimized. m-DCs will be prepared by injecting host mice with B16-FLt3L tumor, which promotes the enrichment of DCs in tumor bearing mice. (Anandasabapathy, N. et al. Classical Flt3L-dependent dendritic cells control immunity to protein vaccine. J. Exptl Med. 211, 1875-1891, doi:10.1084/jem.20131397 (2014); Anandasabapathy, N. et al. Flt3L controls the development of radiosensitive dendritic cells in the meninges and choroid plexus of the steady-state mouse brain. J. Exptl Med. 208, 1695-1705, doi: 10.1084/jem.20102657 (2011)).

Human DCs (h-DCs) will be derived from patient PBL monocytes (Santodonato, L. et al. Monocyte-derived dendritic cells generated after a short-term culture with IFN-alpha and granulocyte-macrophage colony-stimulating factor stimulate a potent Epstein-Barr virus-specific CD8+ T cell response. J. Immunol. 170, 5195-5202 (2003)).

The following experiments will be performed with murine cells first and later confirmed with human cells. As preliminary results suggest, m-DCs can infiltrate into the microbeads assembly and become adhered to the microbead surface. Upon the introduction of T cells and their physical contact, T cells will be activated. Since DCs are programmed to die after maturation (typically within 5 days) and therefore in order to provide constant stimulations (Abdi, K., Singh, N. J. & Matzinger, P. Lipopolysaccharide-activated dendritic cells: “exhausted” or alert and waiting? J. Immunol. 188, 5981-5989, doi: 10.4049/jimmunol.1 102868 (2012)) the capability to replenish dead DCs with new DCs will need to be developed. Simply adding new DCs at 5-day intervals by opening the PDMS plug and placing them onto the top of the microbeads assembly is planned. It is expected that dead cell debris will be washed away and the microbead surface will become available again for the arrival and adhesion of new DCs, since T cells do not adhere to the microbead surface (as observed in preliminary experiments). The effectiveness of the replenishment approach at providing constant T cell stimulation will be evaluated by measuring cell viability, activation, and proliferation at various replenish time intervals (3, 7, 14, 21 days) over 3 weeks. After the APC chamber is optimized, the synergistic effects of CRIECs on T cell viability, activation, and proliferation will be investigated and compared with DCs only. Annexin V/PI staining will be used to determine the viability of T cells. T cell activation will be determined by percent changes in CD25 and CD69 expressions. For the proliferation assay (Zhang, W., Lee, W. Y., Siegel, D. S., Tolias, P. & Zilberberg, J. Patient-Specific 3D Microfluidic Tissue Model for Multiple Myeloma. Tissue Engineering. Part C, Methods 20, 663-670, doi: 10.1089/ten.TEC.2013.0490 (2014)), T cells will be labeled with cell trace carboxyfluorescein succinimidyl ester (CFSE) proliferation dye and analyzed using flow cytometry.

If DCs cannot be replenished by the infiltration approach, replacing the whole assembly and place a new microbead/dendtric cell assembly with T cells separated from the old assembly and re-introduced will be considered. Also, cytokines (e.g., IL-2) can be added to the culture chamber in order to prolonged T cell maintenance (Hedfors, I. A. & Brinchmann, J. E. Long-term proliferation and survival of in vitro-activated T cells is dependent on Interleukin-2 receptor signalling but not on the high-affinity IL-2R. Scandinavian journal of immunology 58, 522-532 (2003)). Moreover, retinoic acid could be added to generate gut-tropic DCs, which should facilitate the generation of T cells with superior 1EC-killing avidity (Gorfu, G., Rivera-Nieves, J. & Ley, K. Role of beta7 integrins in intestinal lymphocyte homing and retention. Current Molec. Med. 9, 836-850 (2009); Agace, W. W. T-cell recruitment to the intestinal mucosa. Trends in Immunol. 29, 514-522, doi: 10.1016/j.it.2008.08.003 (2008)).

The relatively large hole-to-hole distance in the current membrane material may limit the T cell movement through the membranes. Although this was not seen in preliminary experiments with 106 circulating T cells, this may be an issue when the number of circulating T cells is significantly increased to achieve high E:T ratios. If this becomes a problem, using polyethylene terephthalate (PET) membrane (Greiner Bio-One) with the average pore size of 8 μm and the surface pore density of 1.5×106 cm−2 (vs. 105 cm−2 for the current PC membrane) will be considered.

Experiments to Establish that iGVHD can facilitate CRIECs killing within 2 or 3 weeks: After operative procedures are optimized from the above tasks, iGVHD will be used to determine PIdx values using cells from murine GVHD models (Table 6). T cell recirculation is expected to: (1) lower the E:T cell ratio (i.e., the seeding ratio of CRIECs and T cells in iGVHD) to achieve measurable m-CR1EC killing and (2) speed up the killing for the reasons articulated earlier. For these experiments, the E:T ratios will be titrated in the range of 2:1 to 20:1. Because of the plate reader assay capability of the platform, measurement of % cell death for calculation of PIdx is expected to be streamlined using in situ determination of cell death by caspase-3 staining (Luft, T. et al. Serum cytokeratin-18 fragments as quantitative markers of epithelial apoptosis in liver and intestinal graft-versus-host disease. Blood 110, 4535-4542, doi: 10.1182/blood-2006-10-049817 (2007); Disbrow, G. L. et al. Dihydroartemisinin is cytotoxic to papillomavirus-expressing epithelial cells in vitro and in vivo. Cancer Res. 65, 10854-10861, doi:10.1158/0008-5472.CAN-05-1216 (2005)). After iGVHD's facilitated killing capability is established with murine cells, the results will be confirmed using patient-derived cells. Based on these results, overall iGVHD design features and operational protocols will be reviewed and revised as necessary.

Experiments to Correlate Statistically GVHD Risk Predictions from iGVHD from 24 Patient-Donor Samples with Patient Outcomes:

For each patient-donor pair, PIdx will be determined using the iGVHD device and the protocols developed in the previous tasks. The recirculation and high-throughput capabilities of the device will be utilized to evaluate 3 or more E:T ratios. PIdx values determined from 24 patient-donor samples will be compared to patient outcomes as follows. The main outcome of interest, severity of GVHD (grades 0-IV), will be dichotomized in low severity (LS: 0-1) and high severity (HS: 11, III, IV). Discriminant validity of PIdx will be examined by comparing PIdx from LS and HS groups using a two-sided Student's t-test or Wilcoxon rank sum test, as appropriate. Logistic regression analysis will be performed to assess the capability of PIdx as a risk predictor of GVHD. The results of this analysis will be presented as odds ratios (OR), 95% confidence interval, P-value. Area under receiver operating characteristics (ROC) curve will be used to quantify probability of accurate classification of LS vs. HS outcomes. ROC analysis and optimal cut point function based on Youden Index will be used to determine the cutoff value for PIdx. Hochberg procedure will be utilized to adjust for multiple testing. Using the determined cutoff value, sensitivity, specificity, positive predictive value (PPV) and negative predictive value (NPV), overall accuracy will be calculated and reported using standard 2 by 2 tables for categorical analysis.

Follow-On Clinical Study. A follow-on clinical study with prospective sample collection is anticipated after proof of principle of predicting GVHD using human samples is established. Samples of 3 to 4 donors for haploidentical cases can be screened, although corroboration of the predictive outcome will be only for the selected haploidentical donor. Nonetheless, these cases will be use as a proof-of-principle that responses between multiple donors using this approach can be discerned.

### Example 9.9 Circulation of Primary Murine T Cells Through Primary Murine Intestine Epithelial Cells Maintained on Nanofibrous Mesh

Adoptive T cell therapy in the form of allogeneic blood and marrow transplantation (allo-BMT) has proven to be one of the few curative treatments for patients suffering from a number of drug-resistant hematological malignancies. However, the full exploitation of this clinical intervention is greatly limited by graft versus host disease (GVHD), as one of the major BMT complications. This disease is characterized by severe and potentially lethal tissue damage to skin, liver, and gut tissues of transplanted patients, mediated by donor T cells responding to host alloantigens.35-37 In particular, GVHD of the gastrointestinal tissues is closely associated with non-relapse mortality following allo-BMT (A. C. Harris, J. E. Levine and J. L. Ferrara, Clin. Haematol., 2012, 25, 473-478). Currently, there is no way to predict which patient-donor pairs will develop GVHD after BMT. Our long-term interest is to explore the possibility of emulating the potential killing of patient-derived intestinal epithelial cells (IECs) by donor T cells, where IECs are the primary population targeted in GI GVHD (A. M. Hanash, J. A. Dudakov, G. Hua, M. H. O'Connor, L. F. Young, N. V. Singer, M. L. West, R. R. Jenq, A. M. Holland, L. W. Kappel, A. Ghosh, J. J. Tsai, U. K. Rao, N. L. Yim, O. M. Smith, E. Velardi, E. B. Hawryluk, G. F. Murphy, C. Liu, L. A. Fouser, R. Kolesnick, B. R. Blazar and M. R. M. van den Brink, Immunity, 2013, 37, 339-350; R. El-Asady, R. Yuan, K. Liu, D. Wang, R. E. Gress, P. J. Lucas, C. B. Drachenberg and G. A. Hadley, J. Exp. Med., 2005, 201, 1647-1657). In native tissues, IECs reside on a thin fibrous basement membrane (BMa) consisting of the intermingled networks of laminins and collagens and provides cell anchoring and barrier functions. The membrane networks interact with cells through membranous integrin receptors and other plasma membrane molecules, influencing cell differentiation, migration, adhesion, phenotype, and survival.

As an initial step towards this application, we used our prototype device to: (1) culture and maintain primary conditionally reprogrammed murine IECs isolated from the small intestine of a C57Bl/6-TgIJCAG-OVA)916 Jen/J mouse (B6-SIINFEKL) and (2) assess the device's capability in supporting the circulation of primary murine T cells through the IECs (FIG. 9A for conceptual illustration of the experimental approach).

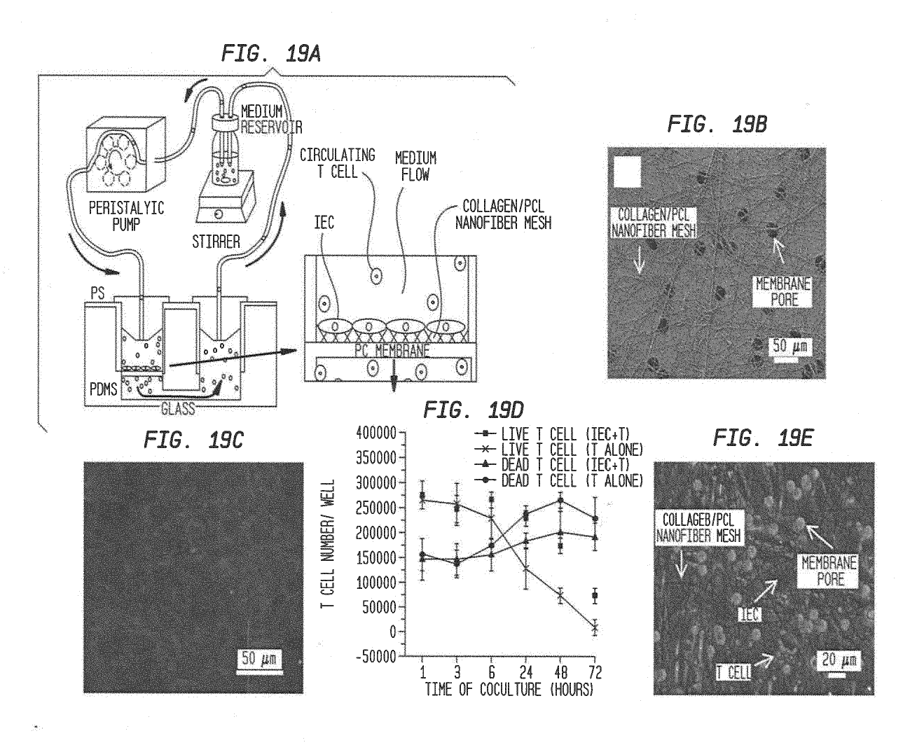

As shown by the scanning electron microscopic (SEM) image in FIG. 19B, nanofiber mesh was used to mimic the BMa of the epithelial tissue as well as to support the long-term viability of IECs. The latter role is particularly important, since primary IECs cannot be kept viable during conventional culture. The nanofiber mesh was produced by electronspinning polycaprolactone (PCL)/type I collagen onto the PC membrane surface prior to the device assembly (X. Yang, K. R. Ogbolu and H. Wang, J. Exp. Nanosci., 2008, 3, 329-345). The average open space in the nanofiber mesh was controlled to about 6 μm (FIG. 19B) since the average diameter of T cells is about 5 μm. For the same reason, we also selected the average pore of the PC membrane to be 10 μm (FIG. 19B). With the use of nanofiber mesh, IECs were able to develop into a confluent layer and exhibit cobblestone morphology while remaining viable in the perfusion device for up to 7 days (FIG. 19C).

After IECs became confluent (approximately 4 days post seeding), enriched T cells obtained from an eGFP transgenic C57Bl6/J mouse were introduced and circulated through the culture chambers (2.5×105 cells per chamber). As illustrated in FIG. 19A, a peristaltic pump was used to circulate T cells in RPMI complete medium. Visually, we did not see the entrapment of T cells in any part of the culture device and external circulatory pathways. T cell viability was quantified by sampling the culture medium at various time points and counting live and dead T cell numbers suspended in the medium. As shown in FIG. 19D, the overall viability of T cells decreased during the 72 h culture period. This was expected since it is well known that the viability of T cells cannot be maintained in vitro unless they are stimulated by antigen-presenting cells or maintained via the addition of cytokines like IL-2 (P. Marrack and J. Kappler, Annu. Rev. Immunol., 2004, 22, 765-787). However, interestingly, there were more viable T cells when they were circulated through the IEC layer. The results suggest that T cells were activated by IECs, resulting in the increased viability of T cells. Although both B6-SIINFEKL IECs and T cells were of B6 background, it is likely that minor antigen differences between the B6-SIINFEKL and the eGFP-B6 strains could have elicited activation of T cells and potentially other cells like natural killer (NK) cells. FIG. 19E shows that IECs were spreading on the nanofiber mesh surface, and were in physical contact with T cells. Since the membrane pores (10 μm) and opening spaces (>6 μm) between nanofibers were larger than the T cells (5 μm), they were able to go through the IEC layer without getting trapped in the culture chamber.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

1. An ex vivo model of a three dimensional (3D) cellular network found in native bones via biomimetic assembly of osteocytes and microbeads in a microfluidic perfusion culture device comprising
(a) preparing an in vitro multiwell plate-based perfusion culture device, comprising, from top to bottom:
1. a bottomless multi-well plate comprising a plurality of bottomless wells;
2. a first micropatterned polymer layer attached to a bottom surface of the bottomless multi-well plate to form a plurality of adjacent wells, one or more of each pair of adjacent wells comprising a transparent polymer membrane placed within the one or more of each pair of adjacent wells;
3. a second micropatterned polymer layer comprising two or more holes that correspond to two or more adjacent wells, the second micropatterned polymer layer being attached to a bottom surface of the first micropatterned polymer layer, such that each hole of the second micropatterned polymer layer is aligned with the two or more adjacent wells in the first micropatterned polymer layer, one or more of each pair of adjacent wells comprising the transparent polymer membrane;
4. a microfluidic channel formed between the two adjacent wells that allows internal fluidic communication between the two adjacent wells;
5. one or more removable polymer plugs, each located at a top surface of each of the plurality of wells, and one or more tubes, each connected to the one or more polymer plugs;
6. a pump connected to a reservoir that removably connects to the tubes;
7. a transparent, optical grade glass layer attached to the bottom surface of the second micropatterned polymer layer that forms a bottom surface for the plurality of wells and that seals the multi-well plate perfusion culture device;
wherein
(i) one or more of the two adjacent wells is a cell culture chamber comprising a first well region including a first well and a second well region including a second well;
(ii) the microfluidic channel connects the first well region and the second well region with one another;
(iii) the first well is adapted to receive a therapeutic agent, the second well is adapted to receive a biological sample of cells; and
(iv) liquids, nutrients and dissolved gas molecules flow through the channel
(b) constructing an ex vivo endosteal microenvironment perfused by nutrients and dissolved gas molecules by;
1. seeding a surface of the culture chamber of the device of (a) with
(i) microbeads;
(ii) osteocyte cells (OSTs); and
(iii) osteoblast cells (OSBs), and
2. culturing the cells with a culture medium through the microfluidic channel for a time effective for the cells to form three-dimensional (3D) nodular structures that comprise a 3D-endosteal-like tissue.

2. A method for selecting a patient-specific treatment for multiple myeloma (MM) comprising:
(a) preparing the ex vivo endosteal microenvironment perfused by nutrients and dissolved gas molecules comprising three-dimensional (3D) nodular structures that comprise a 3D-endosteal-like tissue according to claim 1;
(b) acquiring bone marrow mononuclear cells (BMMCs) comprising viable multiple myeloma cells (MMCs) from a subject;
(c) bringing the BMMCs comprising viable MMCs in contact with the endosteal microenvironment perfused by nutrients and gas molecules to seed the ex vivo endosteal microenvironment with the viable MMCs, the ex vivo endosteal microenvironment perfused by nutrients and gas molecules seeded with viable MMCs forming an ex vivo microenvironment effective to recapitulate spatial and temporal characteristics of a multiple myeloma cancer niche and to maintain viability of the MMCs from the subject; and
(d) testing therapeutic efficacy of a therapeutic agent on the viable MMCs maintained by the endosteal microenvironment in the first well adapted to receive a therapeutic agent by
1. contacting the MMCs maintained by the endosteal microenvironment of (d) with a test therapeutic agent; and
2. comparing at least one of viability and level of apoptosis of the MMCs contacted with the test therapeutic agent to an untreated MMC control, and
(e) initiating therapy to treat the subject with the test therapeutic agent if the test therapeutic agent is effective to significantly reduce viability of the MMCs contacted with the test therapeutic agent or to increase apoptosis of the MMCs contacted with the test therapeutic agent compared to the untreated MMC control.

3. The method according to claim 2, wherein the microbeads are biphasic calcium phosphate (BCP) microbeads, polystyrene (PS) microbeads or a combination thereof.

4. The method according to claim 2, wherein the microbeads range in diameter from about 20 μm to about 25 μm.

5. The method according to claim 2, wherein the osteocyte cells are primary human osteocytes (ph-OSTs) or murine osteocytes.

6. The method according to claim 2, wherein the osteoblast cells (OSBs) are primary human osteoblasts (ph-OSBs).

7. The method according to claim 5, wherein the primary human osteoblasts (ph-OSBs) are autologous ph-OSBs.

8. The method according to claim 2, wherein the gas molecules are oxygen (O₂) molecules.

9. The method according to claim 2, wherein the therapeutic agent is selected from the group consisting of a chemotherapeutic agent, a corticosteroid, an immunomodulating agent, a proteasome inhibitor, a histone deacetylase (HDAC) inhibitor, a monoclonal antibody and interferon.

10. The method according to claim 9, wherein the chemotherapeutic agent is selected from the group consisting of melphalan, vincristine, cyclophosphamide, etoposide, doxorubicin, liposomal doxorubicin and bendamustine.

11. (canceled)

12. (canceled)

13. (canceled)

14. (canceled)

15. (canceled)

16. (canceled)

17. An ex vivo method for assessing drug resistance of multiple myeloma cells (MMCs) in a subject suffering from multiple myeloma (MM) comprising:
(a) preparing the ex vivo endosteal microenvironment perfused by nutrients and dissolved gas molecules comprising three-dimensional (3D) nodular structures that comprise a 3D-endosteal-like tissue according to claim 1;
(b) acquiring bone marrow mononuclear cells (BMMCs) comprising viable multiple myeloma cells (MMCs) from the subject;
(c) bringing the BMMCs comprising viable MMCs in contact with the endosteal microenvironment perfused by nutrients and gas molecules to seed the ex vivo endosteal microenvironment with the viable MMCs, the ex vivo endosteal microenvironment perfused by nutrients and gas molecules seeded with viable MMCs forming an ex vivo microenvironment effective to recapitulate spatial and temporal characteristics of a multiple myeloma cancer niche and to maintain viability of the MMCs from the subject; and
(d) testing therapeutic efficacy of a therapeutic agent on the viable MMCs maintained by the endosteal microenvironment in the first well adapted to receive a therapeutic agent by
1. contacting the MMCs maintained by the endosteal microenvironment of (d) with a test therapeutic agent; and
2. comparing at least one of viability and level of apoptosis of the MMCs contacted with the test therapeutic agent to an untreated MMC control,
wherein the MMCs are resistant to the test therapeutic agent if the test therapeutic agent is not effective to significantly reduce viability of the MMCs or is not effective to increase apoptosis of the MMCs compared to the untreated MMC control.

18. The method according to claim 17, wherein the microbeads are biphasic calcium phosphate (BCP) microbeads, polystyrene (PS) microbeads or a combination thereof.

19. The method according to claim 17, wherein the microbeads range in diameter from about 20 μm to about 25 μm.

20. The method according to claim 17, wherein the osteocyte cells are primary human osteocytes (ph-OSTs) or murine osteocytes.

21. The method according to claim 17, wherein the osteoblast cells (OSBs) are primary human osteoblasts (ph-OSBs).

22. The method according to claim 21, wherein the primary human osteoblasts (ph-OSBs) are autologous ph-OSBs.

23. The method according to claim 17, wherein the gas molecules are oxygen (O₂) molecules.

24. The method according to claim 17, wherein the therapeutic agent is selected from the group consisting of a chemotherapeutic agent, a corticosteroid, an immunomodulating agent, a proteasome inhibitor, a histone deacetylase (HDAC) inhibitor, a monoclonal antibody and interferon.

25. The method according to claim 17, wherein the chemotherapeutic agent is selected from the group consisting of melphalan, vincristine, cyclophosphamide, etoposide, doxorubicin, liposomal doxorubicin and bendamustine.

26. (canceled)

27. (canceled)

28. (canceled)

29. (canceled)

30. (canceled)

31. The method according to claim 17, wherein the interferon is selected from the group consisting of interferon-α, interferon-β, interferon-γ and interferon-λ.
